SEQUENCE LISTING (55 sequences)

SEQ ID NO: 1 - DNA, 20 bp, Artificial Sequence
  Description of Artificial Sequence Synthetic oligonucleotide
tgcagagacatagtttctga

SEQ ID NO: 2 - DNA, 20 bp, Artificial Sequence
  Description of Artificial Sequence Synthetic oligonucleotide
agaaactatgtctctgcaga

SEQ ID NO: 3 - DNA, 20 bp, Artificial Sequence
  Description of Artificial Sequence Synthetic oligonucleotide
aacatagccttaattcagcc

SEQ ID NO: 4 - DNA, 20 bp, Artificial Sequence
  Description of Artificial Sequence Synthetic oligonucleotide
tgagaagcctggctgaatta

SEQ ID NO: 5 - DNA, 1584 bp, Artificial Sequence
  Description of Artificial Sequence Synthetic polynucleotide
atgtcactccaaatggtgaccgtgagcaacaatattgctctcatccaaccgggtttctctctcatgaacttcgacggccaggtgtttttcttcgggcagaagggctggccaaaacggtcatgcccgactggtgtatttcatttggacgtgaagcacaatcatgtgaagctcaagccgactatcttcagcaaggacagctgttacctgcccccgcttaggtatcctgcaacatgtacatttaagggctccctggaaagcgagaagcaccagtatattatccacggcggcaagacccctaataacgaagttagcgacaagatatacgtgatgagtatagtgtgcaaaaacaataagaaggtcacgtttcgatgcaccgagaaggacctcgtgggagacgtgccggaggctcgctacggccatagcatcaatgttgtatatagtagaggaaagtctatgggcgtacttttcggcggccgatcttatatgccctcaacacatcgcacgactgagaagtggaactcagtggcagattgcctcccttgcgtgttcctggtcgacttcgagttcggttgcgctacctcatatatacttcccgagctgcaggacggactgtctttccatgtgtcaatcgcgaagaacgataccatctacatcctcggtggtcacagcctcgcgaacaacattcgacctgcaaatctgtacaggattagagtagacctcccgcttgggagtcccgcggtcaattgtactgtccttccggggggcatatccgtcagctccgccatcctgacccagaccaataacgacgagttcgtcattgtcggtggttatcaacttgagaatcagaagaggatgatatgtaacatcatcagccttgaagacaacaaaatagagattagagagatggagacgcccgactggacacccgacatcaaacatagcaaaatctggttcggctcaaacatgggcaacgggacggtgttcctgggcatccctggagataacaagcaggtggtgtccgaggggttttacttctacatgttgaagtgcgcggaggacgacaccaacgaggaacagactacatttaccaactcccaaacaagtaccgaggaccccggcgattctacacccttcgaggattcagaagagttttgcttcagcgctgaagctaactctttcgacggcgatgacgagttcgatacatacaacgaggacgacgaggaggacgaaagcgaaactggatactggatcacctgctgccccacgtgcgacgtggacataaacacctgggtgccgttctactcaaccgaactgaataagccagcaatgatctactgttcacatggtgatggacactgggtgcacgcccagtgcatggacctcgctgagagaactctcattcacttgagtgcaggatctaacaaatattattgcaacgagcacgtggagatcgcccgcgcacttcacacaccgcagcgagtgttgcccttgaagaagccccctatgaagagccttaggaagaagggcagcggcaagatactgacgcccgcgaaaaagagcttcctgaggagactgttcgactaa

SEQ ID NO: 6 - DNA, 1584 bp, Artificial Sequence
  Description of Artificial Sequence Synthetic polynucleotide
atgagcctgcagatggtgaccgtgtccaacaatatcgccctgatccagccaggcttttctctgatgaatttcgacggccaggtgttctttttcggccagaagggatggccaaagcggagctgcccaaccggcgtgtttcacctggacgtgaagcacaaccacgtgaagctgaagcctacaatcttctctaaggatagctgctacctgccccctctgagataccccgccacctgtacattcaagggctccctggagtctgagaagcaccagtacatcatccacggcggcaagacacctaacaatgaggtgtctgataaaatctacgtgatgagcatcgtgtgcaagaacaataagaaggtgacctttcggtgtacagagaaggacctggtgggcgatgtgccagaggcaagatacggccacagcatcaatgtggtgtattccagaggcaagtctatgggcgtgctgttcggcggcaggagctacatgccctccacccaccgcaccacagagaagtggaactccgtggccgactgcctgccttgcgtgttcctggtggattttgagttcggctgtgccacctcttatatcctgcctgagctgcaggacggcctgtctttccacgtgagcatcgccaagaatgatacaatctacatcctgggcggccactccctggccaacaatatcaggccagccaatctgtatcggatcagagtggacctgcctctgggcagcccagcagtgaactgcaccgtgctgccaggaggcatcagcgtgagcagcgccatcctgacccagacaaacaatgacgagtttgtgatcgtgggcggctaccagctggagaaccagaagaggatgatctgtaatatcatcagcctggaggataacaagatcgagatccgcgagatggagacccccgactggacacctgacatcaagcacagcaagatttggtttggctccaacatgggcaatggcacagtgttcctgggcatccccggcgataacaagcaggtggtgtccgagggcttttacttctatatgctgaagtgcgccgaggacgataccaatgaggagcagaccaccttcaccaacagccagacctccacagaggaccctggcgattctaccccattcgaggacagcgaggagttttgtttctccgccgaggccaattcttttgacggcgacgatgagttcgatacctacaacgaggacgatgaggaggatgagagcgagacaggctattggatcacctgctgtcctacatgcgacgtggacatcaatacctgggtgccattctactccacagagctgaacaagcccgccatgatctattgctctcacggcgacggacactgggtgcacgcacagtgtatggatctggccgagaggaccctgatccacctgtctgccggcagcaacaagtactattgtaatgagcacgtggagatcgcccgggccctgcacacaccacagagagtgctgcctctgaagaagccacccatgaagtccctgcgcaagaagggctctggcaagatcctgacccccgccaagaagtcctttctgcggagactgttcgactga

SEQ ID NO: 7 - DNA, 3247 bp, Artificial Sequence
  Description of Artificial Sequence Synthetic polynucleotide
tcctttgctcccgtctagtcacttcgcaccttcggctagtctttattcacattccaggagtcccctctcccagagcaacaaagacagatttccaaagctacacatcccagagagaacggattcttgggaaatgtggttctttcagctgacgcatggtgactctttacggaaaaggactacaattcccagaaatcctagggagcatatagtccgtggctaaaacatgtcccagctccttggatggaatggcagtaaaggttctgtggctctttactgacctaactccttggattttcctcattcagtcccactgcaagcgtgtgggaggacttaaaaaaatgctattcacatgtgaaggaatctaaatacgatgattatatgaagcgatctctaagtcattttattttataattctttcagacaaaaatctacgtaggatcagaaactatgagcctgcagatggtgaccgtgtccaacaatatcgccctgatccagccaggcttttctctgatgaatttcgacggccaggtgttctttttcggccagaagggatggccaaagcggagctgcccaaccggcgtgtttcacctggacgtgaagcacaaccacgtgaagctgaagcctacaatcttctctaaggatagctgctacctgccccctctgagataccccgccacctgtacattcaagggctccctggagtctgagaagcaccagtacatcatccacggcggcaagacacctaacaatgaggtgtctgataaaatctacgtgatgagcatcgtgtgcaagaacaataagaaggtgacctttcggtgtacagagaaggacctggtgggcgatgtgccagaggcaagatacggccacagcatcaatgtggtgtattccagaggcaagtctatgggcgtgctgttcggcggcaggagctacatgccctccacccaccgcaccacagagaagtggaactccgtggccgactgcctgccttgcgtgttcctggtggattttgagttcggctgtgccacctcttatatcctgcctgagctgcaggacggcctgtctttccacgtgagcatcgccaagaatgatacaatctacatcctgggcggccactccctggccaacaatatcaggccagccaatctgtatcggatcagagtggacctgcctctgggcagcccagcagtgaactgcaccgtgctgccaggaggcatcagcgtgagcagcgccatcctgacccagacaaacaatgacgagtttgtgatcgtgggcggctaccagctggagaaccagaagaggatgatctgtaatatcatcagcctggaggataacaagatcgagatccgcgagatggagacccccgactggacacctgacatcaagcacagcaagatttggtttggctccaacatgggcaatggcacagtgttcctgggcatccccggcgataacaagcaggtggtgtccgagggcttttacttctatatgctgaagtgcgccgaggacgataccaatgaggagcagaccaccttcaccaacagccagacctccacagaggaccctggcgattctaccccattcgaggacagcgaggagttttgtttctccgccgaggccaattcttttgacggcgacgatgagttcgatacctacaacgaggacgatgaggaggatgagagcgagacaggctattggatcacctgctgtcctacatgcgacgtggacatcaatacctgggtgccattctactccacagagctgaacaagcccgccatgatctattgctctcacggcgacggacactgggtgcacgcacagtgtatggatctggccgagaggaccctgatccacctgtctgccggcagcaacaagtactattgtaatgagcacgtggagatcgcccgggccctgcacacaccacagagagtgctgcctctgaagaagccacccatgaagtccctgcgcaagaagggctctggcaagatcctgacccccgccaagaagtcctttctgcggagactgttcgactgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctatgtctctgcagatggtaacagtcagtaataacatagccttaattcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttc

SEQ ID NO: 8 - DNA, 84 bp, Artificial Sequence
  Description of Artificial Sequence Synthetic oligonucleotide
ccatcagaaactatgtctctgcagatggtaacagtcagtaataacatagccttaattcagccaggcttctcactgatgaatttt

SEQ ID NO: 9 - DNA, 84 bp, Artificial Sequence
  Description of Artificial Sequence Synthetic oligonucleotide
aaaattcatcagtgagaagcctggctgaattaaggctatgttattactgactgttaccatctgcagagacatagtttctgatgg

SEQ ID NO: 10 - DNA, 75 bp, Artificial Sequence
  Description of Artificial Sequence Synthetic oligonucleotide
  modified_base (26)..(26): a, c, t, g, unknown or other
tcagacaaaaatctacgtaccatcangaaactatgtctctgcagatggtaacagtcagtaataacatagccttaa

SEQ ID NO: 11 - DNA, 65 bp, Artificial Sequence
  Description of Artificial Sequence Synthetic oligonucleotide
tcagacaaaaatctacgtaccatgtctctgcagatggtaacagtcagtaataacatagccttaat

SEQ ID NO: 12 - DNA, 75 bp, Artificial Sequence
  Description of Artificial Sequence Synthetic oligonucleotide
tcagacaaaaatctacgtaccatcagaaactatgtctctgcagatggtaacagtcagtaataacatagccttaat

SEQ ID NO: 13 - DNA, 65 bp, Artificial Sequence
  Description of Artificial Sequence Synthetic oligonucleotide
tcagacaaaaatctacgtaccatctctctgcagatggtaacagtcagtaataacatagccttaat

SEQ ID NO: 14 - DNA, 58 bp, Artificial Sequence
  Description of Artificial Sequence Synthetic oligonucleotide
tcagacaaaaatctacgtaccatcagatggtaacagtcagtaataacatagccttaat

SEQ ID NO: 15 - DNA, 69 bp, Artificial Sequence
  Description of Artificial Sequence Synthetic oligonucleotide
tcagacaaaaatctacgtaccatctatgtctctgcagatggtaacagtcagtaataacatagccttaat

SEQ ID NO: 16 - DNA, 61 bp, Artificial Sequence
  Description of Artificial Sequence Synthetic oligonucleotide
tcagacaaaaatctacgtaccatctgcagatggtaacagtcagtaataacatagccttaat

SEQ ID NO: 17 - DNA, 74 bp, Artificial Sequence
  Description of Artificial Sequence Synthetic oligonucleotide
tcagacaaaaatctacgtaccatcaaaactatgtctctgcagatggtaacagtcagtaataacatagccttaat

SEQ ID NO: 18 - DNA, 65 bp, Artificial Sequence
  Description of Artificial Sequence Synthetic oligonucleotide
tcagacaaaaatctacgtactatgtctctgcagatggtaacagtcagtaataacatagccttaat

SEQ ID NO: 19 - DNA, 73 bp, Artificial Sequence
  Description of Artificial Sequence Synthetic oligonucleotide
tcagacaaaaatctacgtaccatgaaactatgtctctgcagatggtaacagtcagtaataacatagccttaat

SEQ ID NO: 20 - DNA, 74 bp, Artificial Sequence
  Description of Artificial Sequence Synthetic oligonucleotide
tcagacaaaaatctacgtaccatcgaaactatgtctctgcagatggtaacagtcagtaataacatagccttaat

SEQ ID NO: 21 - DNA, 67 bp, Artificial Sequence
  Description of Artificial Sequence Synthetic oligonucleotide
tcagacaaaaatctacgtaccatctgtctctgcagatggtaacagtcagtaataacatagccttaat

SEQ ID NO: 22 - DNA, 70 bp, Artificial Sequence
  Description of Artificial Sequence Synthetic oligonucleotide
tcagacaaaaatctacgtaccatcctatgtctctgcagatggtaacagtcagtaataacatagccttaat

SEQ ID NO: 23 - DNA, 72 bp, Artificial Sequence
  Description of Artificial Sequence Synthetic oligonucleotide
tcagacaaaaatctacgtaccatcaactatgtctctgcagatggtaacagtcagtaataacatagccttaat

SEQ ID NO: 24 - DNA, 26 bp, Artificial Sequence
  Description of Artificial Sequence Synthetic probe
cccgaggaacgtgaccatggagtggc

SEQ ID NO: 25 - DNA, 20 bp, Artificial Sequence
  Description of Artificial Sequence Synthetic primer
gcacaggaagtttagcagtg

SEQ ID NO: 26 - DNA, 20 bp, Artificial Sequence
  Description of Artificial Sequence Synthetic primer
gggaattcaagacgctcaga

SEQ ID NO: 27 - DNA, 26 bp, Artificial Sequence
  Description of Artificial Sequence Synthetic probe
gagcctgcagatggtgaccgtgtcca

SEQ ID NO: 28 - DNA, 20 bp, Artificial Sequence
  Description of Artificial Sequence Synthetic primer
gcaccttcggctagtcttta

SEQ ID NO: 29 - DNA, 20 bp, Artificial Sequence
  Description of Artificial Sequence Synthetic primer
atcagagaaaagcctggctg

SEQ ID NO: 30 - DNA, 21 bp, Artificial Sequence
  Description of Artificial Sequence Synthetic primer
atgtggttctttcagctgacg

SEQ ID NO: 31 - DNA, 22 bp, Artificial Sequence
  Description of Artificial Sequence Synthetic primer
cgaaaagtaacctttttgttgt

SEQ ID NO: 32 - DNA, 23 bp, Artificial Sequence
  Description of Artificial Sequence Synthetic oligonucleotide
tgcagagacatagtttctgatgg

SEQ ID NO: 33 - DNA, 23 bp, Artificial Sequence
  Description of Artificial Sequence Synthetic oligonucleotide
tgcatagtcattgtttctgatgg

SEQ ID NO: 34 - DNA, 23 bp, Artificial Sequence
  Description of Artificial Sequence Synthetic oligonucleotide
taaagagacatattttctgaggg

SEQ ID NO: 35 - DNA, 18 bp, Artificial Sequence
  Description of Artificial Sequence Synthetic primer
atgtggttctttcagctg

SEQ ID NO: 36 - DNA, 20 bp, Artificial Sequence
  Description of Artificial Sequence Synthetic primer
tctcacctcccattccctag

SEQ ID NO: 37 - DNA, 20 bp, Artificial Sequence
  Description of Artificial Sequence Synthetic primer
tcagggcgatattgttggac

SEQ ID NO: 38 - DNA, 18 bp, Artificial Sequence
  Description of Artificial Sequence Synthetic probe
tcacttcgcaccttcggc

SEQ ID NO: 39 - DNA, 17 bp, Artificial Sequence
  Description of Artificial Sequence Synthetic probe
cgtgaccatggagtggc

SEQ ID NO: 40 - DNA, 20 bp, Artificial Sequence
  Description of Artificial Sequence Synthetic primer
tcacctgttcaaaagtcccc

SEQ ID NO: 41 - DNA, 18 bp, Artificial Sequence
  Description of Artificial Sequence Synthetic primer
tggttgtgcttcacgtcc

SEQ ID NO: 42 - DNA, 26 bp, Artificial Sequence
  Description of Artificial Sequence Synthetic primer
agatggtgtcatttttggcaatagag

SEQ ID NO: 43 - DNA, 25 bp, Artificial Sequence
  Description of Artificial Sequence Synthetic oligonucleotide
tggagctgagsagsctgagatcyga

SEQ ID NO: 44 - DNA, 19 bp, Artificial Sequence
  Description of Artificial Sequence Synthetic oligonucleotide
aacccacasagaccctcac

SEQ ID NO: 45 - DNA, 23 bp, Artificial Sequence
  Description of Artificial Sequence Synthetic oligonucleotide
tccctkaractctcctgtrcagc

SEQ ID NO: 46 - DNA, 22 bp, Artificial Sequence
  Description of Artificial Sequence Synthetic oligonucleotide
ctacaacccstccctcaagagt

SEQ ID NO: 47 - DNA, 25 bp, Artificial Sequence
  Description of Artificial Sequence Synthetic oligonucleotide
cagcaccgcctacctgcagtggagc

SEQ ID NO: 48 - DNA, 18 bp, Artificial Sequence
  Description of Artificial Sequence Synthetic oligonucleotide
tccggggacagtgtctct

SEQ ID NO: 49 - DNA, 24 bp, Artificial Sequence
  Description of Artificial Sequence Synthetic oligonucleotide
cagcacrgcatayctgcagatcag

SEQ ID NO: 50 - DNA, 22 bp, Artificial Sequence
  Description of Artificial Sequence Synthetic oligonucleotide
aagtagtccttgaccaggcagc

SEQ ID NO: 51 - DNA, 19 bp, Artificial Sequence
  Description of Artificial Sequence Synthetic oligonucleotide
ggagacgagggggaaaagg

SEQ ID NO: 52 - protein, 20 aa, Artificial Sequence
  Description of Artificial Sequence Synthetic peptide
MSLQMVTVSNNIALIQPGFS

SEQ ID NO: 53 - DNA, 20 bp, Artificial Sequence
  Description of Artificial Sequence Synthetic oligonucleotide
agtctttgatacagagacgt

SEQ ID NO: 54 - DNA, 74 bp, Artificial Sequence
  Description of Artificial Sequence Synthetic oligonucleotide
accatcagaaactatgtctctgcagatggtaacagtcagtaataacatagccttaattcagccaggcttctcac

SEQ ID NO: 55 - DNA, 74 bp, Artificial Sequence
  Description of Artificial Sequence Synthetic oligonucleotide
gtgagaagcctggctgaattaaggctatgttattactgactgttaccatctgcagagacatagtttctgatggt

(19) United States
(12) Patent Application Publication
(10) Pub. No.: US 2024/0173355 A1
(43) Pub. Date: May 30, 2024

(54) GENE CORRECTION FOR RAG2 DEFICIENCY IN HUMAN STEM CELLS

(71) Applicant: The Board of Trustees of the Leland Stanford Junior University, Stanford, CA (US)

(72) Inventors: Mara Pavel-Dinu, Stanford, CA (US); Matthew H. Porteus, Stanford, CA (US)

(73) Assignee: The Board of Trustees of the Leland Stanford Junior University, Stanford, CA (US)

(21) Appl. No.: 18/551,975

(22) PCT Filed: Apr. 5, 2022
(86) PCT No.: PCT/US2022/071549

§ 371 Date: Sep. 22, 2023

Related U.S. Application Data
(60) Provisional application No. 63/170,935, filed on Apr. 5, 2021.

(51) Int. Cl. A61K 35/28; A61K 35/545; A61P 37/04; C12N 5/074; C12N 5/0789; C12N 9/22; C12N 15/11; C12N 15/86

(52) U.S. Cl. CPC A61K 35/28; A61K 35/545; A61P 37/04; C12N 5/0647; C12N 5/0696; C12N 9/22; C12N 15/111; C12N 15/86; C12N 2310/20; C12N 2750/14143

(57) ABSTRACT

The present disclosure provides methods and compositions for treating RAG2 deficiencies in subjects, comprising genetically modifying cells from the subjects ex vivo by integrating a functional, codon-optimized RAG 2 cDNA at the endogenous RAG2 locus.

### CROSS REFERENCE TO RELATED APPLICATIONS

The present application claims priority to U.S. Provisional Pat. Appl. No. 63/170,935, filed on Apr. 5, 2021, which application is incorporated herein by reference in its entirety.

### STATEMENT AS TO RIGHTS TO INVENTIONS MADE UNDER FEDERALLY SPONSORED RESEARCH AND DEVELOPMENT

This invention was made with Government support under contract 5U54AI082973-13 awarded by the National Institutes of Health. The Government has certain rights in the invention.

### BACKGROUND

Severe combined immune deficiency (SCID) comprises an array of inherited genetic defects that affect the development of T lymphocytes (and, in some cases, also B and/or NK cells), thereby compromising adaptive immune responses. Patients with SCID are highly susceptible to serious infections from birth and inevitably die within the first years of life unless treated with allogeneic hematopoietic stem cell transplantation (HSCT). The Recombination Activating Genes 1 and 2 (RAG1 and RAG2) proteins initiate the process of V(D)J recombination that gives rise to a diverse repertoire of T and B cell receptors (TCRs, BCRs), thereby allowing recognition of antigens and adaptive immune responses [1]. Mutations in RAG1 or RAG2 can result in various clinical phenotypes [2]. Functionally null mutations cause a complete arrest of T and B cell development, resulting in T⁻ B⁻ NK⁺ SCID, whereas hypomorphic variants that allow residual RAG function are partially permissive to T (and in some cases, B) cell development, and often manifest with immune dysregulation as a result of faulty negative selection of self-reactive cells, in addition to infections [3].

Currently, the only definitive cure for RAG deficiency is represented by allogeneic HSCT; however, this treatment comes with an array of possible complications, including graft vs. host disease and transplant-related toxicities. Furthermore, challenges exist in finding matched donors for select ethnic groups, and graft failure and incomplete immune reconstitution have been frequently reported, especially after unconditioned haploidentical HSCT [4]. Previous preclinical attempts to correct RAG deficiency by gammaretrovirus- or lentivirus-mediated gene transfer in mice have led to controversial results, reflecting inadequate and/or dysregulated expression of the RAG genes using heterologous promoters [5-11].

Targeted gene insertion or gene editing using site-specific nucleases, including CRISPR (clustered regularly interspaced short palindromic repeats)/Cas9 (CRISPR-associated 9) nuclease, zinc-finger nucleases (ZFNs), or transcription activator-like effector nucleases (TALENs), has potential for gene therapy with a greatly reduced or absent risk of insertional mutagenesis. In these approaches, a DNA double-strand break (DSB) induced by the nuclease acts as a target for homology-directed repair (HDR) using a donor DNA template containing homologous sequences to those flanking the cut site. Due to the toxicity of double-stranded plasmid DNAs in transfections of primary cells, adeno-associated virus (AAV) vectors packaged with serotype 6 capsid (AAV6) are commonly used for transduction of HSPCs to deliver long donor DNA templates (with an upper size limit of ˜4.7-kb for AAV vector packaging, including target site homologous sequences and ˜0.3-kb for the required AAV inverted terminal repeats or ITRs), while single-stranded oligodeoxynucleotides (ssODNs) have been used for gene repair or insertion of short donor templates (typically up to 100-200 nucleotides in length including homologous sequences). The efficiency of targeted insertion of a donor DNA template is dependent upon the choice of DSB repair pathways between HDR and non-homologous end joining (NHEJ), a more error-prone repair pathway that functions without a homologous donor template and can instead result in the formation of indels (insertion or deletion mutations) at the DSB site. This choice of repair pathway appears to be cell type and cell cycle dependent, with HDR normally restricted to S and G2 phases of the cell cycle, which poses an additional challenge for HDR-mediated genome editing in quiescent hematopoietic stem cells.

There is a need for new, safe, and effective methods for correcting RAG2 deficiencies in patients that avoid complications such as GVH disease and that provide levels of RAG2 expression sufficient to enable the development of T and B cells. The present disclosure satisfies this need and provides other advantages as well.

### BRIEF SUMMARY

The present disclosure provides methods and compositions for treating RAG2 deficiencies in subjects, in particular through the genetic modification of cells taken from the subjects by integrating a functional, codon-optimized RAG2 cDNA at the endogenous RAG2 locus, and subsequently reintroducing the modified cells back into the subject. In particular, the present methods and compositions involve the homologous-recombination-mediated introduction of functional, codon-optimized RAG2 cDNAs into the genomes of cells at the RAG2 locus, such that the functional RAG2 cDNA is expressed in the cells under the control of the endogenous RAG2 promoter and other regulatory elements.

In one aspect, the present disclosure provides a method of genetically modifying a cell from a subject with a Recombination-Activating Gene 2 (RAG2) deficiency, the method comprising: introducing into a cell isolated from the subject a single guide RNA (sgRNA) targeting the RAG2 gene, an RNA-guided nuclease, and a homologous donor template comprising a RAG2 cDNA comprising a nucleotide sequence having at least 80% identity to SEQ ID NO:5 or SEQ ID NO:6, flanked by a first and a second RAG2 homology region; wherein the sgRNA binds to the nuclease and directs it to a target sequence within the RAG2 gene, whereupon the nuclease cleaves the gene at the target sequence, and wherein the cDNA is integrated by homology directed recombination (HDR) at the site of the cleaved RAG2 gene, such that the cDNA is expressed under the control of the endogenous RAG2 promoter, thereby providing functional RAG2 protein product in the cell.

In some embodiments, the method further comprises isolating the cell from the subject prior to the introducing of the sgRNA, RNA-guided nuclease, and homologous donor template. In some embodiments, the target sequence of the sgRNA is within exon 3 of the RAG2 gene, and wherein the RAG2 cDNA comprises exon 3 of the RAG2 gene. In some embodiments, the sgRNA comprises a nucleotide sequence complementary to a sequence selected from the group consisting of SEQ ID NO:1, SEQ ID NO:2, SEQ ID NO:3, or SEQ ID NO:4. In some embodiments, the sgRNA comprises a nucleotide sequence complementary to SEQ ID NO: 1. In some embodiments, the sgRNA comprises 2′-O-methyl-3′-phosphorothioate (MS) modifications at one or more nucleotides. In some embodiments, the 2′-O-methyl-3′-phosphorothioate (MS) modifications are present at the three terminal nucleotides of the 5′ and 3′ ends.

In some embodiments, the RNA-guided nuclease is Cas9. In some embodiments, the Cas9 is a high fidelity Streptococcus pyogenes Cas9 (SpCas9). In some embodiments, the sgRNA and the RNA-guided nuclease are introduced into the cell as a ribonucleoprotein (RNP). In some embodiments, the RNP is introduced into the cell by electroporation. In some embodiments, the method further comprises introducing i53, e.g., i53 mRNA or recombinant i53 protein, into the cell. In some embodiments, the i53 mRNA or protein is introduced by electroporation together with the RNP.

In some embodiments, the RAG2 cDNA comprises a nucleotide sequence having at least 85%, 90%, 95%, 96%, 97%, 98%, 99%, 99.5%, 99.7%, 99.9% or more identity to SEQ ID NO:5 or SEQ ID NO:6. In some embodiments, the RAG2 cDNA comprises the nucleotide sequence of SEQ ID NO:5 or SEQ ID NO:6. In some embodiments, the homologous donor template further comprises a polyadenylation signal at the 3′ end of the cDNA, where both the cDNA and the polyadenylation signal are flanked by the first and the second RAG2 homology regions on the template. In some such embodiments, the polyadenylation signal is a bovine growth hormone polyadenylation signal. In some embodiments, the first and/or second RAG2 homology region comprises nucleotides 1-447 or 2848-3247 of SEQ ID NO:7, or a contiguous portion of nucleotides 1-447 or 2848-3247 of SEQ ID NO:7. In some embodiments, the first and second RAG2 homology regions comprise nucleotides 1-447 or 2848-3247 of SEQ ID NO:7. In some embodiments, the homologous template further comprises a woodchuck hepatitis virus post-transcriptional regulatory element (WPRE). In some embodiments, the homologous donor template is introduced into the cells using a recombinant adeno-associated virus (rAAV) serotype 6 vector. In some embodiments, the homologous donor template further comprises a selectable marker.

In some embodiments, the cell is an induced pluripotent stem cell (iPSC). In some embodiments, the iPSC is derived from a fibroblast isolated from the subject. In some embodiments, the cell is a hematopoietic stem and progenitor cell (HSPC). In some embodiments, the modified cell is heterozygous for the integrated RAG2 cDNA. In some embodiments, the modified cell is homozygous for the integrated RAG2 cDNA. In some embodiments, the modified cell can differentiate into a human embryonic mesodermal progenitor (hEMP) cell in vitro. In some embodiments, the modified cell can differentiate into a T cell in vitro. In some embodiments, the T cell is selected from the group consisting of CD34⁺ CD7⁺ CD5⁻; CD7⁺, CD5⁺, CD1a⁺; CD4⁺ CD8⁺; and CD3⁺ TCRαβ+; CD3⁺ TCRγδ⁺.

In another aspect, the present disclosure provides a method of treating a subject with a RAG2 deficiency, comprising (i) genetically modifying a cell from the subject using any of the herein-described methods, and (ii) reintroducing the cell into the subject.

In some embodiments of the method, the cell is reintroduced into the subject by systemic transplantation. In some embodiments, the systemic transplantation comprises intravenous administration. In some embodiments, the cell is reintroduced into the subject by local transplantation. In some embodiments, the local transplantation comprises interfemoral or intrahepatic administration. In some embodiments, the cell is cultured and/or selected prior to being reintroduced into the subject.

In some embodiments, the sgRNA comprises a nucleotide sequence complementary to the sequence of any one of SEQ ID NOS: 1-4. In some embodiments, the sgRNA comprises 2′-O-methyl-3′-phosphorothioate (MS) modifications at one or more nucleotides. In some embodiments, the 2′-O-methyl-3′-phosphorothioate (MS) modifications are present at the three terminal nucleotides of the S′ and 3′ ends.

In another aspect, the present disclosure provides a homologous donor template comprising: (i) a RAG2 cDNA comprising a nucleotide sequence comprising at least 80% identity to SEQ ID NO:5 or SEQ ID NO:6; (ii) a first RAG2 homology region located to one side of the cDNA within the donor template; and (iii) a second RAG2 homology region located to the other side of the cDNA within the donor template.

In some embodiments of the donor template, the first RAG homology region comprises nucleotides 1-447 of SEQ ID NO:7, or a contiguous portion thereof, and the second RAG2 homology region comprises nucleotides 2848-3247 of SEQ ID NO:7, or a contiguous portion thereof. In some embodiments, the RAG2 cDNA comprises exon 3 of the RAG2 gene. In some embodiments, the RAG2 cDNA comprises a nucleotide sequence having at least 85%, 90%, 95%, 96%, 97%, 98%, 99% or more identity to SEQ ID NO:5 or 6. In some embodiments, the RAG2 cDNA is codon optimized. In some embodiments, the RAG2 cDNA comprises the nucleotide sequence of SEQ ID NO:5 or 6. In some embodiments, the donor template further comprises a polyadenylation signal at the 3′ end of the RAG2 cDNA, wherein both the cDNA and the polyadenylation signal are flanked by the first and second RAG2 homology regions on the template. In some embodiments, the polyadenylation signal is a bovine growth hormone polyadenylation signal. In some embodiments, the template comprises the sequence of SEQ ID NO: 7. In some embodiments, the donor template further comprises a selectable marker. In some embodiments, the donor template further comprises a woodchuck hepatitis virus post-transcriptional regulatory element (WPRE).

In another aspect, the present disclosure provides an isolated iPSC or HSPC comprising any of the herein-described sgRNAs and/or homologous donor templates.

In another aspect, the present disclosure provides an isolated, genetically modified iPSC or HSPC comprising an exogenous, codon-optimized RAG2 cDNA integrated at the endogenous RAG2 locus, wherein the integrated cDNA comprises a nucleotide sequence having at least 80% identity to SEQ ID NO:5 or 6.

In some embodiments, the RAG2 cDNA comprises a nucleotide sequence having at least 85%, 90%, 95%, 96%, 97%, 98%, 99%, 99.5%, 99.7%, 99.9% or more identity to SEQ ID NO:5 or 6. In some embodiments, the RAG2 cDNA comprises the nucleotide sequence of SEQ ID NO:5 or 6. In some embodiments, the exogenous RAG2 cDNA comprises exon 3 of the RAG2 gene, and wherein the cDNA is integrated within exon 3 of the endogenous RAG2 gene. In some embodiments, the iPSC or HSPC was modified using any of the herein-described methods.

In another aspect, the present disclosure provides a pharmaceutical composition comprising a plurality of genetically modified iPSCs and/or HSPCs comprising an exogenous, codon-optimized RAG2 cDNA integrated at the endogenous RAG2 locus, wherein the integrated cDNA comprises a nucleotide sequence having at least 80% identity to SEQ ID NO:5 or 6.

In some embodiments, the composition further comprises non-genetically modified iPSCs and/or HSPCs or iPSCs and/or HSPCs comprising INDELs at the RAG2 locus. In some embodiments, the composition is comprised of at least 5% of genetically modified iPSCs and/or HSPCs comprising the integrated RAG2 cDNA. In some embodiments, the composition is comprised of 9% to 50% of genetically modified iPSCs and/or HSPCs comprising the integrated RAG2 cDNA.

### BRIEF DESCRIPTION OF THE DRAWINGS

FIGS. 1A-1G. Characterization of iPSCs and gene editing. FIG. 1A: q-PCR based quantification of mRNA expression levels of pluripotency markers in fibroblast derived iPSCs benchmarked against fibroblast mRNA. FIG. 1B. Karyotype results of the RAG2 deficient patient cell line reprogrammed to pluripotency. FIG. 1C. Illustration of the gene editing strategy, showing the AAV cargo being integrated into the RAG2 locus. FIG. 1D. Representative data showing on-target sgRNA activity as measured by INDEL frequencies at the RAG2 locus. FIG. 1E, ddPCR data showing enrichment for the r.RAG2 integrated cargo in a heterogeneous pool of RAG2 deficient patient iPSCs. FIG. 1F. DNA electrophoresis gel showing three clones with integration of the r.RAG2 cassette across the 3″ junction of the cargo and the chromosomal DNA. FIG. 1G. G-banding of the clone edited with the r.RAG2 cassette on both alleles showing normal karyotype.

FIGS. 2A-2D: Human iPSC derived T cell differentiation in healthy donor cells and in RAG2 deficient and RAG2 edited cells. FIG. 2A: Representative analysis of the flow cytometry gating showing expression of early and mature T cell differentiation markers for a healthy donor sample. FIG. 2B. Flow cytometry results of T cell differentiation markers for the healthy donor cells, the RAG2 deficient patient cells, and the RAG2 gene-edited cells showing co-expression of CD5 and CD7, along with CD4, CD8a, and in the case of the healthy donor and edited cells, also CD3 and TCRαβ. FIG. 2C. Absolute count of indicated iPSC-derived T cell subsets per ATO in the healthy donor sample, the RAG2 deficient sample, and the gene-edited sample. FIG. 2D. Representative data showing differentiation profiles for the three examined samples over three distinct experiments. Statistical significance was computed using multiple paired t-tests with a Holm-Sidak correction.

FIGS. 3A-3C: Analysis of VJ recombination and TCR repertoire in healthy and edited iPSC derived T cells. FIG. 3A. Heatmaps showing the pairings of V and J genes for both the TRA/TRD and TRB loci in healthy donor and RAG2 deficient gene-edited CD3-sorted cells. V and J genes are listed in 5′ to 3′ order according to their physical location on the chromosome. FIG. 3B. Treemap showing diversity of TCRα CDR3 sequences in sorted CD3⁺ cells differentiated in vitro from healthy donor and RAG2-deficient gene-edited patient-derived iPSCs. Each dot represents a unique CDR3 sequence and the size of each dot is scaled to the frequency of that CDR3 being present in the total number of reads. FIG. 3C. Shannon H Index score for TCRα. CDR3 sequences in sorted CD3⁺ cells from indicated samples, showing generation of a diverse repertoire of CD3⁺ cells from healthy donor and patient-derived, RAG2 gene-edited iPSCs. A Shannon index value of 8 typically denotes polyclonality.

FIG. 4: Location and sequence identity of screened RAG2 sgRNAs. Depiction of sgRNA locations screened to target the start codon of RAG2 for full cDNA insertion using CRISPR/AAV6. Small boxes proximal to each sgRNA and of the same color represent the protospacer adjacent motif (PAM) requisite for SpCas9 activity.

FIG. 5: Quantification of HDR events in iPSCs by ddPCR. Analysis of initial genomic integration of the cargo delivered by AAV6 upstream of exon 3 of the RAG2 locus by ddPCR. Transduced cells were split into two wells before sib-selection.

FIG. 6: Differentiation of human embryonic mesodermal progenitors from iPSCs. FACS data showing derivation of the human embryonic mesodermal progenitor (hEMP) intermediate from RAG2 deficient patient iPSCs, edited iPSCs. and healthy donor iPSCs as notated by expression of CD56 and lack of expression of EPCAM.

FIG. 7: Representative flow cytometry data showing supplemental markers of T cell development including CD1a and TCRγδ as well as markers of NK cell (CD56), monocyte (CD14), HSC (CD34), and B cell (CD19) development for each of the healthy donor, RAG2 deficient patient, and edited cell lines.

FIG. 8: Virtual Spectratyping of iPSC-derived mature T cells. Virtual CDR3 Spectratyping shows similar CDR3 lengths in both TCRAD and TCRB chains from respective heterogeneous pools of healthy donor and edited patient T cells expressing CD3.

FIGS. 9A-9B: Analysis of VJ recombination and TCR repertoire in healthy and edited iPSC-derived T cells. FIG. 9A: Heatmaps showing the pairings of V and J genes for both the TRA/TRD and TRB loci in healthy donor and RAG2-deficient gene-edited CD3⁺ sorted cells. V and J genes are listed in 5′ to 3′ order according to their physical location on the chromosome. FIG. 9B: Treemap showing diversity of TCRα CDR3 sequences in sorted CD3⁺ cells differentiated in vitro from healthy donor and RAG2-deficient gene-edited patient-derived iPSCs. Each dot represents a unique CDR3 sequence and the size of each dot is scaled to the frequency of that CDR3 being present in the total number of reads.

FIGS. 10A-10B: RAG2 Corrective Donors. FIG. 10A: Map for corrective donor version 1 (sequence shown as SEQ ID NO:5). FIG. 10B: Map for corrective donor version 2 (sequence shown as SEQ ID NO:6).

FIGS. 11A-11C: Overview of genome targeting of SCID-RAG2 HSPCs. RAG2 Patient (c.296>A: c.1342C>A) HSPCs' Pre- and Post-Gene Targeting. FIG. 11A: Ex vivo proliferation of compound heterozygous SCID-RAG2 HSPCs Days 0-3 pre-genome targeting. FIG. 11B: Days 3-5 post genome targeting. FIG. 11C: Overview of genome targeting outcome of SCID-RAG2 patient derived HSPCs treated i53 and 2.500 MOI AAV6 delivering coOpt version 2 RAG2 cDNA. HR=homologous recombination, INDELs=insertion/deletions, Un-edited (wild type); 153=inhibitor of 53BP1.

FIGS. 12A-12B: Overview of SCID-RAG2 HSPCs engraftment. FIG. 12A: Peripheral blood analysis at week 8. FIG. 12B: Bone marrow aspirate analysis at week 12 post-engraftment. 500,000 mutant HSPCs (un-corrected HSPCs) black filled circles; 500,000 coOpt RAG2 cDNA corrected grey triangle and 1,000,000 coOpt RAG2 cDNA corrected red squares.

FIG. 13: FACS plots showing bone marrow engraftment of SCID-RAG2 HSPCs un-corrected (Mut) and corrected with 1 million corrected HSPCs. Each plot represents one unique mouse.

FIG. 14: FACS plots showing bone marrow engraftment of SCID-RAG2 HSPCs un-corrected (Mut) and corrected with 500,000 corrected HSPCs. Each plot represents one unique mouse.

FIG. 15: FACS plots showing bone quantification of CD19″ B cells. In the marrow of NSG-SGM3 mice engrafted with SCID-RAG2 HSPCs un-corrected (Mut) and corrected with 1 million corrected HSPCs. Show are FACS plots from mice showing phenotypic correction.

FIG. 16: FACS plots showing bone quantification of CD19⁺ IgM⁺ B cells. In the marrow of NSG-SGM3 mice engrafted with SCID-RAG2 HSPCs un-corrected (Mut) and corrected with 1 million and 500,000 corrected HSPCs. Show are FACS plots from mice showing phenotypic correction.

FIG. 17: FACS plots showing bone quantification of CD3⁺ T cells. In the marrow of NSG-SGM3 mice engrafted with SCID-RAG2 HSPCs un-corrected (Mut) and corrected with 1 million corrected HSPCs. Show are FACS plots from mice showing phenotypic correction.

FIG. 18: FACS plots showing bone quantification of CD4⁺ T cells (mature T-cells). In the marrow of NSG-SGM3 mice engrafted with SCID-RAG2 HSPCs un-corrected (Mut) and corrected with 500,000 corrected HSPCs. Shown are FACS plots from mice showing phenotypic correction.

FIGS. 19A-19B: Engraftment summary. FIG. 19A: Week 20 post-engraftment FACS-based analysis of bone marrow derived from NSG-SGM3 mice engrafted with 250,000 umbilical cord blood HSPCs. or (FIG. 19B) frozen mobilized peripheral blood HSPCs. Conditions: wild type (white), mock-targeted (grey), coOpt RAG2 v2 cDNA gene targeted in the absence (blue) or presence (orange) of i53 inhibitor.

FIGS. 20A-20B. Engraftment summary. FIG. 20A: Week 20 post-engraftment FACS based analysis of spleen derived from NSG-SGM3 mice engrafted with 250,000 umbilical cord blood HSPCs. or (FIG. 20B) frozen mobilized peripheral blood HSPCs. Conditions: wild type (white), mock-targeted (grey), coOpt RAG2 v2 cDNA gene targeted in the absence (blue) or presence (orange) of i53 inhibitor.

FIGS. 21A-21B: Overview of multi-lineage analyses of healthy donor derived HSPCs RAG2 gene targeted and engrafted. FIG. 21A. Week 20 post engraftment of fresh umbilical cord blood HSPCs, or (FIG. 21B) frozen mobilized peripheral blood HSPCs. Control=wild type and mock-targeted, RNP=ribonucleoprotein only targeted; GT=gene targeted.

FIGS. 22A-22C: Post-engraftment analysis of coOpt RAG2 lymphoid cells. FIG. 22A: Sorted human T (CD3⁺) from bone marrow (BM), or (FIG. 22B) sorted human B (CD19⁺ and CD20⁺ IgM⁺) cells from spleen (SP) at week 20 post transplant using fresh cord blood (CB), or (FIG. 22C) frozen peripheral blood (PB). Quantification of coOpt RAG2 cDNA in sorted cells by ddPCR analysis. Bars: mean±s.e.m. Each symbol represents an individual mouse.

FIG. 23: Schematics of the hematopoietic developmental defect in RAG2-SCID patients. Overview of the human T-cell (top panel) and B-cell (bottom panel) developmental stages in thymus and bone marrow from common lymphoid progenitor (CLP). Dotted red squares mark the developmental block in RAG2-SCID patients. RAG2, recombination activating gene 2; DN, double negative stage; ISP, immature single positive stage; EDP, early double-positive stage; DP, double-positive stage.

FIGS. 24A-24D: Efficacy and safety of genome targeting the RAG2 locus using “universal” correction strategy. FIG. 24A. Schematics of gene-targeted integration of codon-optimized RAG2 cDNA (coRAG2) and expression cassette, coRAG2 sequence is under the control of the endogenous promoter. FIG. 24B. Percent coRAG2 gene-targeted (coRAG2-GT) in healthy donor (HD)-purified hematopoietic stem cells (HSPCs) from fresh cord blood (CB) and frozen peripheral blood (PB). Each circle represents a unique human HSPC donor. Genome targeted integration was quantified by digital-droplet PCR (ddPCR). FIG. 24C. Frequency of cells with one (mono-allelic) or two (bi-allelic) alleles targeted, as a function of virus' MOI. Analysis was done on single cells sorted onto methylcellulose plates (n=5-5.000 MOI; n=4-2.500 MOI; n=2-1.250 MOI). Bars±s.e.m. FIG. 24D. Off-target analysis using RAG2-SCID (c.296C>A: c.1342C>A) patient-derived HSPCs. Next-generation sequencing (NSG) of 48 COSMID predicted off-target (OT) sites in edited-only (RNP-sgRNA guide #3 and HiFi Cas9 nuclease) or electroporated-only (mock, nucleofected without RNP). Shown INDELs reads for on target (RAG2 gene, white circle), OT sites below the limit of detection (grey circles), and above the limit of detection (green circles).

FIGS. 25A-25F: Characterization of RAG2 sgRNA-3. FIG. 25A. Schematic of sgRNAs (1-4) binding sites at the RAG2 genomic locus. Orange highlighted sequence indicates the untranslated region and green highlighted sequence marks the RAG2 gene open reading frame, starting from the transcription start site (ATG, marked in bold). FIG. 25B. Assessing genome editing efficiency (INDELs frequency) with the indicated RAG2 sgRNAs. INDELs (insertion and deletions) quantified 48 h post-nucleofection by Sanger sequencing. FIG. 25C. Percent INDELs generated by sgRNA-3 in cord blood (CB) and peripheral blood (PB)-derived HSPCs FIG. 25D. FACS based quantification of CD3⁺ T-cells at week 18, post-Tx into immunodeficient mice, using healthy donor-derived UC HSPCs. A 4-fold decrease in bone marrow (BM) and (FIG. 25E) 3.1-fold decrease in the spleen (SP) in CD3⁺ T cells derived from RNP treated conditions (orange. BM and red triangle, BM) as compared to mock-treated control (white triangles). Each triangle represents an individual mouse. FIG. 25F. INDEL spectrum generated by RAG2 sgRNA-3, 82% alleles acquired INDELS at 48 hours post-RNP-treatment using HiFi Cas9 nuclease. INDEL types and combinations are shown, sgRNA-3 sequence and PAM site is boxed. Analysis was done using Synthego ICE software. Bars: median.

FIGS. 26A-26C: Colony-forming units (CFU) potential of coRAG2 gene targeted HSPCs. FIG. 26A. Colony-forming units (CFU) derived from coRAG2-GT CB and (FIG. 26B) PB-derived HSPCs. Non-gene targeted (non-GT, wild type HPSCs), mock GT (nucleofected only), or GT with coRAG2 cDNA at various AAV6 multiplicity of infection (MOIs). Multi-potential granulocyte, erythroid, macrophage, megakaryocyte progenitor cells (CFU-GEMM, red) granulocytes, macrophage, or both (CFU-G/M/GM, light grey), erythroid progenitors burst-forming unit (BFU-E, yellow), and CFU erythroid (CFU-E, dark grey) n=4 unique CB-derived HPSC donors for WT, mock, and GT using 5,000 MOI, n=2 unique PB-derived HSPCs donors for GT using 2,500 MOI and 1,250 MOI. HSPCs treated (+) or not (−) with p53 inhibitor (153). FIG. 30C. Percent coRAG2-targeted alleles (colored bars) and non-targeted alleles (white bars) for each CFU type. Quantification by ddPCR. 360 total colonies scored.

FIGS. 27A-27B: Distribution of mono- and bi-allelic modification in coRAG2 gene-targeted healthy donor-derived HSPCs, coRAG2 gene-targeted CB-derives HSPCs from donor 1 (FIG. 27A) and (FIG. 27B) donor 2 using three different AAV6 MOIs were single cells FACS sorted into one well of a 96-well plate coated with methylcellulose containing human stem cells specify cytokines. CFU colonies were genotyped 14-days post-sorting. A three primer “in-out” PCR reaction was developed to distinguish and quantify the alleles carrying the coRAG2 cDNA from the wild-type alleles.

FIGS. 28A-28E: In vivo lymphoid lineage development from coRAG2-targeted healthy donor-derived HSPCs. FIG. 28A. Schematic of engraftment protocol of human HSPCs into NSG immunodeficient mice and secondary analysis. FIG. 28B. Percent human cell chimerism (CD45⁺ HLA A-B-C⁺ double-positive cells) in bone marrow (BM) and spleen (SP) of mice 22-weeks post-transplant with CB (intra-hepatic injection) or (FIG. 28C) PB-derived HSPCs (intra-femoral injection) edited with RAG2-sgRNA guide 3 (RNP, light grey circles in FIG. 28B) or targeted with coRAG2 cassette (GT, yellow and red circles in panel B, dark grey and red circles in FIG. 28C). Each dot represents an individual mouse; Panel B: WT (n=10), RNP (n=5). GT (n=11); Panel C: WT (n=5), GT-i53 (n=5), GT+i53 (n=6). Bars=median. FIG. 28D. Percent coRAG2-GT by ddPCR before (−Tx) and after (+Tx) engraftment of CB and (FIG. 28E) PB-derived CD34⁺ HSPCs into NSG mice and FACS sort, as marked. Bars: mean #s.e.m.; −i53, without p53 inhibitor; +i53, with p53 inhibitor; stats. One-Way ANOVA, non-parametric test. Kruskal-Wallis test. Dunn's multiple comparisons test.

FIGS. 29A-29B: Karyotype analysis post-RNP treatment in RAG2″ patient-derived HSPCs. FIG. 29A. RAG2-SCID (c.296C>A; c.1342C>A) HSPCs were nucleofected alone or (FIG. 29B) with sgRNA-3 and HiFi Cas9 nuclease (RNP). G-band karyotype analysis was carried out on 20 cells per condition. No clonal abnormalities were detected at the 375-425 band resolution for the mock-treated and at 350-400 for the RNA-treated samples.

FIGS. 30A-30D: In vivo long-term human hematopoietic reconstitution from coRAG2-targeted healthy donor-derived HSPCs. FIG. 30A. FACS-based quantification of bone marrow (BM) and (FIG. 30B) spleen (SP) primary human engraftment (hCD45⁺ HLA A-B-C⁺) derived from coRAG2-targeted fresh CB HSPCs. Each FACS plot represents an individual mouse. FIG. 30C. Endpoint analysis (22 weeks post-Tx) of human hematopoietic lineage distribution in the BM and peripheral blood of immunocompetent mice (NSG). CD235⁺ (erythroid), CD3⁺ (T-cells), CD19⁺ (B-cells). CD14⁺ (monocytes); n=5 mice (mock), n=11 (coRAG2-GT). FIG. 30D. CD3⁺ T cell subsets (CD4⁺, CD8⁺, CD4⁺CD8⁺) derived in the spleen of NSG mice from (FIG. 30B) engrafted with coRAG2-GT HSPCs.

FIG. 31: Human engraftment and tri-lineage gating scheme. Gating strategy was used for data presented in FIGS. 28A, 28B, 28E, 28F, 32B, 32E, 34A, 34B, 34C, and 30.

FIGS. 32A-32F: Ex vivo gene-targeted RAG2-HSPCs corrects in vivo B-cell developmental block. FIG. 32A. Percent of total genome editing (INDELs and HR) in RAG2^{-/-} patient-derived HSPCs, using four different AAV6 production lots. AAV6 lot D (asterisk marked) was used for all subsequent engraftment studies. FIG. 32B. Percent human cells (CD45⁺ HLA A-B-C⁺) engrafted in BM after transplanting (22 weeks post-Tx) 0.5×10⁶ un-corrected RAG2^{-/-} SCID patient-derived HSPCs (n=4 mice, grey circles), 0.5×10⁶ coRAG2-GT HSPCs (n=15 mice, half red circles) or 1.0×10⁶ coRAG2-GT HSPCs (n=15 mice, black circles). Healthy donor (HD) HSPCs were used as control (n=6, white circles). RAG2^{-/-}-SCID patient genotype: c.296C>A: c.1342C>A. FIG. 32C. FACS based quantification shown in (FIG. 32D) of large Pre-B I, small Pre-B II, immature and mature B cells derived from coRAG2-GT HSPCs. Each population is graphed as a percent of total B cells. Bars: mean±s.e.m. FIG. 32D. Representative FACS plots of B-cell developmental stages derived from a healthy donor (left panel), RAG2^{-/-} patient (middle panel), and coRAG2-GT RAG2^{-/-}. FACS based quantification of percent cells in each developmental stage is shown. FIG. 32E. FACS based quantification of CD19⁺CD20⁺ IgM⁺ triple-positive B cells derived from each condition tested. FIG. 32F. PCR-based sequencing of immunoglobulin M (IgM) heavy chain (Vh) families from sorted triple-positive B-cells.

FIG. 33: B-lymphoid development from coRAG2-GT of RAG^{-/-} SCID patient-derived HSPCs. The gating strategy is used for data presented in FIGS. 34A, 34B, 34C, 34D, and 34E.

FIGS. 34A-34F: Correction of RAG2 gene function in RAG2^{-/-} HSPCs restores V(D)J activity and normal T-cells development. FIG. 34A. Percent human cells (CD45″ HLA A-B-C⁺) detected in spleen (SP) (22-weeks post-Tx) with coRAG2-GT RAG2/HSPC (0.5×10⁶, half-colored red circles or 1.0×10⁶, full-colored red circles). HD (white circles) and un-corrected RAG^{-/-} (grey circles) HSPCs-derived human cells were engrafted and analyzed in parallel. FIG. 34B. Human CD3⁺ T-cells detected in the spleen (SP) and (FIG. 34C) bone marrow (BM) derived from coRAG2-GT RAG2⁺ HSPCs. FIG. 34D. FACS plots showing T-cells analysis derived from 3 mice with the highest level of human CD3⁺ cells (dotted squares). Functional V(D)J rearrangement is demonstrated by the presence of CD3⁺ TCR α/β, CD3⁺ TCR γ/δ, and single-positive CD4⁺ and CD8⁺ derived from coRAG2-GT RAG2-4-HSPCs. FIG. 34E. Treemap diversity analysis for TCRA/TCRD CDR3 sequences from sorted CD3⁺ cells from FIG. 34C. Each circle is a unique CDR3 sequence, and the size of the circle represents the frequency out of the total number of reads. FIG. 34F. Shannon H index score quantification of CDR3 sequence from (FIGS. 34E and 34F), showing oligoclonal repertoire. Shannon index score of ≥8 indicates polyclonal repertoire. Stats. One-way ANOVA, nonparametric, Kruskal-Wallis test. Median plotted in FIGS. 34A, 34B, and 34C.

FIG. 35: Absolute numbers of lymphoid cells generated from RAG2^{-/-} gene-targeted HSPCs and engrafted into immunodeficient mice.

FIGS. 36A-36D: Heatmaps of V and J genes pairing for TCR A/D and TCR beta of T-lymphocytes sorted from gene-targeted (GT) RAG2-SCID patient-derived HSPCs and engrafted into immunodeficient mice. FIG. 36A. Heatmaps of V and J genes pairing for TCR A/D in healthy donors. FIG. 36B, coRAG2-GT or for (FIG. 36C) TCR B in healthy donors and (FIG. 36D) coRAG2-GT and derived CD3⁺ sorted cells. V and J genes are listed in the order of their chromosomal location.

FIG. 37: Virtual spectratyping of RAG2^{-/-} patient and healthy donor-HSPCs derived mature T-cells. Similar CDR3 lengths in TCRAD chains were obtained from the pools of wild type (healthy donor) and genome-targeted patient T cells expressing CD3⁺.

FIGS. 38A-38B: Phenotypic correction of NK-lymphocytes defect in RAG2-SCID patient-derived HSPCs. FIG. 38A. FACS plots showing human NK-cells development in the bone marrow of immunodeficient mice (22 weeks post-Tx). Cells gated on CD3⁻. FIG. 38B. The absolute number of NK cells derived from RAG2-SCID patients (n=4 mice, black diamond), coRAG2-GT patient cells (n=15, half diamonds; n=15, yellow diamonds), and healthy donors (control, n=7, white diamonds). Stats: one-way ANOVA, nonparametric. Dunn's multiple comparison tests. Bars: mean±s.e.m.

### DETAILED DESCRIPTION

### 1. Introduction

The present disclosure provides methods and compositions for the treatment of Recombination-activating Gene 2 (RAG2) Deficiency in subjects, through the introduction and integration at the endogenous RAG2 locus of a functional, codon-optimized RAG2 cDNA. The methods involve the introduction of ribonucleoproteins (RNPs) comprising single guide RNAs (sgRNAs) and RNA-guided nucleases (e.g., Cas9) into cells from the subject, as well as the introduction of homologous templates for repair. The cDNAs are integrated at the endogenous RAG2 gene, e.g., at the translation start site, or within the third exon (or first protein coding exon), such that the cDNA is expressed under the control of the endogenous RAG2 promoter and other regulatory elements and functional RAG2 protein is produced in the cell, thereby compensating for the genetic deficiency in the subject.

In particular embodiments, the RNP complexes. e.g., complexes comprising RAG2-targeting sgRNA and Cas9 protein, are delivered to cells via electroporation, followed by the transduction of the homologous template using an AAV6 viral vector. In some embodiments, i53, e.g., i53 mRNA or protein, is also introduced by electroporation, e.g., at the same time as the RNP. The homologous templates for repair are constructed to have arms of homology centered around the cut site within the RAG2 locus, located on either side of the cDNA on the template. Transcription is terminated using an exogenous polyadenylation signal. This system can be used to modify any human cell, and in particular embodiments induced pluripotent stem cells (iPSCs) or hematopoietic stem and progenitor cells (HSPCs) are used.

### 2. General

Practicing the present methods utilizes routine techniques in the field of molecular biology. Basic texts disclosing the general methods of use in the present methods and compositions include Sambrook and Russell, Molecular Cloning. A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized. e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an.” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid”. “nucleotide”, or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs. SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Obtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a polynucleotide or polypeptide is “heterologous” to an organism if the polynucleotide or polypeptide originates from a foreign species compared to the organism or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of an RAG2 cDNA or encoded protein. In some embodiments, the term refers to the production of a transcriptional and/or translational product encoded by a gene or a portion thereof. The level of expression of a DNA molecule in a cell may be assessed on the basis of either the amount of corresponding mRNA that is present within the cell or the amount of protein encoded by that DNA produced by the cell.

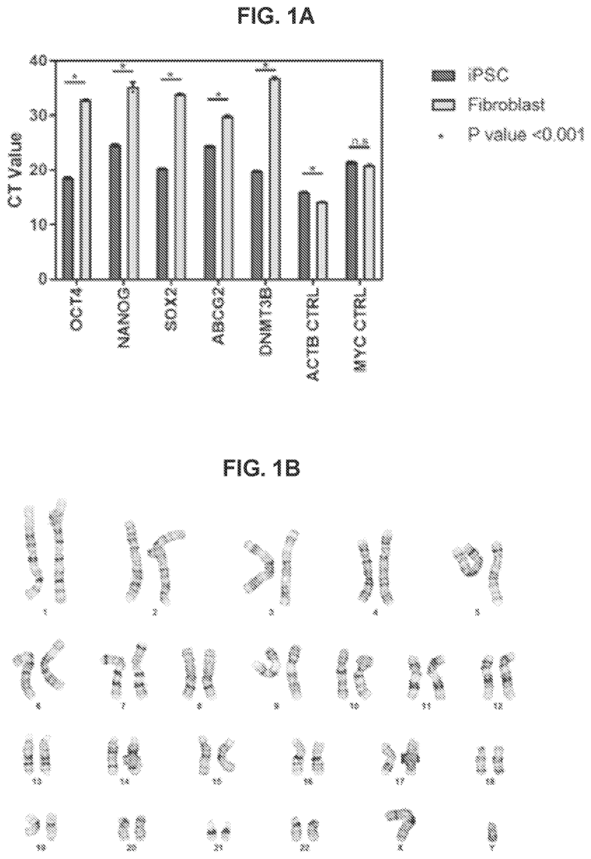

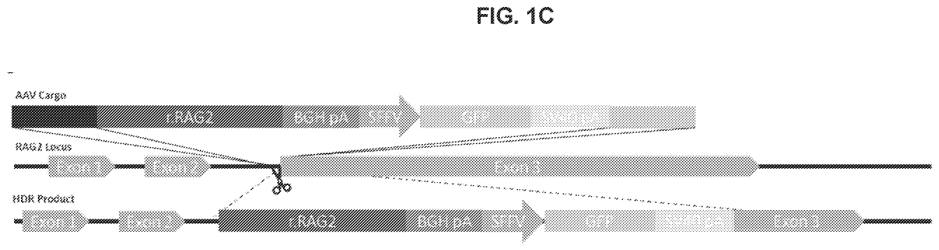

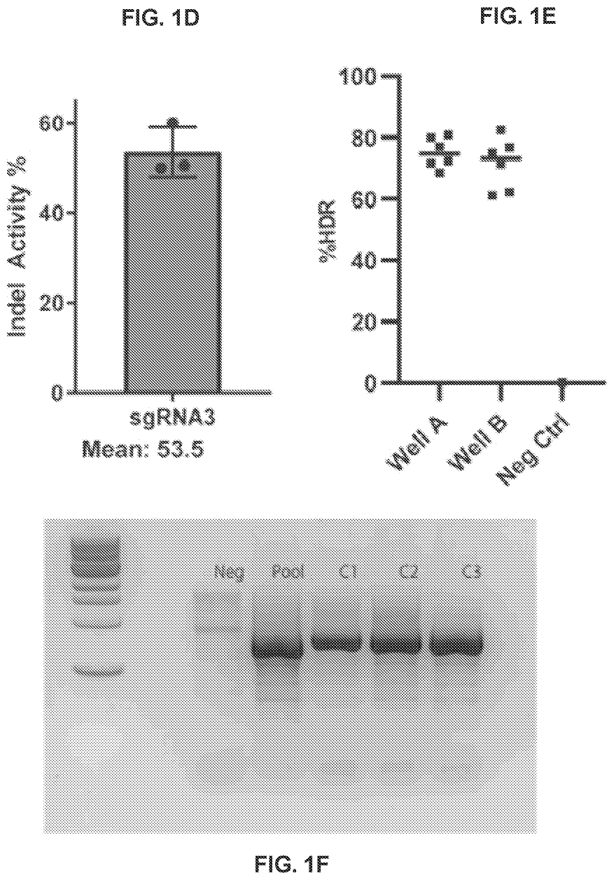

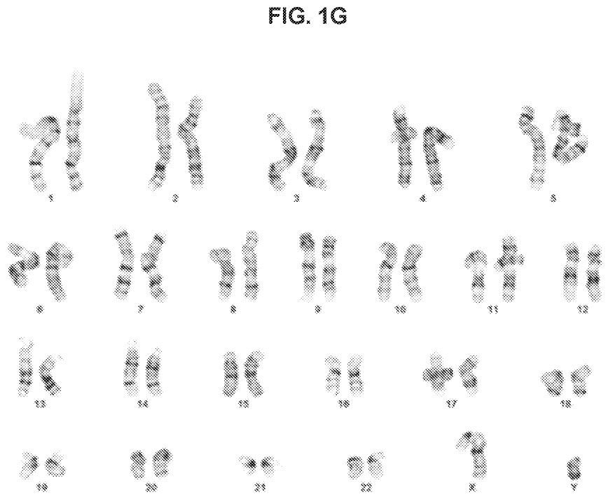

“RAG2” or “Recombination-activating Gene 2” refers to a gene encoding the “RAG2” protein, which is involved in V(D)J recombination during B and T cell development. The RAG2 protein forms a complex with RAG1, and the complex can induce double stranded breaks at recombination signal sequences. The RAG2 cDNAs used in the present methods are typically full-length (e.g., comprising exon 3). Integration of the cDNA using the present methods allow cells of the patient to express functional RAG2 protein and thus restore protein activity in patients. The accession number for the human RAG2 gene is NCBI Gene ID 5897, and for the encoded protein it is UniProt P55895. Codon-optimized (or “codon diverged”) versions of the RAG2 cDNA, comprising about 72% or 76% sequence homology to the endogenous, wild-type gene, are shown as SEQ ID NOS:5 and 6, respectively. The present methods can be used with any patient with RAG2 deficiency, with any RAG2 mutation, so long that the RAG2 locus retains a functional promoter and potentially other regulatory elements such that the integrated cDNA is expressed in cells from the patient and restores or ameliorates the decrease in RAG2 resulting from the mutation. As shown in FIG. 1C, the RAG2 gene comprises two initial “exons” that comprise 5′ UTRs but do not comprise any coding sequence. The coding sequence is entirely present in what is labeled “exon 3.” However, as exon 3 is the first exon to comprise a coding sequence, it is also at times referred to as exon 1. It is this exon (“exon 3” or “exon 1”, comprising the coding sequence and therefore comprising the translation start site) that is targeted by sgRNAs in particular embodiments of the present methods.

RAG2 deficiency can cause severe combined immunodeficiency disorder (SCID), e.g., Omenn syndrome (OS), an autosomal recessive form of SCID characterized by, e.g., erythroderma, desquamation, failure to thrive, lymphadenopathy, and other features, and leaving patients highly susceptible to infection. OS is associated with low IgG. IgA, and IgM, and the virtual absence of B cells. Other RAG2 mutations can lead to less severe, non-SCID RAG2 deficiency conditions, which are often associated with autoimmunity. In RAG2-associated SCID, the adaptive immune cells are unable to properly assemble functional antigen-specific receptors of the immunoglobulin (BCR) and T-cell (TCR) RAG2 functions as part of the complex required for V(D)J recombination activity, which is essential during lymphocyte development and antigen recognition. Diseases associated with RAG2 deficiency include: typical RAG2-SCID. Omenn syndrome (OS), atypical RAG2-SCID, expansion of γδ-T cells and granulomatous inflammation and/or autoimmunity. The present methods use adeno-associated viral vector of serotype 6 (AAV-6) to deliver a codon-optimized RAG2 (RAG2co) therapeutic transgene at the endogenous RAG2 gene translation initiation site in, e.g., human iPSCs or HSPCs. This approach is designed to correct all the pathogenic mutations that span the entire coding region up to an including the 3′ untranslated region. Any of a variety of mutations in RAG2, including missense mutations, nonsense mutations, insertions, deletions, and splicing mutations, can prevent the expression of functional encoded protein. The present methods can compensate for the deficiencies caused by such RAG2 mutations in patients, regardless of the nature of location of the mutations.

The term “treating” or “treatment” refers to any one of the following: ameliorating one or more symptoms of a disease or condition (e.g., RAG2 deficiency); preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

As used herein, the terms “subject”. “individual” or “patient” refer, interchangeably, to a warm-blooded animal such as a mammal. In particular embodiments, the term refers to a human. A subject may have, be suspected of having, or be predisposed to, RAG2 deficiency as described herein. The term also includes livestock, pet animals, or animals kept for study, including horses, cows, sheep, poultry, pigs, cats, dogs, zoo animals, goats, primates (e.g. chimpanzee), and rodents. A “subject in need thereof” refers to a subject that has one or more symptoms of RAG2 deficiency, that has received a diagnosis of RAG2 deficiency, that is suspected of having or being predisposed to RAG2 deficiency, that shows a deficiency of functional RAG2 or a polypeptide encoded by RAG2 as described herein, or that is thought to potentially benefit from increased expression of RAG2 as described herein.

An “effective amount” refers to an amount of a compound or composition, as disclosed herein effective to achieve a particular biological, therapeutic, or prophylatic result. Such results include, without limitation, the treatment of a disease or condition disclosed herein as determined by any means suitable in the art.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R). Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(sec, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the N-terminal residue, which is numbered 1, in an unmodified wild-type polypeptide sequence.

As used in herein, the terms “identical” or percent “identity.” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, 99.5%, 99.7%, 99.9%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 532, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

A “homologous repair template” refers to a polynucleotide sequence that can be used to repair a double stranded break (DSB) in the DNA, e.g., a CRISPR/Cas9-mediated break at the RAG2 locus as induced using the herein-described methods and compositions. The homologous repair template comprises homology to the genomic sequence surrounding the DSB, i.e., comprising RAG2 homology arms as described herein. In some embodiments, two distinct homologous regions are present on the template, with each region comprising at least 50, 100, 200, 300, 400, 500, 600, 700, 800, 900 or more nucleotides or more of homology with the corresponding genomic sequence. In particular embodiments, the templates comprise two homology arms comprising about 500 nucleotides of homology extending from either site of the sgRNA target site. The repair template can be present in any form, e.g., on a plasmid that is introduced into the cell, as a free floating doubled stranded DNA template (e.g., a template that is liberated from a plasmid in the cell), or as single stranded DNA. In particular embodiments, the template is present within a viral vector, e.g., an adeno-associated viral vector such as AAV6. The templates of the present disclosure also comprise a full-length, codon-optimized RAG2 cDNA, as well as, typically, a polyadenylation signal such as from bovine growth hormone.

As used herein, “homologous recombination” or “HR” refers to insertion of a nucleotide sequence during repair of double-strand breaks in DNA via homology-directed repair mechanisms. This process uses a “donor template” or “homologous repair template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems. In particular embodiments. HR involves double-stranded breaks induced by CRISPR-Cas9. In further embodiments, the CRISPR-Cas9 comprises high-fidelity Cas9 variants having improved on-target specificity and reduced off-target activity. Examples of high-fidelity Cas9 variants include but are not limited to those described in PCT Publication Nos. WO/2018/068053 and WO/2019/074542, each of which is herein incorporated by reference in its entirety.

As used herein, “functional RAG2 cDNA” refers to cDNA encoding a RAG2 protein having similar or equivalent protein function as wild-type RAG2 protein (UniProt P55895), which is referred to herein as “functional RAG2 protein.” In some embodiments, functional RAG2 protein has at least 50%, 55%, 60%, 65%, 70%, 75%, 80%, 85%, 90%, 95%, 97%, 99%, 99.5%, 99.7%, 99.9% or 100% of the function of wild-type RAG2 protein, as determined by any method known in the art for assessing RAG2 protein function.

### 4. CRISPR/Cas Systems Targeting the RAG2 Locus

The present disclosure provides methods and compositions for integrating functional RAG2 cDNAs into the endogenous RAG2 locus in cells from a subject with a RAG2 deficiency. In particular embodiments, the cells are induced pluripotent stem cells (iPSCs) or hematopoietic stem and progenitor cells (HSPCs). The cells can be modified using the methods described herein and then reintroduced into the subject, wherein the expression of the cDNA in the modified cells in vivo can restore protein activity and, consequently, V(D)J-recombination that is missing or deficient in the subject.

The present disclosure is based in part on the identification of CRISPR guide sequences that specifically and effectively direct the cleavage of RAG2, e.g., within exon 3 of RAG2, e.g., close to the translation start site, by RNA-guided nucleases such as Cas9. In particular embodiments, the methods involve the introduction of ribonucleoproteins (RNPs) comprising an sgRNA targeting RAG2 and Cas9, as well as a template DNA molecule comprising RAG2 homology arms flanking a functional, codon-optimized RAG2 cDNA. Using the present methods, high rates of targeted integration at the RAG2 locus (e.g., 39.8%+0.27% or more of iPSCs) and expression of the cDNA can be achieved, with the result that the modified cells can be differentiated in vitro into human embryonic mesodermal progenitor (hEMP) cells and then differentiated into T cells, e.g., using a 3D artificial thymic organoid (ATO) system. Modified iPSC's or HSPCs can be used for transplantation and long-term engraftment of the modified cells, leading to a reduction or elimination of symptoms caused by the RAG2 protein deficiency.

sgRNAs

The single guide RNAs (sgRNAs) used in the present methods target the RAG2 locus, sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence comprising homology (or complementarity) to a target DNA sequence at the RAG2 locus, and a constant region that mediates binding to Cas9 or another RNA-guided nuclease. The sgRNA can target any sequence within RAG2 adjacent to a PAM sequence. In some embodiments, the target sequence is within exon 3 of RAG2, e.g., close to the translation start site of the RAG2 gene, such that the promoter and upstream regulatory sequences are retained and drive expression of the inserted cDNA similarly to the endogenous gene. In particular embodiments, the target sequence of the sgRNA comprises one of the sequences shown as SEQ ID NO: 1 to SEQ ID NO:4, or a sequence having, e.g., at least 90%, 91%, 92%, 93%, 94%, 95%, 96%, 97%, 98%, 99%, or more identity to, e.g., comprising 1, 2, 3 or more nucleotide substitutions, additions or subtractions relative to any one of SEQ ID NO:1 to SEQ ID NO:4. In particular embodiments, the sgRNA comprises the sequence shown as SEQ ID NO: 1.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or 15-25, 18-22, or 19-21 nucleotides in length, and shares homology with a targeted genomic sequence, in particular at a position adjacent to a CRISPR PAM sequence. The sgRNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity. e.g., with 1-4 mismatches with the target DNA sequence).

Each sgRNA also includes a constant region that interacts with or binds to the site-directed nuclease, e.g., Cas9. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g. 90, 91, 92, 93, 94, 95, 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the tracrRNA provides a binding scaffold for the Cas nuclease.

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3′ phosphorothiate internucleotide linkages, 2′-O-methyl-3′-phospholactate modifications, 2′-fluoro-pyrimidines. S-constrained ethyl sugar modifications, or others, at one or more nucleotides. In particular embodiments, the sgRNAs comprise 2′-O-methyl-3′-phosphorothioate (MS) modifications at one or more nucleotides (sec, e.g., Hendel et al. (2015) Nat. Biotech. 33(9):985-989, the entire disclosure of which is herein incorporated by reference). In particular embodiments, the 2′-O-methyl-3′-phosphorothioate (MS) modifications are at the three terminal nucleotides of the S′ and 3′ ends of the sgRNA.

The sgRNAs can be obtained in any of a number of ways. For sgRNAs, primers can be synthesized in the laboratory using an oligo synthesizer, e.g., as sold by Applied Biosystems. Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

### RNA-Guided Nucleases

Any CRISPR-Cas nuclease can be used in the method. i.e., a CRISPR-Cas nuclease capable of interacting with a guide RNA and cleaving the DNA at the target site as defined by the guide RNA. In some embodiments, the nuclease is Cas9 or Cpf1. In particular embodiments, the nuclease is Cas9. The Cas9 or other nuclease used in the present methods can be from any source, so long that it is capable of binding to an sgRNA as described herein and being guided to and cleaving the specific RAG2 sequence targeted by the targeting sequence of the sgRNA. In particular embodiments, the Cas9 is from Streptococcus pyogenes.

Also disclosed herein are CRISPR/Cas or CRISPR/Cpf1 systems that target and cleave DNA at the RAG2 locus. An exemplary CRISPR/Cas system comprises (a) a Cas (e.g., Cas9) or Cpf1 polypeptide or a nucleic acid encoding said polypeptide, and (b) an sgRNA that hybridizes specifically to RAG2, or a nucleic acid encoding said guide RNA. In some instances, the nuclease systems described herein further comprise a donor template as described herein. In particular embodiments, the CRISPR/Cas system comprises an RNP comprising an sgRNA targeting RAG2 and a Cas protein such as Cas9. In some embodiments, the Cas9 is a high fidelity (HiFi) Cas9 variant (see, e.g., Vakulskas et al. (2018) Nat. Med. 24: 1216-1224).

In addition to the CRISPR/Cas9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1). Acidaminococcus sp. Cpf1 (AsCpf1), and Lachnospiracede bacterium ND2006 Cpf1 (LbCpf1) systems. Any of the above CRISPR systems may be used to induce a single or double stranded break at the RAG2 locus to carry out the methods disclosed herein.

Introducing the sgRNA and Cas Protein into Cells

The sgRNA and nuclease can be introduced into a cell using any suitable method. e.g., by introducing one or more polynucleotides encoding the sgRNA and the nuclease into the cell, e.g., using a vector such as a viral vector or delivered as naked DNA or RNA, such that the sgRNA and nuclease are expressed in the cell. In some embodiments, one or more polynucleotides encoding the sgRNA, the nuclease or a combination thereof are included in an expression cassette. In some embodiments, the sgRNA, the nuclease, or both sgRNA and nuclease are expressed in the cell from an expression cassette. In some embodiments, the sgRNA, the nuclease, or both sgRNA and nuclease are expressed in the cell under the control of a heterologous promoter. In some embodiments, one or more polynucleotides encoding the sgRNA and the nuclease are operatively linked to a heterologous promoter. In particular embodiments, the sgRNA and nuclease are assembled into ribonucleoproteins (RNPs) prior to delivery to the cells, and the RNPs are introduced into the cell by, e.g., electroporation. RNPs are complexes of RNA and RNA-binding proteins. In the context of the present methods, the RNPs comprise the RNA-binding nuclease (e.g., Cas9) assembled with the guide RNA (e.g., sgRNA), such that the RNPs are capable of binding to the target DNA (through the gRNA component of the RNP) and cleaving it (via the protein nuclease component of the RNP). As used herein, an RNP for use in the present methods can comprise any of the herein-described guide RNAs and any of the herein-described RNA-guided nucleases.

Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkey's.

In some embodiments, the cell is an embryonic stem cell, a stem cell, a progenitor cell, a pluripotent stem cell, an induced pluripotent stem cell (iPSC), a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). In particular embodiments, the cell is an iPSC, e.g., an iPSC derived from a fibroblast obtained from the subject and reprogrammed by expression of reprogramming factors. e.g., by infection with a non-integrating Sendai virus vector kit allowing transient expression of reprogramming factors OCT4, SOX2, c-MYC, and KLF4. In particular embodiments, the cell is a CD34⁺ hematopoietic stem and progenitor cell (HSPC), e.g., a cord blood-derived (CB), peripheral blood-derived (PB), or bone marrow derived HSPC.

To avoid immune rejection of the modified cells when administered to a subject, the cells to be modified are preferably derived from the subject's own cells. Thus, preferably the mammalian cells are autologous cells from the subject to be treated with the modified cells. In some embodiments, however, the cells are allogeneic, i.e., isolated from an HLA-matched or HLA-compatible, or otherwise suitable, donor.

In some embodiments, cells are harvested from the subject and modified according to the methods disclosed herein, which can include selecting certain cell types, optionally expanding the cells and optionally culturing the cells, and which can additionally include selecting cells that contain the transgene integrated into the RAG2 locus. In particular embodiments, such modified cells are then reintroduced into the subject.

Further disclosed herein are methods of using said nuclease systems to produce the modified host cells described herein, comprising introducing into the cell (a) an RNP of the present disclosure that targets and cleaves DNA at the RAG2 locus, and (b) a homologous donor template or vector as described herein. Each component can be introduced into the cell directly or can be expressed in the cell by introducing a nucleic acid encoding the components of said one or more nuclease systems.

Such methods will target integration of the functional RAG2 cDNA at the endogenous RAG2 locus in a host cell ex vivo. Such methods can further comprise (a) introducing a donor template or vector into the cell, optionally after expanding said cells, or optionally before expanding said cells, and (b) optionally culturing the cell.

In some embodiments, the disclosure herein contemplates a method of producing a modified mammalian host cell, the method comprising introducing into a mammalian cell: (a) an RNP comprising a Cas nuclease such as Cas9 and an sgRNA specific to the RAG2 locus, and (b) a homologous donor template or vector as described herein. The disclosure further contemplates a mammalian host cell composition, wherein the mammalian host cell comprises: (a) an RNP comprising a Cas nuclease such as Cas9 and an sgRNA specific to the RAG2 locus, and (b) a homologous donor template or vector as described herein.

In any of these methods, the nuclease can produce one or more single stranded breaks within the RAG2 locus, or a double stranded break within the RAG2 locus. In these methods, the RAG2 locus is modified by homologous recombination with said donor template or vector to result in insertion of the transgene into the locus. The methods can further comprise (c) selecting cells that contain the transgene integrated into the RAG2 locus.

In some embodiments, 153 (Canny et al. (2018) Nat Biotechnol 36:95) is introduced into the cell in order to promote integration of the donor template by homology directed repair (HDR) versus integration by non-homologous end-joining (NHEJ). For example, an mRNA encoding i53 or a recombinant i53 protein can be introduced into the cell, e.g., by electroporation at the same time as an sgRNA-Cas9 RNP. The sequence of i53 can be found, inter alia, at www.addgene.org/92170/sequences/.

Techniques for insertion of transgenes, including large transgenes, capable of expressing functional proteins, including enzymes, cytokines, antibodies, and cell surface receptors are known in the art (See, e.g. Bak and Porteus. Cell Rep. 2017 Jul. 18; 20(3): 750-756 (integration of EGFR); Kanojia et al., Stem Cells. 2015 October; 33(10):2985-94 (expression of anti-Her2 antibody); Eyquem et al., Nature. 2017 Mar. 2:543(7643): 113-117 (site-specific integration of a CAR); O'Connell et al., 2010 PLOS ONE 5(8): e12009 (expression of human IL-7); Tuszynski et al., Nat Med. 2005 May; 11(5):551-5 (expression of NGF in fibroblasts); Sessa et al., Lancet. 2016 Jul. 30; 388(10043):476-87 (expression of arylsulfatase A in ex vivo gene therapy to treat MLD); Rocca et al., Science Translational Medicine 25 Oct. 2017: Vol. 9, Issue 413, eaaj2347 (expression of frataxin); Bak and Porteus, Cell Reports, Vol. 20. Issue 3, 18 Jul. 2017, Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is herein incorporated by reference in its entirety.

### Homologous Repair Templates

The RAG2 cDNA to be integrated, which is comprised by a polynucleotide or donor construct, can be any functional, codon-optimized RAG2 cDNA whose expression in cells can restore or improve protein levels in RAG2 deficiency patients and thereby allow normal, or clinically beneficial, V(D)J recombination and consequently T and/or B cell development and function. In particular embodiments, the cDNA is integrated, e.g., at the translational start site of the endogenous RAG2 locus, using a template comprising a RAG2 cDNA. In such cases, the cDNA is expressed under the control of the endogenous RAG2 promoter and other regulatory elements.

In particular embodiments, the RAG2 cDNA in the homologous repair template is codon-optimized, e.g., comprises at least 70%, 71%, 72%, 73%, 74%, 75%, 76%, 77%, 78%, 79%, 80%, 85%, 90%, 95%, or more homology to the wild-type RAG2 cDNA. In a particular embodiment, the RAG2 cDNA comprises about 72% or 76% homology to the wild-type RAG2 cDNA. In a particular embodiment, the RAG2 cDNA comprises the codon-optimized sequence shown as SEQ ID NO: 5 or SEQ ID NO:6, or a derivative or fragment of SEQ ID NO: 5 or SEQ ID NO:6, e.g., a sequence having about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.7%, 99.9% or greater identity to SEQ ID NO: 5 or SEQ ID NO:6 or to a fragment thereof.

In particular embodiments, the template further comprises a polyA sequence or signal, e.g., a bovine growth hormone polyA sequence, at the 3′ end of the cDNA. In particular embodiments, a Woodchuck Hepatitis Virus Posttranscriptional Regulatory Element (WPRE) is included within the 3′UTR of the template, e.g., between the 3′ end of the RAG2 coding sequence and the 5′ end of the polyA sequence, so as to increase the expression of the cDNA. Any suitable WPRE sequence can be used; See, e.g., Zufferey et al. (1999) J. Virol. 73(4):2886-2892; Donello, et al. (1998). J Virol 72: 5085-5092; Loeb, et al. (1999). Hum Gene Ther 10: 2295-2305; the entire disclosures of which are herein incorporated by reference.

In particular embodiments, the cDNA (or cDNA and polyA signal) is flanked in the template by RAG2 homology regions. For example, an exemplary template can comprise, in linear order: a first RAG2 homology region, a RAG2 cDNA, a polyA sequence such as a bovine growth hormone polyadenylation sequence (bGH-PolyA) or a rabbit beta-globin polyA sequence, and a second RAG2 homology region, where the first and second homology regions are homologous to the genomic sequences extending in either direction from the sgRNA target site. In particular embodiments, one of the homology regions comprises the sequence of nucleotides 1-447 of SEQ ID NO:7, or a contiguous portion thereof, or a sequence having 80%, 85%, 90%, 95%, 96%, 97%, 98%, 99%, 99.5%, 99.7%, 99.9% or greater identity to nucleotides 1-447 of SEQ ID NO:7, or a contiguous portion thereof. In particular embodiments, the other homology region comprises the sequence of nucleotides 2848-3247 of SEQ ID NO:7, or a contiguous portion thereof, or a sequence having 80%, 85%, 90%, 95%, 96%, 97%, 98%, 99%, or greater identity to nucleotides 2848-3247 of SEQ ID NO:7, or a contiguous portion thereof. The homology regions can be of any size, e.g., 100-1000 bp, 300-800 bp, 400-600 bp, or about 100, 200, 300, 400, 500, 600, 700, 800, 900, 1000, or more bp. In particular embodiments, the homology regions are about 400-500 bp in size.

In particular embodiments, the homologous repair template comprises the sequence shown as SEQ ID NO:7. In other embodiments, the homologous repair template comprises a sequence having 80%, 85%, 90%, 95%, 96%, 97%, 98%, 99%, 99.5%, 99.7%, 99.9% or greater identity to SEQ ID NO:7 or a contiguous portion thereof.

Any suitable method can be used to introduce the polynucleotide, or donor construct, into the cell. In particular embodiments, the polynucleotide is introduced using a recombinant adeno-associated viral vector (rAAV). For example, the rAAV can be from serotype 1 (e.g., an rAAV1 vector), 2 (e.g., an rAAV2 vector), 3 (e.g., an rAAV3 vector), 4 (e.g., an rAAV4 vector), 5 (e.g., an rAAV5 vector), 6 (e.g., an rAAV6 vector), 7 (e.g., an rAAV7 vector), 8 (e.g., an rAAV8 vector), 9 (e.g., an rAAV9 vector), 10 (e.g., an rAAV10 vector), or 11 (e.g., an rAAV11 vector). In particular embodiments, the vector is an rAAV6 vector. In some instances, the donor template is single stranded, double stranded, a plasmid or a DNA fragment. In some instances, plasmids comprise elements necessary for replication, including a promoter and optionally a 3′ UTR.

Further disclosed herein are vectors comprising (a) one or more nucleotide sequences homologous to the RAG2 locus, and (b) a RAG2 cDNA as described herein. The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector.

In some embodiments, the targeting construct comprises: (1) a viral vector backbone, e.g. an AAV backbone, to generate virus, (2) arms of homology to the target site of at least 200 bp but ideally at least 400 bp on each side to assure high levels of reproducible targeting to the site (see, Porteus, Annual Review of Pharmacology and Toxicology, Vol. 56:163-190 (2016); which is hereby incorporated by reference in its entirety); (3) a RAG2 cDNA encoding a functional protein and capable of expressing the functional protein, a polyA sequence, and optionally a WPRE element; and optionally (4) an additional marker gene to allow for enrichment and/or monitoring of the modified host cells. Any AAV known in the art can be used. In some embodiments the primary AAV serotype is AAV6. In some embodiments, the vector, e.g., rAAV6 vector, comprising the donor template is from about 1-2 kb, 2-3 kb, 3-4 kb, 4-5 kb, 5-6 kb, 6-7 kb, 7-8 kb, or larger.

In some embodiments, a viral vector, e.g., AAV6 vector, is transduced at a multiplicity of infection (MOI) of, e.g., about 1×10³, 5×10³, 1χ10⁴, 5×10⁴, 1×10⁵, 2.5×10⁵, 5×10⁵, 1×10⁶, between 1×10⁴ and 1×10⁵ viruses (vector genomes) per cell, or between 1×10⁵ and 1×10⁶ viruses (vector genomes) per cell.

In some embodiments, the vector comprises a marker gene, e.g., to allow selection of genetically modified cells. Suitable marker genes are known in the art and include Myc, HA. FLAG, GFP, truncated NGFR, truncated EGFR, truncated CD20, truncated CD19, as well as antibiotic resistance genes. In some embodiments, the homologous repair template and/or vector (e.g., AAV6) comprises an expression cassette comprising a coding sequence for truncated nerve growth factor receptor (INGFR), operably linked to a promoter such as the Ubiquitin C promoter.

In any of the preceding embodiments, the donor template or vector comprises a nucleotide sequence homologous to a fragment of the RAG2 locus, optionally to the sequences shown as nucleotides 1-447 or 2848-3247 of SEQ ID NO:7 or contiguous portions thereof, wherein the nucleotide sequence is at least 85%, 88%, 90%, 92%, 95%, 98%, 99%, 99.5%, 99.7%, or 99.9% identical to at least 200, 250, 300, 350, 400, 450, 500, or more consecutive nucleotides of the RAG2 locus, e.g., to nucleotides 1-447 and 2848-3247 of SEQ ID NO:7.

The inserted construct can also include other safety switches, such as a standard suicide gene in the locus (e.g. iCasp9) in circumstances where rapid removal of cells might be required due to acute toxicity. The present disclosure provides a robust safety switch so that any engineered cell transplanted into a body can be eliminated, e.g., by removal of an auxotrophic factor. This is especially important if the engineered cell has transformed into a cancerous cell.

The present methods allow for the efficient integration of the donor template at the endogenous RAG2 locus. In some embodiments, the present methods allow for the insertion of the donor template in, e.g., 20%, 25%, 30%, 35%, 40%, or more cells, e.g., iPSCs or HSPCs derived from cells obtained from an individual with a RAG2 deficiency. The methods also allow for high levels of expression of RAG2 protein in cells, e.g., iPSCs or HSPCs from an individual with a RAG2 deficiency, with an integrated RAG2 cDNA. The methods also allow for the edited cells to differentiate, e.g., into viable hEMP cells, T cells (at various stages, e.g., pro-T: CD34⁺ CD7⁺ CD5⁻; pre-T: CD7⁺ CD5⁺ CD1a⁺; CD4⁺ CD8⁺ double positive (DP) cells; and CD3⁺ TCRαβ⁺ or CD3⁺ TCRγδ T cells), B cells (e.g., CD19⁺ or CD20⁺ IgM⁺), or NK cells, and to undergo V(D)J recombination, and to be reintroduced into the subject for the treatment of the RAG2 deficiency.

### 5. Methods of Treatment

Following the integration of the cDNA into the genome of the cell, e.g., iPSC or HSPC, and confirming expression of the encoded protein, a plurality of modified cells can be reintroduced into the subject, such that they can repopulate and differentiate into, e.g. T cells and B cells, and due to the expression of the integrated cDNA, can improve one or more abnormalities or symptoms in the subject with the RAG2 deficiency. In some embodiments, the cells are expanded, selected, and/or induced to undergo differentiation, prior to reintroduction into the subject.

Disclosed herein, in some embodiments, are methods of treating RAG2 deficiency in an individual in need thereof, the method comprising providing to the individual a protein replacement therapy using the genome modification methods disclosed herein. In some instances, the method comprises administering to the individual a modified host cell comprising a functional RAG2 cDNA, integrated at the RAG2 locus, wherein said modified host cell expresses the encoded protein which is otherwise deficient in the individual, thereby treating the RAG2 deficiency in the individual. The RAG2 deficiency can involve any condition or disease resulting from a decrease in RAG2 activity or level, e.g., as a result of a mutation causing a loss of or decrease in one or more aspect of RAG2 expression, activity, or stability, so long that the endogenous RAG2 promoter and regulatory elements are still present and capable of driving the expression of the RAG2 cDNA integrated at the RAG2 locus. In some embodiments, the RAG2 deficiency is a typical RAG2 SCID (severe combined immunodeficiency). In some embodiments, the RAG2 deficiency is an atypical RAG2 SCID. In some embodiments, the RAG2 deficiency is a non-SCID condition. In some embodiments, the RAG2 deficiency is Omenn Syndrome. In some embodiments, the RAG2 deficiency involves expansion of gamma delta T cells, granulomatous inflammation, and/or autoimmunity. In some embodiments, the modified host cell is modified ex vivo.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals. In some embodiments, the modified cells of the pharmaceutical composition are autologous to the individual in need thereof. In other embodiments, the modified cells of the pharmaceutical composition are allogeneic to the individual in need thereof.

In some embodiments, a pharmaceutical composition comprising a modified autologous host cell (e.g., iPSC or HSPC) as described herein is provided. The modified autologous host cell is genetically engineered to comprise an integrated RAG2 cDNA at the RAG2 locus. In particular embodiments, a functional codon-optimized RAG2 cDNA is integrated into the endogenous RAG2 locus. In particular embodiments, the functional codon-optimized RAG2 cDNA that is integrated into the host cell genome is expressed under control of the native RAG2 promoter sequence. In some embodiments, the pharmaceutical composition comprises a plurality of the modified host cells, and further comprises unmodified host cells and/or host cells that have undergone nuclease cleavage resulting in INDELS at the RAG2 locus but not integration of the RAG2 cDNA. In some embodiments, the pharmaceutical composition is comprised of at least 5% of the modified host cells comprising an integrated RAG2 cDNA. In some embodiments, the pharmaceutical composition is comprised of about 9% to 50% of the modified host cells comprising an integrated RAG2 cDNA. In some embodiments, the pharmaceutical composition is comprised of about 5% to 80% of the modified host cells comprising an integrated RAG2 cDNA, or 5% to 75%, 5% to 70%, 5% to 65%, 5% to 60%, 5% to 55%, or 5% to 50% of the modified host cells comprising an integrated RAG2 cDNA. In some embodiments, the pharmaceutical composition is comprised of about 10% to 80% of the modified host cells comprising an integrated RAG2 cDNA, or 10% to 75%, 10% to 70%, 10% to 65%, 10% to 60%, 10% to 55%, or 10% to 50% of the modified host cells comprising an integrated RAG2 cDNA In some embodiments, the pharmaceutical composition is comprised of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50% or more of the modified host cells comprising an integrated RAG2 cDNA. The pharmaceutical compositions described herein may be formulated using one or more excipients to, e.g.: (1) increase stability; (2) alter the biodistribution (e.g., target the cells to specific tissues or cell types); and/or (3) alter the release profile.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g., the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous, interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously. In certain embodiments, the composition may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

In some embodiments, a subject will undergo a conditioning regimen before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318 (5854) Science 1296-9 (2007); Palchaudari et al., 34 (7) Nature Biotechnology 738-745 (2016); Chhabra et al., 10:8 (351) Science Translational Medicine 351ra105 (2016)) and CAR T-mediated conditioning (see, e.g., Arai et al., 26 (5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing the RAG2 deficiency. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the disorder being treated and the severity of the disorder; the activity of the specific payload employed; the specific composition employed; the age, body weight, general health, sex and diet of the patient; the time of administration, route of administration; the duration of the treatment; drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more modified cells to the subject, or any amount sufficient to obtain the desired therapeutic or prophylactic, effect. The desired dosage of the modified host cell pharmaceutical compositions of the present disclosure may be administered one time or multiple times. In some embodiments, delivery of the modified host cell to a subject provides a therapeutic effect for at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, cither sequentially or concurrently. In general, each agent will be administered at a dose and/or on a time schedule determined for that agent.

Use of a modified mammalian host cell according to the present disclosure for treatment of RAG2 deficiency is also encompassed by the disclosure.

The present disclosure also contemplates kits comprising compositions or components of the present disclosure, e.g., sgRNA, Cas9, RNPs, i53, and/or homologous templates, as well as, optionally, reagents for, e.g., the introduction of the components into cells. The kits can also comprise one or more containers or vials, as well as instructions for using the compositions in order to modify cells and treat subjects according to the methods described herein.

### 6. Examples

The present methods and compositions will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. Gene Editing Rescues In Vitro T Cell Development of RAG2-Deficient Induced Pluripotent Stem Cells in an Artificial Thymic Organoid System

Severe combined immune deficiency (SCID) comprises an array of inherited genetic defects that affect the development of T lymphocytes (and, in some cases, also B and/or NK cells), thereby compromising adaptive immune responses. Patients with SCID are highly susceptible to serious infections from birth and inevitably die within the first years of life unless treated with allogeneic hematopoietic stem cell trans-plantation (HSCT). The recombination-activating genes 1 and 2 (RAG1 and RAG2) proteins initiate the process of V(D)J recombination that gives rise to a diverse repertoire of T and B cell receptors (TCRs. BCRs), thereby allowing recognition of antigens and adaptive immune responses [1]. Mutations in RAG1 or RAG2 can result in various clinical phenotypes [2]. Functionally null mutations cause a complete arrest of T and B cell development, resulting in T− B− NK+ SCID, whereas hypomorphic variants that allow residual RAG function are partially permissive to T (and in some cases, B) cell develop-ment and often manifest with immune dysregulation as a re-sult of faulty negative selection of self-reactive cells, in addi-tion to infections [3].

Currently, the only definitive cure for RAG deficiency is represented by allogeneic HSCT; however, this treatment comes with an array of possible complications including graft vs. host disease and transplant-related toxicities. Furthermore, challenges exist in finding matched donors for select ethnic groups, and graft failure and incomplete immune reconstitution have been frequently reported, especially after uncondi-tioned haploidentical HSCT [4]. Previous preclinical attempts to correct RAG deficiency by gammaretrovirus- or lentivirus-mediated gene transfer in mice have led to controversial re-sults, reflecting inadequate and/or dysregulated expression of the RAG genes using heterologous promoters [5-11].

To circumvent some of these challenges, we have taken an ex vivo editing approach to engineer patient-derived induced pluripotent stem cells (iPSCs), by knocking-in a promoterless RAG2 cDNA at the endogenous locus, thereby maintaining epigenetically controlled expression of the RAG2 gene, while avoiding the need to tailor donor templates to correct patient-specific mutations [12-15]. To assess rescue of T-cell differentiation, patient-derived iPSCs were differentiated into human embryonic mesodermal progenitors (hEMPs) and then cultured in an artificial thymic organoid (ATO) system that includes a murine stromal cell line (MS5) engineered to express the human Notch Delta-like ligand 4 (DLL4) in serum-free medium enriched with lymphopoietic cytokines and growth factors. Progressive maturation of T-cell development was monitored by flow-cytometry and high throughput sequencing was used to analyze diversity of the T-cell repertoire. Results were benchmarked against what was observed with unedited patient-derived iPSCs and healthy donor iPSCs.

### Results

Derivation, Characterization and Gene Editing of Patient RAG2-Mutated iPSC Line

Fibroblasts from a patient with T⁻ B⁻ NK⁺ SCID due to a homozygous RAG2 null mutation (c.831T>A, predicted to cause p. Y277* premature termination) were reprogrammed to iPSCs by infection with a non-integrating Sendai virus vector kit allowing transient expression of reprogramming factors OCT4, SOX2, c-MYC and KLF4. RAG2-mutated, patient-derived iPSCs were expanded and subcloned. Quantitative real-time polymerase chain reaction (qRT-PCR) demonstrated robust expression of stemness and pluripotency genes (FIG. 1A), and G-banding showed an apparently normal karyotype (FIG. 1B).

Gene targeting at the RAG2 locus in patient-derived iPSCs was performed using CRISPR-Cas9 technology. All amino acids present in the RAG2 protein are encoded by exon 3. The exon 3 sequence was codon-optimized using IDT's codon optimization tool to allow for improved translational efficiency and to diverge the cargo's nucleotide sequence identity from that of the wild type locus to circumvent premature cross over during the homology directed repair of the Cas9-derived double stranded DNA break. This codon-optimized recoded RAG2 (r.RAG2) cDNA was cloned into adeno associated virus of serotype 6 (AAV6) vector and produced using 293T HEK cells and an iodixanol gradient for purification before tittering using ddPCR.

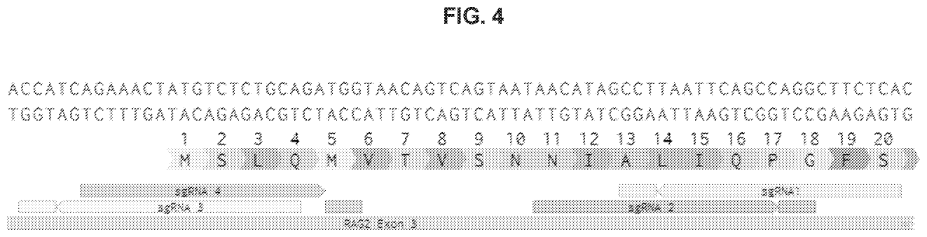

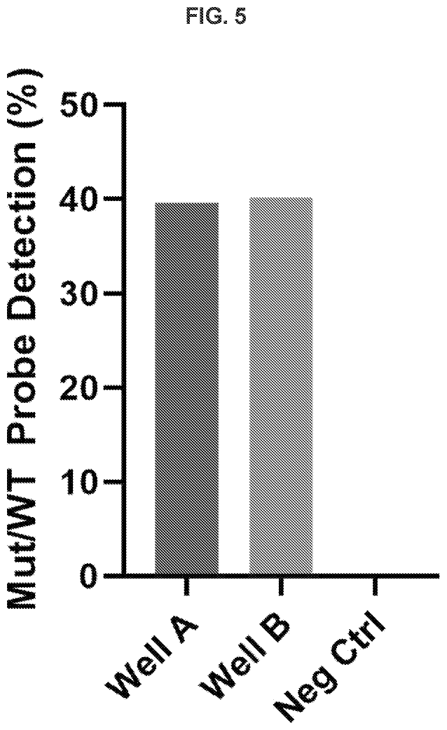

To target the RAG2 locus at exon 3, four single-guide RNAs were generated that recognize target DNA sequences around the translation initiation site (FIGS. 1C, 4). As a potential HSCT therapy, the designed sgRNAs were evaluated in umbilical cord blood (UCB)-derived hematopoietic stem cells for quantification of INDEL activity and cellular viability. SgRNA #3 was selected due to its proximity to the start codon (6 base pairs upstream of start site). In general, gRNAs with high INDEL activity have stimulated high gene targeting efficiencies [16]. Using MS modification and HPLC purification along with high fidelity spCas9 protein, previously optimized SpCas9/sgRNA molar ratios and electroporation conditions, an average bulk allele INDEL activity of 53.5% percent was observed in iPSCs (FIG. 1D). RAG2-mutated iPSCs were targeted with a high fidelity spCas9/sgRNA ribonucleoprotein (RNP) complex, with the donor repair template delivered via AAV6. Verification and quantification of the integration of the r.RAG2 cDNA into the endogenous locus was confirmed by ddPCR to be 39.87±0.27% (FIG. 5). This heterogeneous pool of cells was then enriched by sib-selection to contain targeted integration in approximately 80% of clones (FIG. 1E). Subsequent PCR of the knock-in junction sites spanning the genomic insertion locus of subcloned populations showed targeted integration (FIG. 1F). Karyotyping of an iPSC clone with homozygous integration of the r.RAG2 cDNA showed lack of cytogenetic aberrations (FIG. 1G). Paired mutation calling identified no putative variants occurring in regions (within 10 kb) identified as having BLAST similarity to our sgRNA. Patient derived iPSCs demonstrated expression of pluripotency markers, efficient targeted integration of the r.RAG2 cassette, and the genomic integrity to support subsequent differentiation studies.

### RAG2 Gene Editing Rescues Human T Cell Development In Vitro

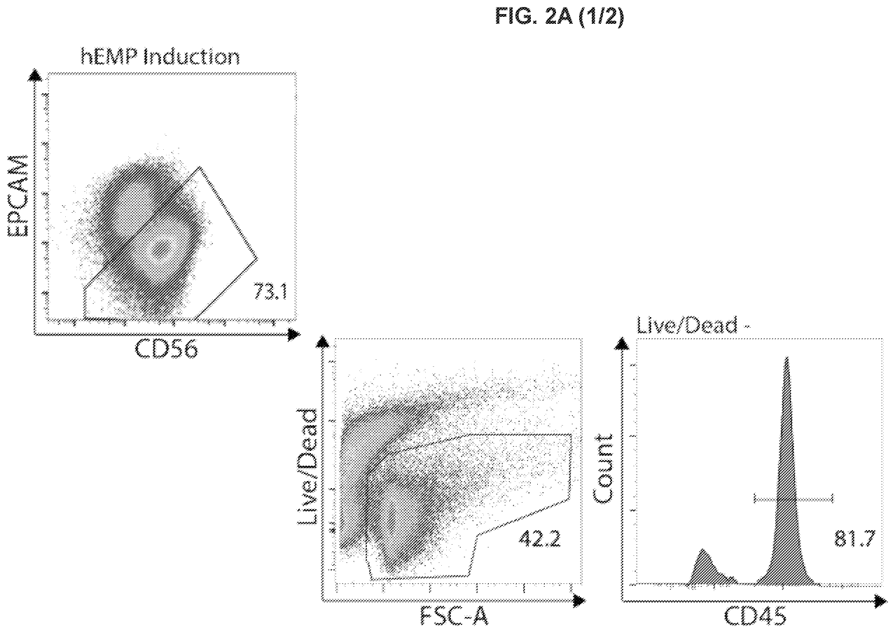

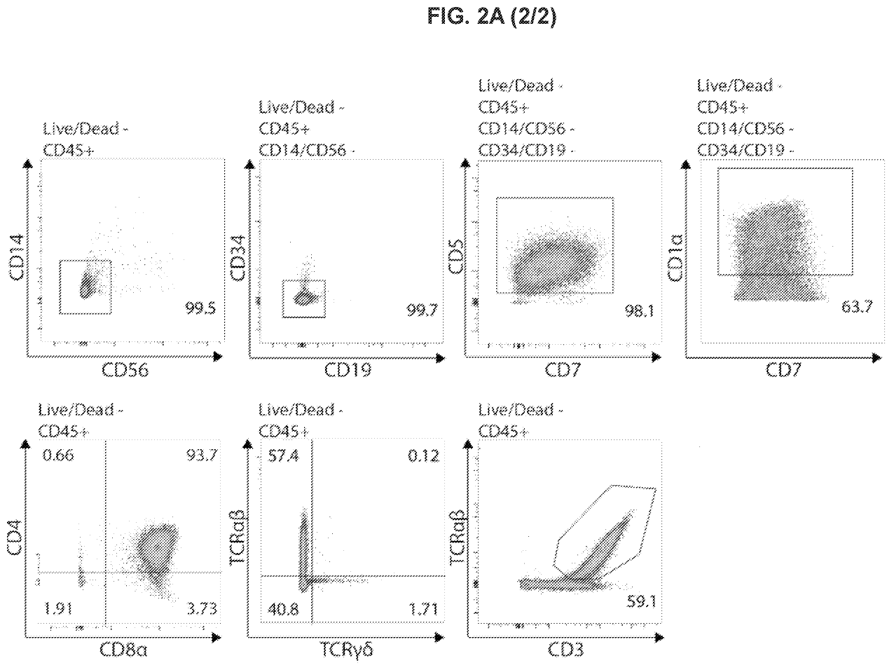

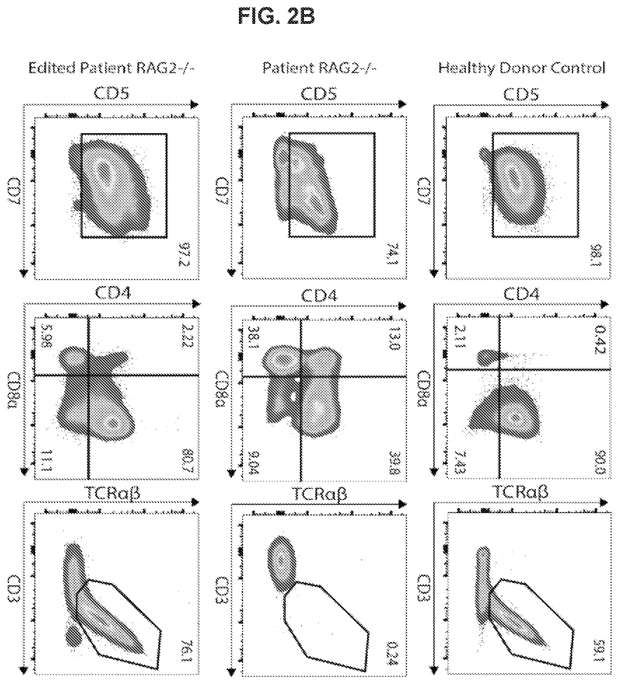

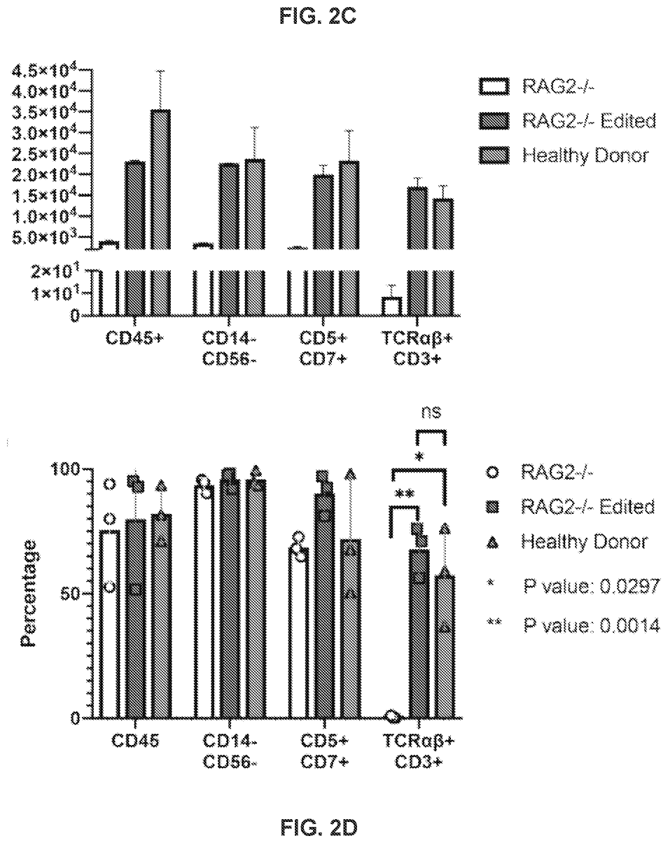

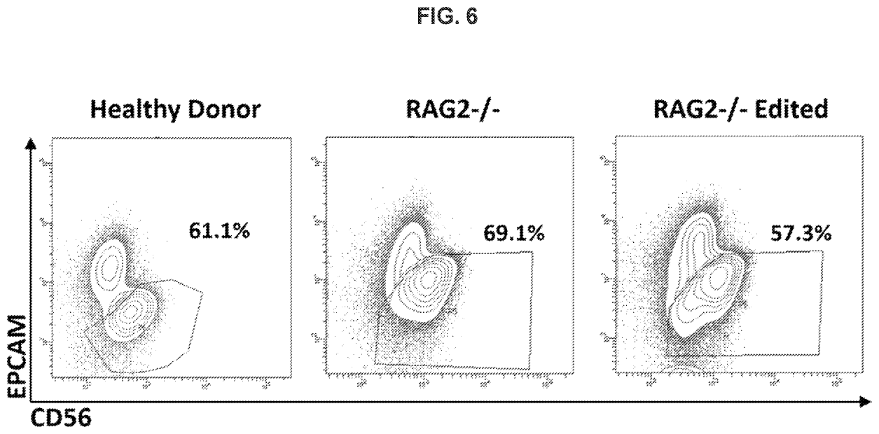

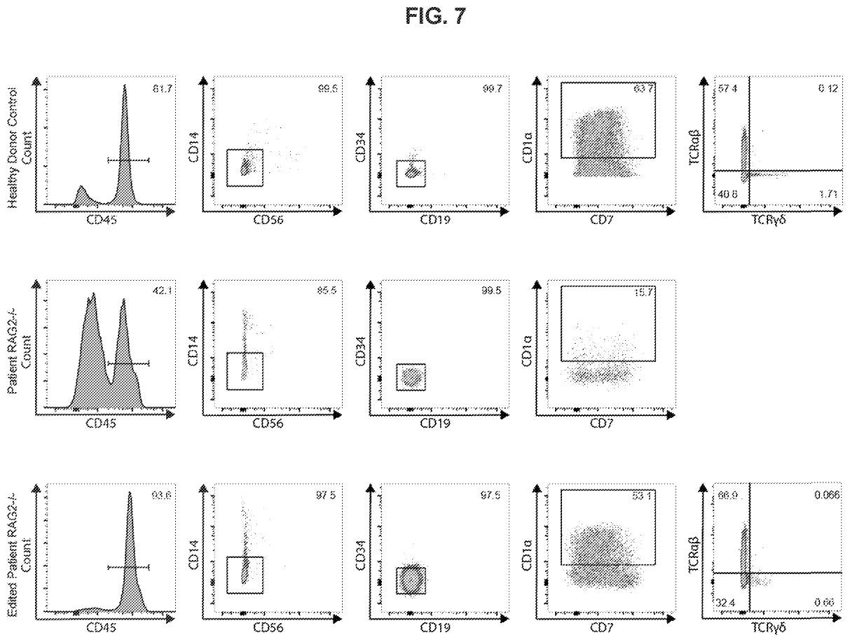

To assess whether targeted integration of the r.RAG2 cDNA at the endogenous locus rescues T cell development, we used a recently published method whereby iPSCs are differentiated into human embryonic mesodermal progenitor (hEMP) cells, and then differentiated into T cells in a 3D artificial thymic organoid (ATO) system [17]. In this platform, iPSC-derived hEMPs are co-cultured with the murine stromal cell line MS5 engineered to express the human Notch ligand DLL4 (MS5-hDLL4) in the presence of lymphopoietic cytokines and growth factors. Control-derived iPSCs, patient derived RAG2-mutated unedited, and RAG2 gene-edited iPSCs were successfully differentiated into CD56″ EPCAM-hEMPs (FIG. 6). Upon co-culture with MS5-hDLL4 in the ATO system, control cells showed progressive expression of markers corresponding to distinct stages of T-cell development (pro-T: CD34⁺ CD7⁺ CD5⁻; pre-T: CD7⁺ CD5⁺ CD1a⁺; CD4⁺ CD8⁺ double positive (DP) cells; and CD3⁺ TCRαβ+(or CD3⁺ TCRγδ⁺) T cells) (FIGS. 2A, 7). Few myeloid (CD14⁺) and NK (CD3⁻ CD56⁺) cells, and no CD19⁺ B cells were generated in the system from control-derived iPSCs (FIGS. 2A, 7). RAG2-mutated, unedited cells were able to progress through CD7⁺ CD5⁺ pre-T cell stage before becoming blocked at the DP stage, with lack of maturation to CD3⁺ TCRαβ⁺ (or CD3⁺ TCRγδ⁺) T cells (FIG. 2B). These are data are consistent with a severe impairment of V(D)J recombination and with the patient's immunological phenotype, as well as with the recent demonstration by our group that RAG deficiency in humans allows differentiation to DP cells but impedes development of CD3-TCRαβ⁺ cells [18]. In addition, a low cell yield per ATO was obtained during in vitro T-cell differentiation of unedited RAG2-deficient cells, suggestive of reduced viability from failure to progress past beta selection (FIG. 2C). By contrast, the RAG2 gene-edited, patient-derived line showed robust progression beyond the DP stage, with generation of CD3⁺ TCRαβ⁻ in a proportion that was comparable to that obtained during differentiation of normal donor iPSCs (FIGS. 2B, 7). Moreover, a similar number of patient-derived gene edited cells and control-derived cells were obtained throughout the various stages of T-cell differentiation, indicating rescue of cell viability (FIGS. 2C, 2D). These data indicate that this cDNA gene replacement strategy restores a functional RAG complex to developing T lymphocytes.

### Analysis of TCR Repertoire Diversity in Gene-Edited Cells

To further investigate the robustness of T cell development rescue, we analyzed the quality and identity of TCR rearrangements at the TCRB (TRB) and TCRα/TCRδ (TRA/TRD) loci by the high throughput immunoSEQ service offered by Adaptive Biotechnologies.

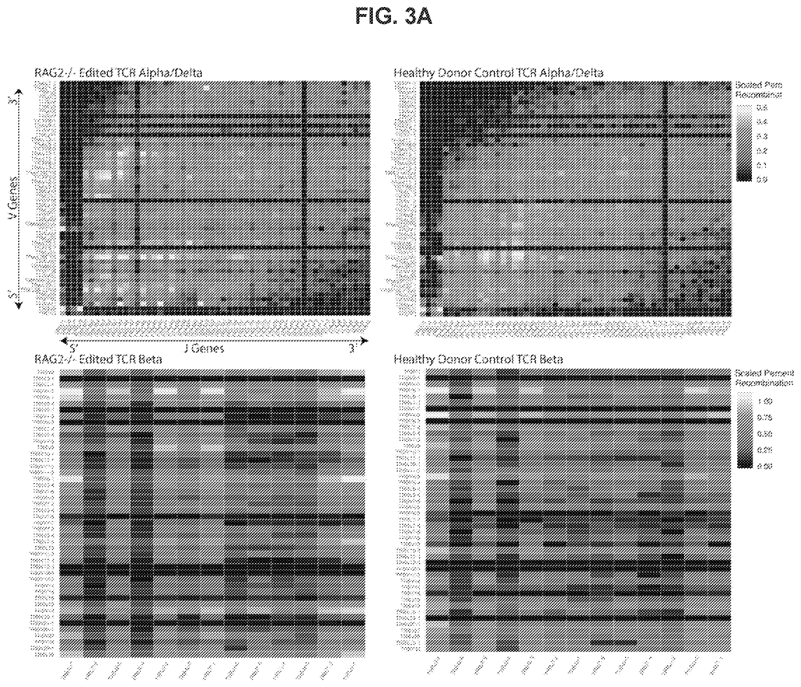

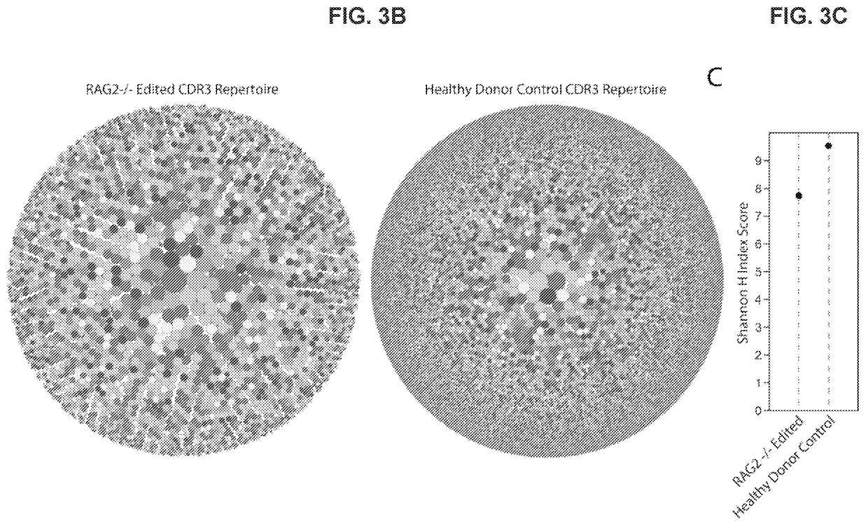

VJ recombination at the TRA locus is not entirely stochastic. Specifically, the most downstream TRAV and the most upstream TRAJ genes (located proximally to each other) are rearranged first, whereas rearrangement of the most distal genes occurs by sequential rounds of recombination in thymocytes that survive through the process [19, 20]. Prior work has demonstrated that mature T cells from patients with hypomorphic RAG mutations manifest an abnormal composition of the TRA repertoire, with reduced usage of the most distal TRAV and TRAJ genes [21]. This impairment was not observed in RAG2 deficient, gene-edited patient T cells. A polyclonal pattern of rearrangements at the TRB and TRA/TRD loci, and a similar pattern in the usage of TRAV genes, were observed in bulk CD3⁺ cells derived from healthy control iPSCs and from patient-derived RAG2-gene edited iPSCs that had been differentiated in the ATO system (FIG. 3A), suggesting robust rescue of VJ pairing in edited cells.

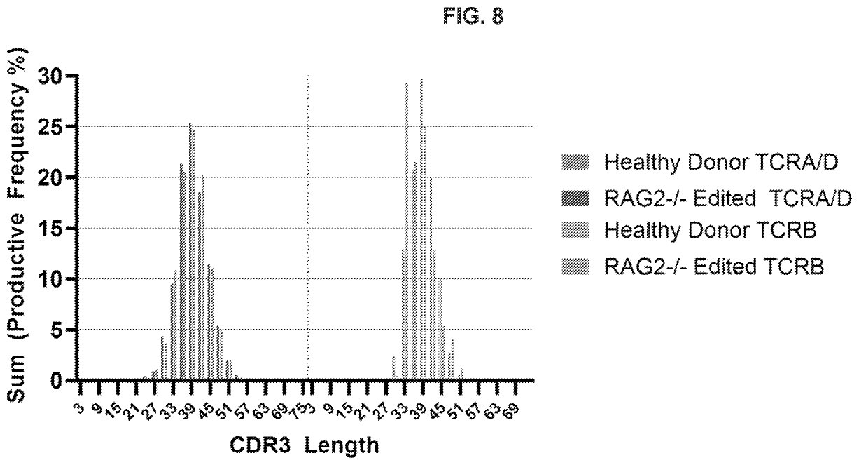

The complementary determining region 3 (CDR3) determines the specificity of the TCR to its cognate peptide MHC complex. A tree-map profile analyzing CDR3 identities revealed a polyclonal pattern of TCR CDR3 specificities, with a Shannon's H entropy index demonstrating a similar diversity of the CDR3 repertoire in sorted CD3⁺ cells derived from control and from RAG2 gene-edited iPSCs (FIGS. 3B-C). Furthermore, virtual spectratyping revealed similar CDR3 lengths in CD3⁺ cells from patient-derived gene-edited iPSCs and control iPSCs (FIG. 8).

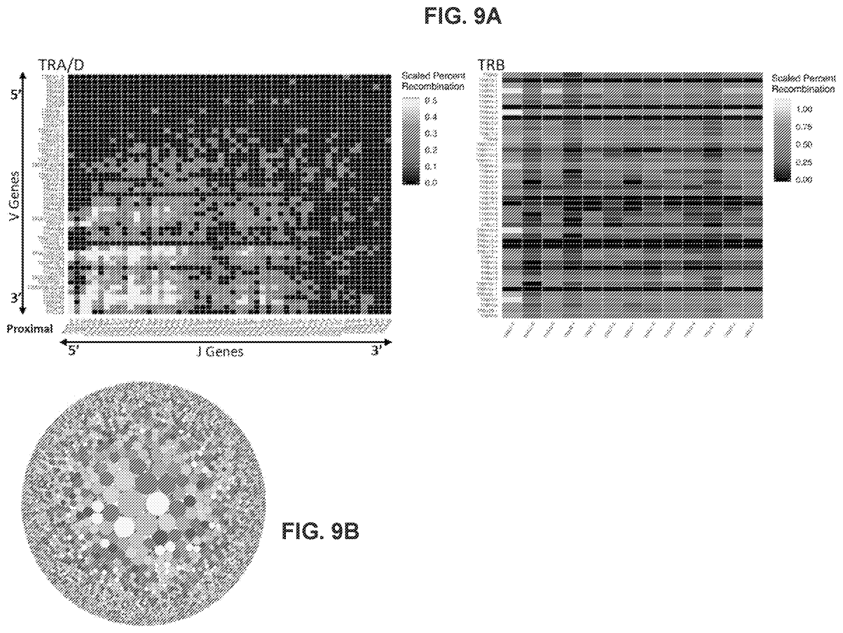

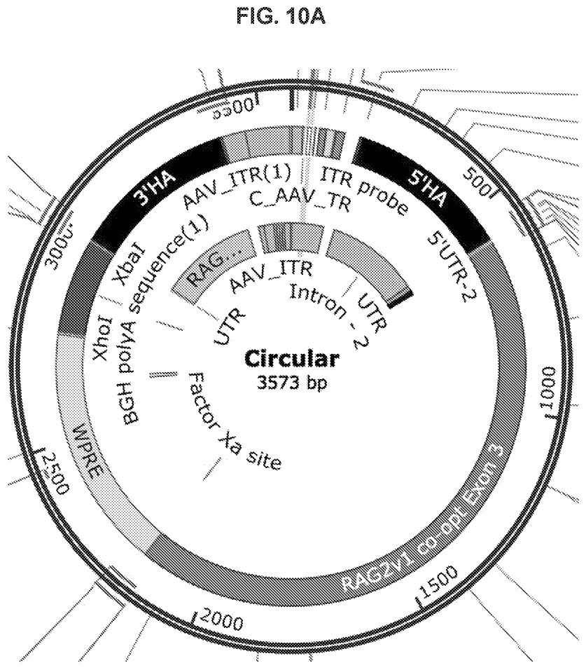

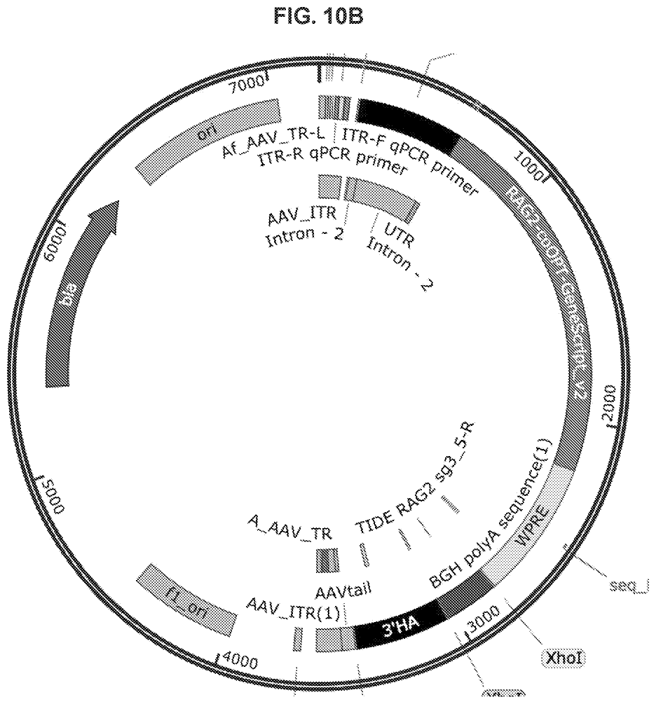

Finally, we compared the quality of in vitro T-cell differentiation of control-derived iPSCs in the ATO system and in the OP9-DLL1 monolayer system. Prior art has demonstrated similar differentiation results and kinetics using the DLL1 and DLL4 ligand [17]. As compared to the polyclonal pattern of rearrangements at both the TRB and TRA loci detected in the ATO system (FIG. 3A), healthy donor iPSCs differentiated upon co-culture with OP9-DLL1 cells demonstrated a polyclonal pattern of TRB rearrangements, but a restricted usage of TRA genes. In particular, iPSC derived T cells showed preferential usage of the most downstream TRAV and most upstream TRAJ genes (FIG. 9A), suggesting reduced viability of T cell progenitors in this system as compared to cells cultured in the ATO platform. These data support superiority of the ATO method for T-cell differentiation of iPSCs.

### DISCUSSION

RAG deficiency is a prominent cause of SCID. In a recent study, it was found to account for 19.2% of all cases of SCID identified in the United States and Canada in the period 2010-2018, representing the second most common genotype after IL2RG gene defects [22]. Moreover. RAG deficiency emerged as the most common form of atypical SCID (accounting for 29.6% of these cases), a condition characterized by residual and perturbed immune function, with clinical manifestations of immune dysregulation. Patients with RAG deficiency have a poor prognosis, unless immune reconstitution is achieved. In particular, severe forms of RAG deficiency are fatal early in life; among patients with hypomorphic mutations manifesting with combined immune deficiency with granulomas and/or autoimmunity (CID-G/AI), treatment refractory autoimmune cytopenias are very common, and a high mortality rate has been reported in childhood and young adulthood [23]. Allogeneic HSCT represents the mainstay of treatment for RAG deficiency; however, graft-versus-host disease, graft failure and incomplete immune reconstitution remain significant challenges. In a series of patients with RAG deficiency who received haploidentical HSCT at three major centers between 1985 and 2009, a very high rate (75%) of graft failure was observed among recipients of unconditioned HSCT, so that repeat HSC′T had to be frequently used, and none of the patients who engrafted in the absence of myeloablative conditioning reconstituted B-cell immunity [24]. Use of chemotherapy allowed improved immune reconstitution, but was associated with inferior survival, reflecting treatment-related toxicity [24]. Data from the Primary Immune Deficiency Treatment Consortium have indicated that improved outcome has been obtained in more recent years; however, they also confirmed a high rate of graft failure and poor T- and B-cell immune reconstitution in the absence of conditioning chemotherapy [25]. It has been speculated that impaired immune reconstitution after unconditioned HSCT in patients with RAG deficiency may reflect competition between autologous, genetically-defective lymphoid progenitor cells and donor-derived cells. This competition may extend up to relatively late stages of T cell differentiation, as suggested by our observation that bone marrow-derived CD34″ cells from RAG-deficient patients can differentiate into DP T cells in vitro when cultured in the ATO system [18], and confirmed here when differentiating patient-derived iPSCs.

Gene therapy represents an alternative approach to attain immune reconstitution in patients with SCID. When compared to allogeneic HSCT, edited autologous cell therapies have the advantages of overcoming difficulties in finding matched donors, as every patient provides their own donor cells, as well as mitigating the risk of GvHD and requisite immunosuppression, as autologous cells have exact HLA matches. Excellent results have been recently reported with gene therapy using self-inactivating lentiviral vectors in patients with X-linked SCID [26]. However, preclinical data in Rag-deficient animals have been less successful. In particular, variable efficiency of T and B cell reconstitution has been reported in Rag1^{-/-} mice treated with lentivirus-based gene therapy [7], and severe immune dysregulation with lymphocytic infiltrates in multiple organs and production of autoantibodies have been reported by another group [5]. Better results have been observed with the use of lentiviral vectors to correct the immunodeficiency in Rag2^{-/-} mice [27], though when a similar strategy was applied to a hypomorphic mouse model (Rag2^{R229Q}) the T and B cell count of reconstituted animals remained lower than normal [6].

Of major concern for the clinical application of lentivirus-based gene therapy for RAG deficiency is the observation that RAG gene expression is tightly regulated through the cell cycle and along lymphoid development. Dysregulated expression might increase the risk of leukemic transformation and autoimmunity. Moreover, conventional gene therapy (i.e., with gene addition, as opposed to gene correction) carries the additional risk that the endogenous mutant allele might interfere with the wildtype RAG cDNA introduced with the lentiviral vector.

New approaches are needed in order to expand the clinical toolkit available for treating RAG2 deficiency. Ex vivo CRISPR/Cas9-AAV6 mediated homology directed repair gene therapy approaches have been shown to be highly efficient and precise at correcting endogenous pathogenic mutation in healthy and patient-derived HSPCs [16, 28, 29]. Ex vivo gene targeting circumvents challenges presented by in vivo Cas9 editing, such as delivery of the gene editing machinery, specificity of targeted cells, preexisting adaptive immunity to spCas9 proteins, and precision of expression times for targeted nucleases which can compound the risk of generating off-target editing [30, 31].

This study shows that RAG2 is an exceptional target for this type of gene replacement therapy. Because the entire RAG2 amino acid sequence is encoded by a single exon, replacing this exon minimizes the risk of interfering with transcriptionally required intronic regulatory elements, while still correcting all reported patient RAG2 ORF mutations [32]. Additionally, a single copy of the corrected template DNA restores RAG2 function, as indicated by the observation that the parents of RAG2-deficient patients are heterozygous for the gene defect, and yet they do not manifest clinical or immunological abnormalities.

Taken together, this work shows that targeted on-site integration of a wild-type transgene allows by-passing of the developmental block due to RAG2 deficiency, robust RAG2 catalytic activity, and generation of a diverse TCR repertoire, comparable to that of an immunocompetent donor. The combination of these data show that this strategy effectively restores RAG function.

### Experimental Procedures

Generation and Characterization of iPSC Lines

Fibroblasts were cultured from a skin biopsy specimen obtained from the RAG2 deficient patient upon informed consent according to protocol 04-09-113R approved by Bostonn Children's Hospital IRB. Primary fibroblasts from patient and control foreskin fibroblasts (ATCC, Manassas, VA) were reprogrammed into iPSC using CytoTune-iSP 2.0 Sendai Reprogramming Kit (ThermoFisher) following kit instructions for feeder-dependent fibroblasts under IRB-approved protocol 16-I-N139, iPSC were analyzed by G-band karyotyping (Cell Line Genetics, Madison, WI) to ensure genomic integrity.

### Gene Editing

All single guide RNAs (sgRNAs) used in this study were purchased from Trilink Biotechnologies (San Diego, CA, USA) and were HPLC purified. All sgRNAs also contained three 2′-O-Methyl phosphorothioate modifications at the S′ and 3′ ends. The 20 bp sgRNA used for this work was of the sequence 3′-TGCAGAGACATAGTTTCTGA-5′.

Recombinant High Fidelity Cas9 was used in all editing experiments from Integrated DNA Technologies. All RNPs used for editing were created using a 1:3 Cas9:sgRNA ratio and were allowed to complex at room temperature for 30 minutes prior to electroporation. 1×10⁶ iPSCs were resuspended in 20 uL OPTI-MEM (Thermo Fisher) and then combined with the RNP and inserted into a single well of a nucleovette strip (Lonza). Cells were electroporated using a Lonza 4D Nucleofector (program CA137) and all 1×10⁶ cells were then recovered in one well of a 6-well plate coated with Vitronectin (Thermo Fisher) in Essential 8 Flex Medium (Thermo Fisher) supplemented with 10 μM ROCK Inhibitor (Tocris Bioscience, Bristol, UK). Plated cells were immediately transduced with rAAV6 at 50,000-250.000 vector genomes/cell as tittered by qPCR.

Quantification of Homologous Recombination by Digital Droplet PCR (ddPCR)

Analysis of the percentage of cells with successful integration of the RAG2 cassette was carried out by ddPCR using the National Institutes of Health Core Genomics Facility. Genomic DNA extracted from all target cells was purified using a DNEasy Blood and Tissue Kit (Qiagen*). 100 ng of purified gDNA was then combined with WT FAM Probe: 5′-CCCGAGGAACGTGACCATGGAGTGGC-3′ along with forward primer: 5′ GCACAGGAAGTTTAGCAGTG-3′ and reverse primer: 5 GGGAATTCAAGACGCTCAGA-3′ and MUT HEX Probe: 5′-GAGCCTGCAGATGGTGACCGTGTCCA-3′ along with forward primer: 5′-GCACCTTCGGCTAGTCTTTA-3′ and reverse primer: 5-ATCAGAGAAAAGCCTGGCTG-3′ at a primer/probe ratio of 900 nM/250 nM and in a total reaction volume of 22 μL including 11 uL ddPCR Supermix for Probes (No dUTP) (BioRad). Droplet generation and PCR/reading was performed by the Genomics Core with cycling parameters of: 95C (10 minutes), [94C (30 seconds), 61.7C (30 seconds), 72C (2 minutes)-repeated steps in brackets 50 times], 98C (10 minutes), −4C (indefinite) on a QX200 Droplet Digital PCR System (Bio-Rad).

### Analysis of INDEL Frequency and Identity

Freshly purified CB-derived CD34⁺ HSPCs, were obtained through the Binns Program for Cord Blood Research at Stanford University, under informed consent. INDEL frequencies and identities were examined using the ICE tool from Synthego. Genomic DNA from cells exposed to the target RNP was isolated 2 days post electroporation and the PCR product was then cleaned up using a Qiagen PCR Cleanup Kit* before being submitted for Sanger sequencing using the following primers (also used for PCR): forward: S′-ATGTGGTTCTTTCAGCTGACG-3′ and reverse: 5′-CGAAAAGTAACCTTTTTGTTGT-3′ with an appropriate mock control genomic DNA sample used as the baseline for deconvolution.

### Analysis of Off-Target CRISPR Cas9 Effects by Whole Genome Sequencing

Whole genome sequencing of the precursor and edited cell lines was performed to a median depth of 30× using the Illumina NovaSeq 6000 system. Reads were trimmed using Trimmomatic v0.39 and mapped using BWA-MEM v07.17 to the human hg38 reference genome with the inserted construct sequence added as an additional contig [33]. PCR duplicates were marked using Samblaster v0.1.2.5 (broadinstitute.github.io/picard/), and GATK v4.1.9.0 was used to perform base recalibration [34]. The genomic insertion location was validated by examining split reads from the edited sample, and identifying the location of reads with one pair member mapped to the construct and the other pair member mapped to the human genome.

To identify potential off-target mutations, SNPs and INDELs were called in a paired fashion using MuTect2 from GATK v4.1.9.0 and following the GATK Best Practices (gatk.broadinstitute.org/hc/en-us/articles/360035531132—How-to-Call-somatic-mutations-using-GATK4-Mutect2) [35]. To reduce false-positive calls, variants were filtered with the following criteria: edited_sample_depth 15, edited_sample_alt_count >5, edited_sample_freq >0.1, precursor_sample_depth >15, precursor_sample_alt_count=0.

### Isolation and Culture of MS5-hDLL4 Cells

The murine stromal cell line (MS5) edited to ectopically express human Notch ligand, delta-like 4 (hDLL4) was used as the hEMP co-culture cell type for the ATOs. MS5-hDLL4 cells were cultured in Dulbecco's Modified Essential Medium (DMEM) supplemented with 10% fetal bovine serum (FBS) (Thermo Fisher Scientific, Waltham. MA, USA) and 100 μg/mL Primocin (InvivoGen, San Diego, CA, USA). For ATO seeding, MS5-hDLL4 cells were used at a confluency of approximately 70% and were dissociated with TrypIE Express.

Generation and Isolation of Human Embryonic Mesodermal Progenitors (hEMPs)

Mesodermal progenitors were induced from iPSCs as described with certain optimizations. Briefly, iPSCs were cultured on Vitronectin coated played in Essential 8 complete medium. At the time of induction, cells are 60-70% confluent and are dissociated from the well using Tryp1E at 37 ºC for 10 minutes. Disassociated cells are then washed and resuspended in X-Vivo 15 medium (Lonza) supplemented with rhActivin A (R&D Systems), rhBMP4 (R&D Systems), rhVEGF (R&D Systems), rhFGF (R&D Systems) at 10 ng/ml and ROCK Inhibitor at 10 μM (Tocris). After counting, 2.5×10⁶ cells are seeded into a single Vitronectin coated well in a 6-well dish in 2 mL X-Vivo 15 media supplemented with the same factors. Medium was then changed each day with the same growth factors as above without ROCK inhibitor. On day 5, cells are incubated with 1× Accutase (Stem Cell. Vancouver, Canada) for 10 minutes at 37° C. and washed twice with PBS before staining with EPCAM and CD56 antibodies to prepare for fluorescence activated cell sorting. Stained cells were sorted on a FACS ARIA instrument (BD Biosciences, San Jose, CA) for CD56⁺ EPCAM-cells.

### T Cell Differentiation

ATOs were generated as described with some minor alterations. Briefly, 10.000 EPCAM-CD56⁺ hEMP cells were combined with 0.5×10⁶ MS5-hDLL4 washed and counted cells per ATO resuspended directly in EGM2 (Lonza) media with 10 μM TGF-βR1 inhibitor SB-431542 (SB Blocker) (Toeris Bioscience) and ROCK Inhibitor. Enough cells were combined in a 1.5 mL Eppendorf tube to accommodate 6 ATOs. Combined cells in 1.5 mL Eppendorf tubes were centrifuged at 1400 RPM for 5 minutes at 23° ° C. in a swing bucket centrifuge to pool cell aggregates to the bottom of the tube. The media was aspirated out and enough complete media was added to the cell slurry such that the total volume was 36 μL (6 μL per ATO with 6 ATOs in each tube). Two 6 μL droplets of the resuspended cell slurry were then placed with a p20 pipette onto a pre-wet Millicell Transwell Insert (EMD Millipore, Billerica, MA) Membrane sitting in 1 mL of complete EGM2 media in a single well of a six well plate. The media was then changed every other day. At day 7, the media was further supplemented with hematopoietic cytokines rhTPO (Peprotech), rhFLT3L (Peprotech) both at 5 ng/ml, and rhSCF (Peprotech) at 50 ng/mL. At day 14 the media is changed to “RB27” which consists of RPMI 1640 (Gibco, Waltham, MA, USA), 4% B27 (ThermoFisher Scientific. Grand Island. NY), 30 UM L-Ascorbic Acid (Sigma-Aldrich. St. Louis, MO) resuspended in 1×PBS, 1% Glutamax (ThermoFisher Scientific, Grand Island, NY), 100 μg/mL Primocin, rhIL7 and rhFLT3L at 5 ng/mL as well as SCF at 10 ng/ml. Media was changed twice weekly. From weeks 3 to 5 of RB27 culture, cells were harvested from ATOs by adding 1 mL MACS buffer (PBS with 5% BSA and 0.5M EDTA) to each well, physically disrupting the ATOs using two p1000 tips, and then further pipetting to dissociate the ATO fragments from the membrane. Cells were then centrifuged and resuspended and counted in FACS buffer before being stained with antibodies on a BD LSR II Fortessa (BD Biosciences, San Jose, CA) and analyzed using FlowJo software version 10.5.3 (FlowJo, Ashland, OR, USA).

### TCR Sequencing and Data Visualization

TCR sequencing was conducted by Adaptive Biotechnology (Seattle, WA, USA) using their immunoSEQ assay service and analyzer. Approximately 1 μg of genomic DNA from CD3⁺ sorted ATO derived T cells was submitted to their facilities for sequencing. Sample data was generated using a two-step amplification bias-controlled multiplex PCR strategy to amplify each V and J gene. Amplicons were then amplified again to adapt barcodes for Illumina next generation sequencing. Raw demultiplexed data was downloaded and analyzed using R to generate heatmaps. Briefly, recombination events between genes at the TRA′D and TRB loci were imported separately into the R programming environment. Once imported, the number of recombination events observed between each pair of genes was transformed into a percentage of total recombination events observed. Genomic loci for genes was downloaded from NCBI and genes were ordered by their chromosomal position. To aid in visualization the percentage of recombination events per gene pair was scaled by square root transformation. Software program Treemap version 2019.4.2 was used to generate treemap illustrations of CDR3 identities and frequencies within the sequenced population. The Shannon's Entropy Index score was tabulated using software program Past4.03.

### Statistical Tests

Statistical tests used in the data shown in this paper were generated using Graph Pad Prism8 Software version 8.4.1. Details of each test are included in the figure description.

### Example 2. A Genomic Editing-Based Therapeutic Approach for RAG2 Deficiency

Recombination-activating gene 2 (RAG2) deficiency is classified as a severe combined immunodeficiency disorder (SCID), where the adaptive immune cells are unable to properly assemble functional antigen-specific receptors of the immunoglobulin (BCR) and T-cell (TCR). RAG2 functions as part of the complex required for V(D)J-recombination activity, which is essential during lymphocytes development and antigen-recognition. Less than 1% V(D)J-recombination activity leads to typical RAG2-SCID, with a complete absence of circulating T and B cells (T−B−NK+), where >1% V(D)J-activity leads to atypical RAG2-SCID classified as Omenn syndrome (OS), expansion of γδ-T cells and, in some patients, granulomatous inflammation and/or autoimmunity. Hematopoietic stem cell transplantation is a curative therapy for patients with typical RAG2-SCID and OS. However, in the absence of an HLA-matched donor, high incidence of graft failure and poor immune reconstitution limits the therapeutic success. Although conventional approaches to gene therapy are being considered for RAG2 deficiency, they carry theoretical risks of genomic instability associated with dysregulated expression of the gene. To address these issues, we report a CRISPR/Cas9 based proof-of-concept genome editing approach designed to correct all pathogenic mutations in the RAG2 gene.

Our approach uses adeno-associated viral vector of serotype 6 (AAV-6) to deliver a codon-optimized RAG2 (RAG2co) therapeutic transgene at the endogenous RAG2 gene translation initiation site in human hematopoietic stem and progenitor cells (HSPCs). RAGco bulk allele genome targeting (GT) analysis shows a median 40.5% (range 21.0%-53.3% n=7), while single cell analysis confirms 71% alleles targeting efficiency (30/42 clones) with 30.9% mono-allelic and 40.5% bi-allelic GT.

RAG2co GT into healthy donor-derived HSPCs engrafted (median=15.8%, bone marrow, BM) into immunodeficient NSG mice (n=11) at no statistical difference from control cells (n=5 mice per condition). 18 weeks post engraftment analysis of sorted human cells derived from RAG2co GT HSPCs, showed a median bulk allele GT level of 51.0% (range 16.6%-70%, n=4 mice) and 34.6% (range 12.3%-64.0%, n=7 mice) in BM and spleen of mice, respectively. Human sorted T-cells (n=4 mice) derived from BM of mice engrafted with RAG2co GT HSPCs showed a median of 52.3% (range 40.0%-70.0%) bulk allele GT, while spleen-derived and sorted T-cells and B-cells bad a median of 44.5% (range 16.6%-64.0%, n=4 mice) and 25% (range 12.3%-46.5%, n=3 mice), respectively. Engraftment analysis of the RAG2co GT HSPCs confirmed multi-lineage reconstitution.

Lastly, we report 40% bulk allele GT in one RAG2 patient (c.296>A: c1242C>A) frozen mobilized CD34 HSPCs. In vivo studies are currently underway to assess the engraftment potential and functional rescue of patient-derived RAG2 HSPCs. Off-target analysis by next generation sequencing (NGS) of RAG2 patient GT HSPCs (RNP condition), identified insertion and deletions (INDELs) in only 2 out of the 48 COSMID predicted sites, at levels <0.2% located at an intergenic (>38 kb from nearby gene) and intronic (MIR-383) loci.

Our preclinical data, thus far, demonstrates a robust, precise and efficient next generation gene therapy treatment for RAG2 deficiency.

### Example 3. RAG2 Gene Editing in HSPCs

Xenotransplantation studies were carried out using human hematopoietic stem cells from healthy donors. Cells were cultured for four days and their genomes targeted with RAG2 cDNA, and they were then injected intrahepatically into 2-3 day old immunodeficient mice. End point analysis was then performed after 20 weeks from bone marrow, spleen, and peripheral blood. T and B cells were sorted from bone marrow and spleen of the mice, and the frequency of RAG2 genome targeting was quantified by digital droplet PCR. We observed median frequencies of 52% and 45% among T cells in the bone marrow and spleen, respectively, and of 25% among B cells from the spleen.

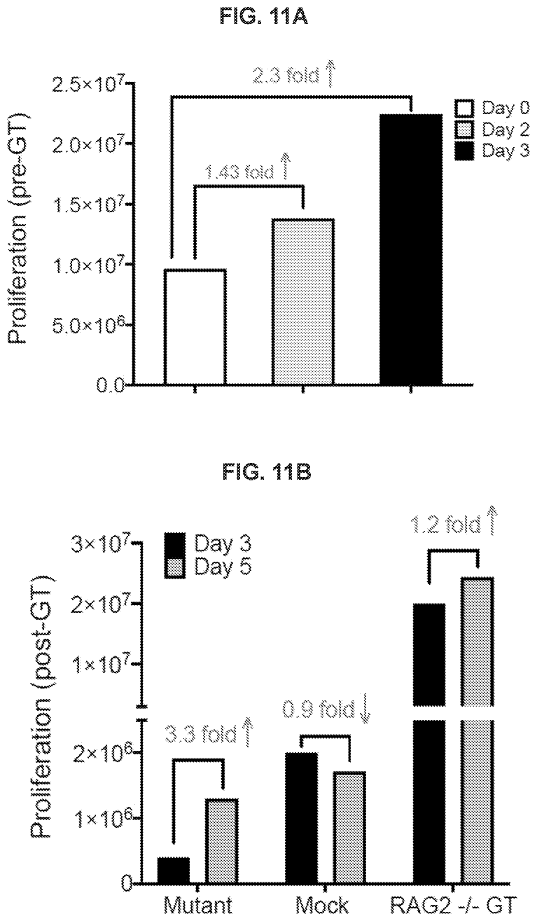

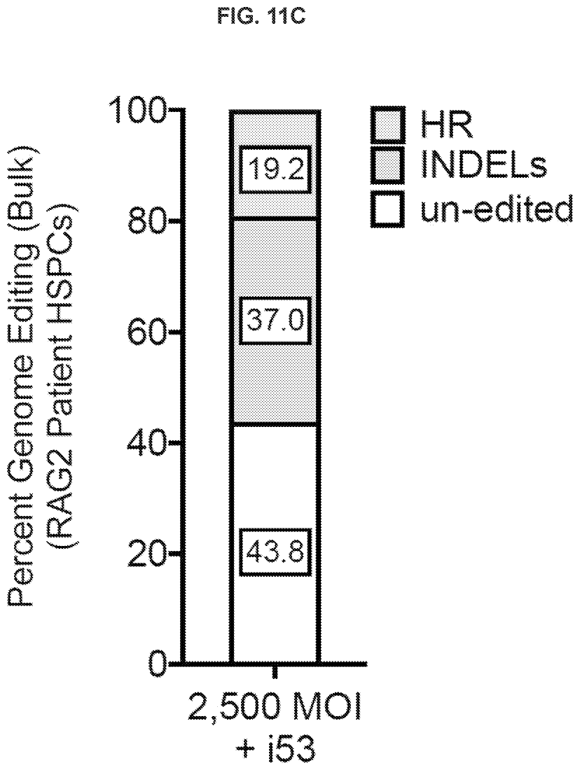

Genome targeting was performed in HSPCs from SCID-RAG2 patients. Targeting the RAG2 locus allowed a 3.3-fold increase in the proliferation of the mutant cells (FIG. 11B). The outcome of genome targeting of RAG2 patient HSPCs using a RAG2 cDNA donor template, delivered by rAAV at 2,500 MOI in the presence of recombinant i$3 protein, is shown in FIG. 11C.

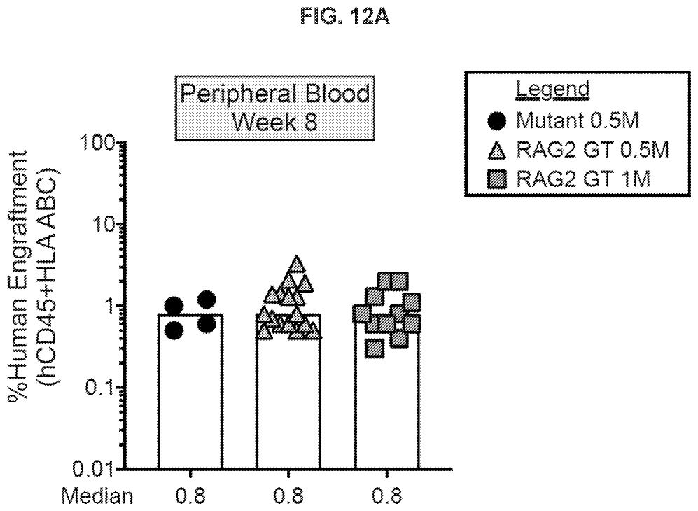

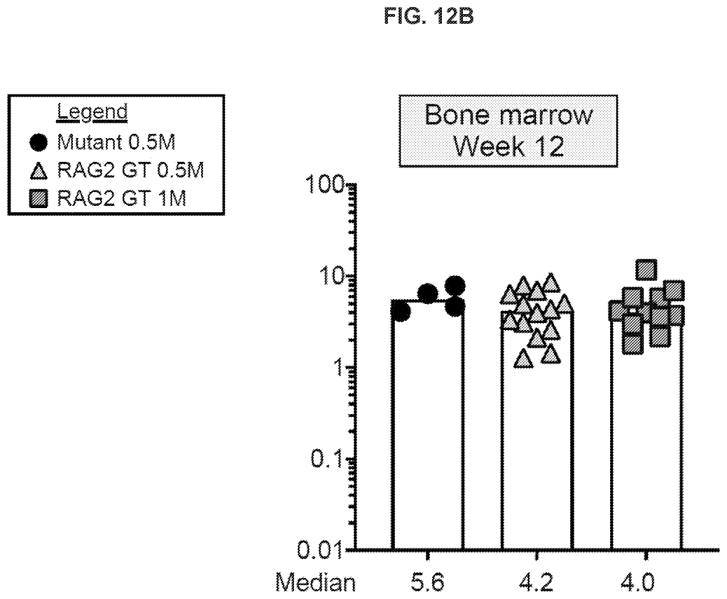

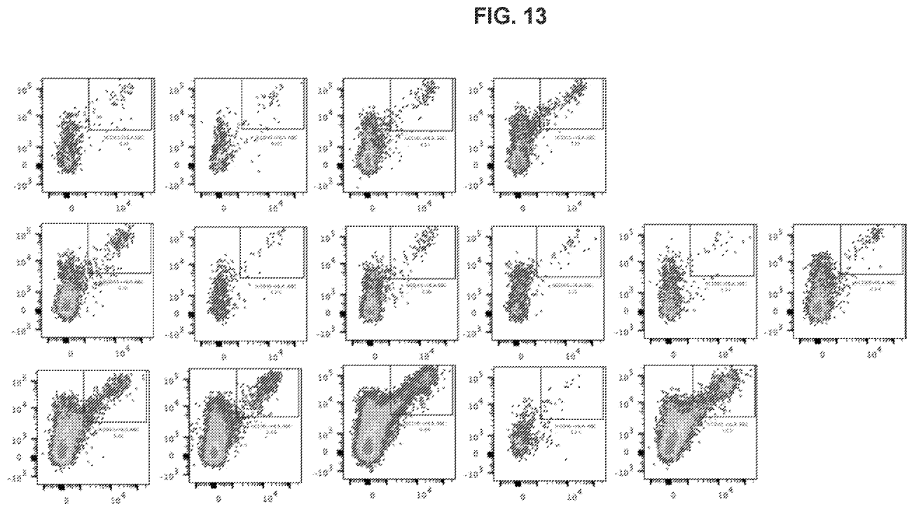

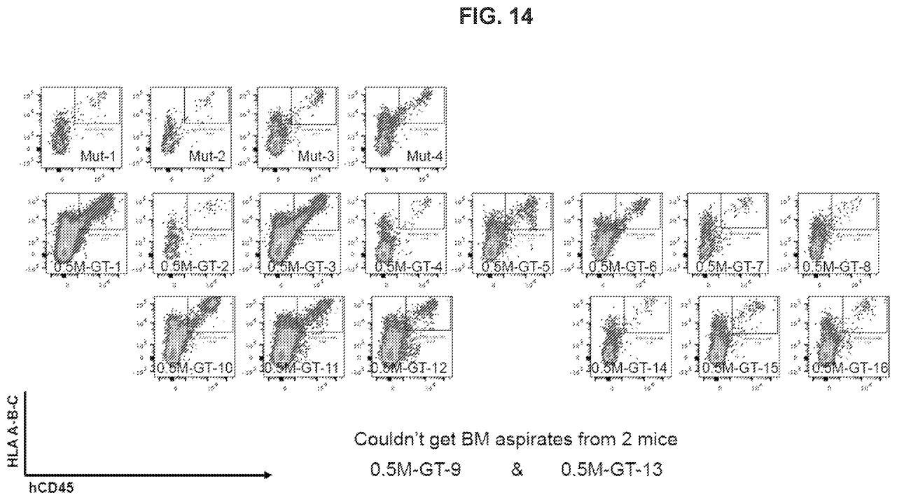

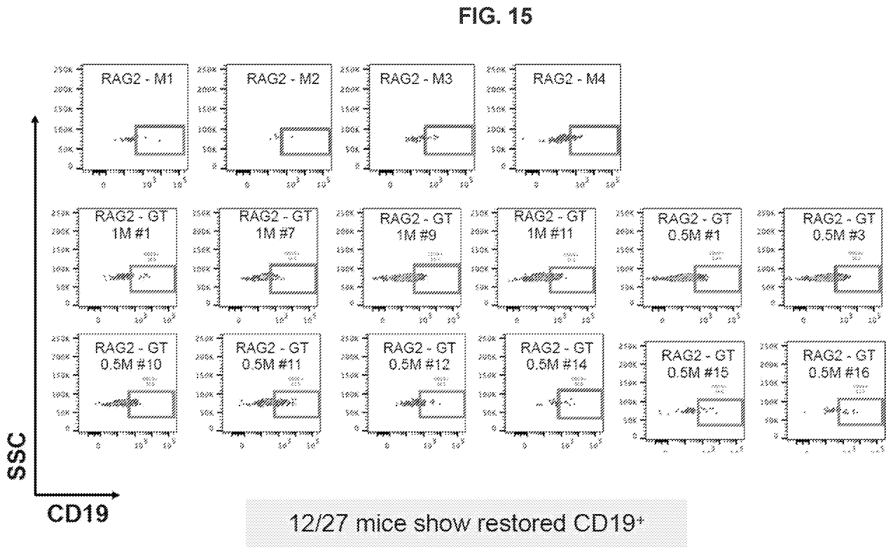

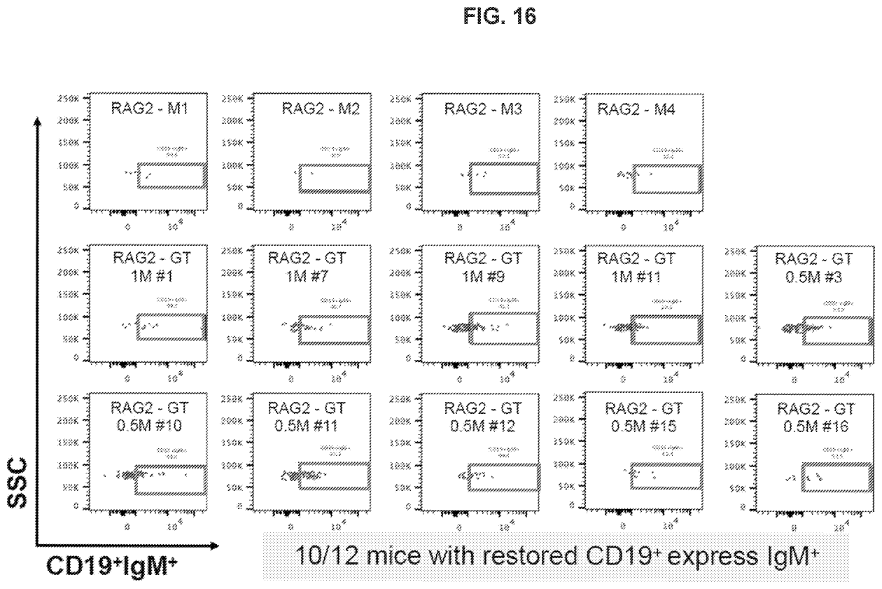

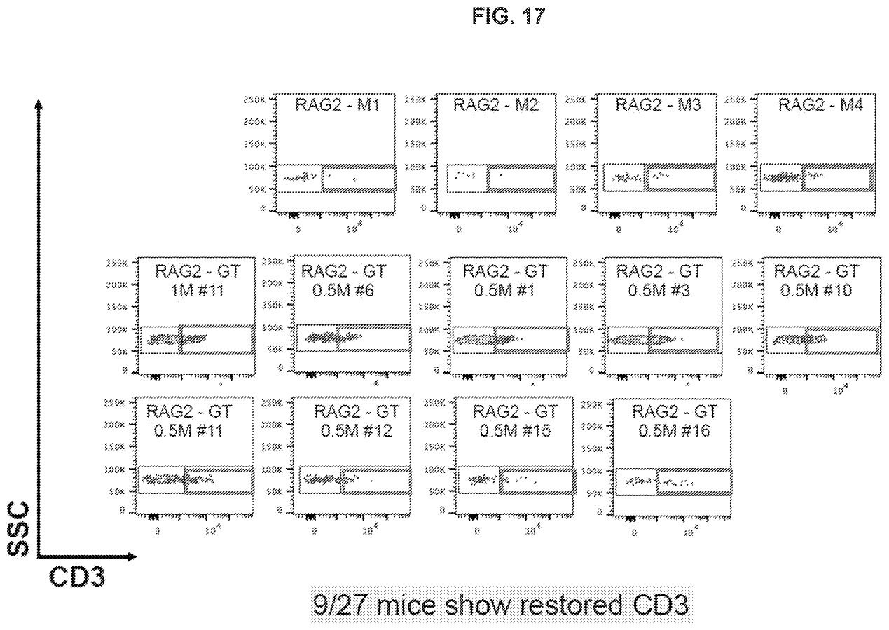

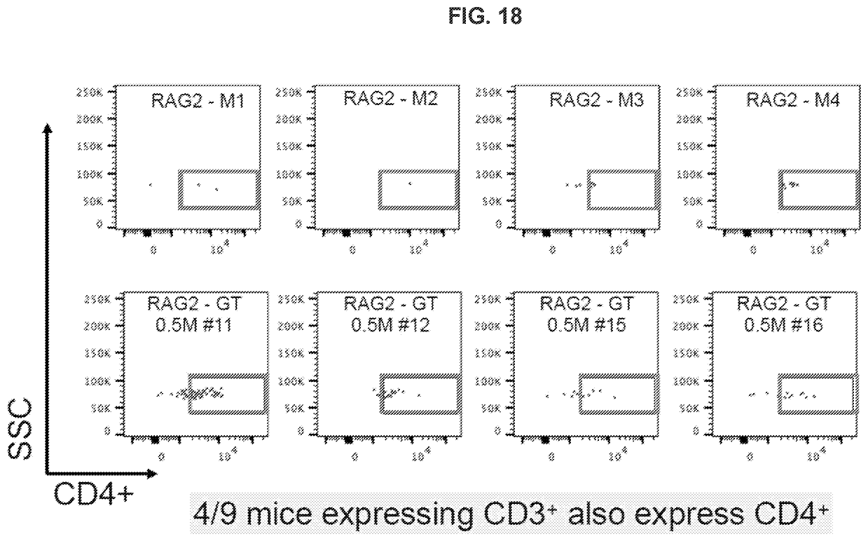

Genome targeted SCID-RAG2 HSPCs were transplanted, and the percent engraftment was determined at week 8 in the peripheral blood (FIG. 12A) and bone marrow at week 12 (FIG. 12B). FACS plots were generated to show bone marrow engraftment with uncorrected and with 1 million (FIG. 13) or 500,000 (FIG. 14) corrected HSPCs. FACS plots generated to quantify B and T cells in bone demonstrated that 12 of 27 mice showed restored CD19⁺ B cells (FIG. 15), 10 of the 12 mice with restored CD19⁺ B cells also expressed IgM⁺ (FIG. 16), 9 of 27 mice showed restored CD3⁺ T cells (FIG. 17), and 4 of the 9 mice with restored CD3⁺ T cells also expressed CD4″(FIG. 18).

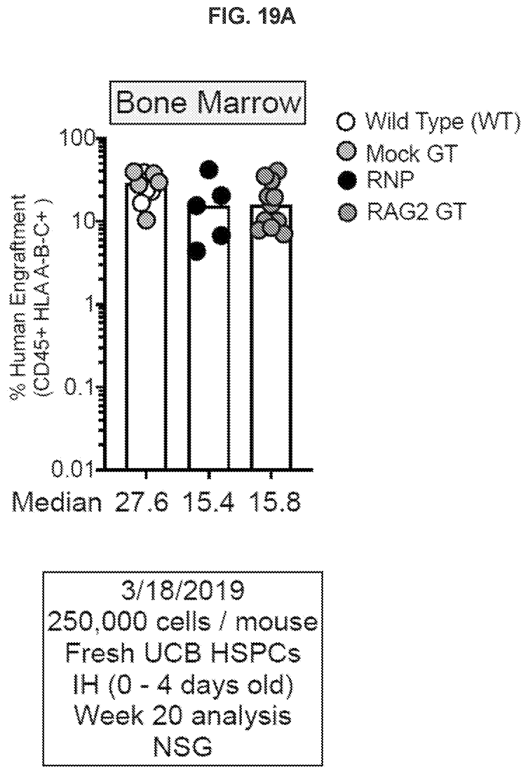

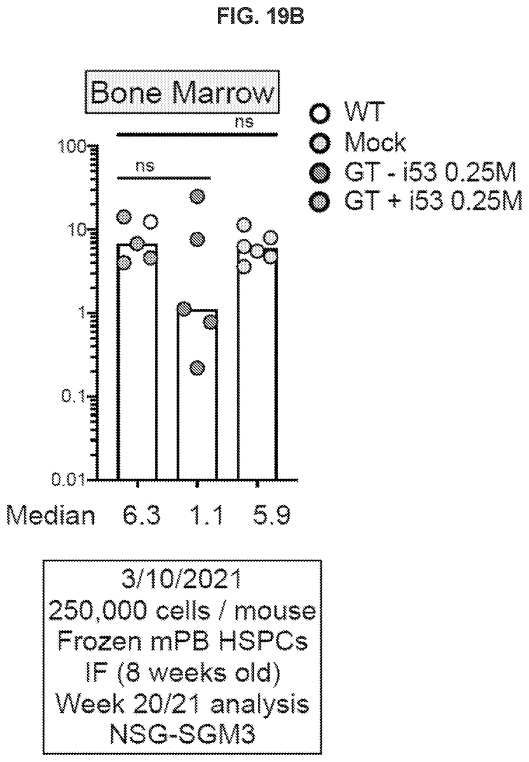

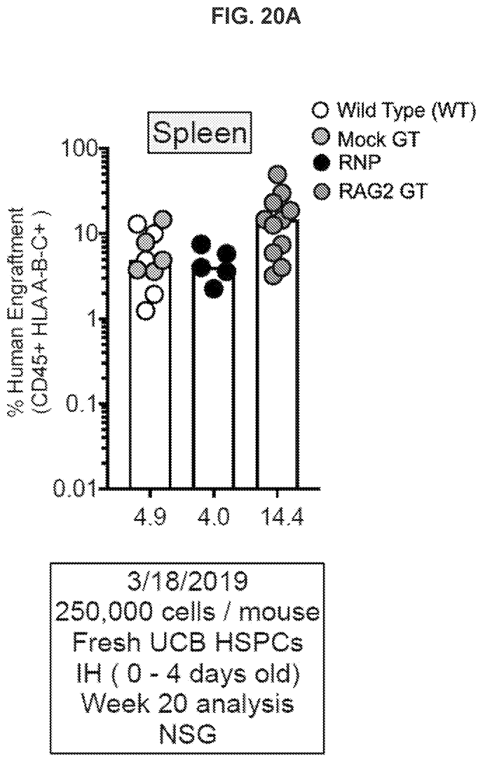

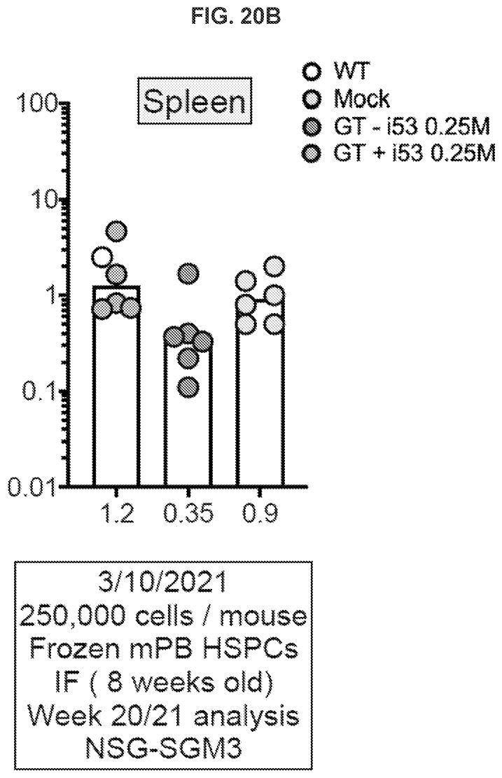

250,000 HSPCs from umbilical cord or peripheral blood were transplanted into NSG-SGM3 mice in the presence or absence of i53 protein, and their bone marrow (FIGS. 19A-19B) and spleen (FIGS. 20A-20B) analyzed at 20 weeks by FACS.

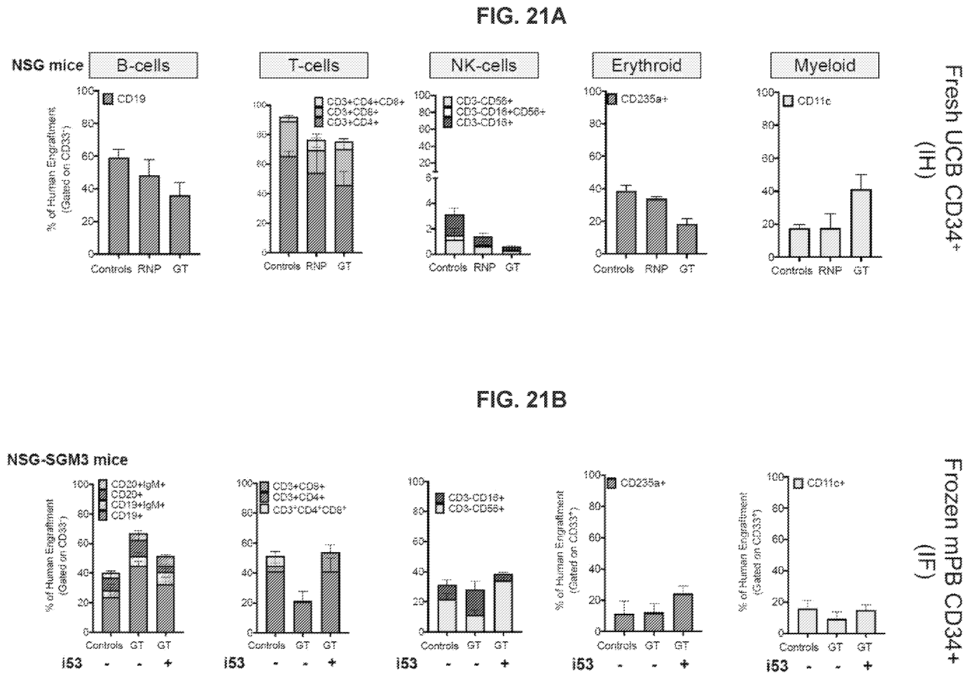

Healthy donor-derived HSPCs from umbilical cord (FIG. 21A) or peripheral blood (FIG. 21B) were gene targeted at the RAG2 locus and transplanted into mice. Multilineage analysis was performed at 20 weeks.

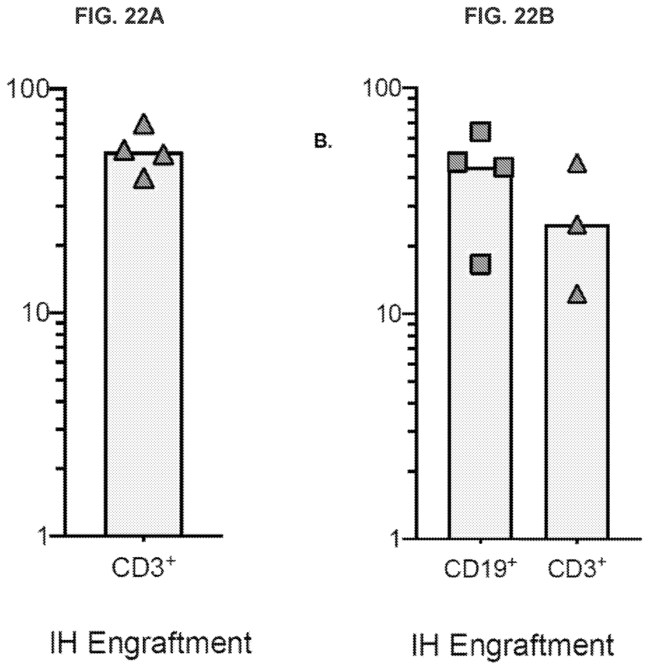

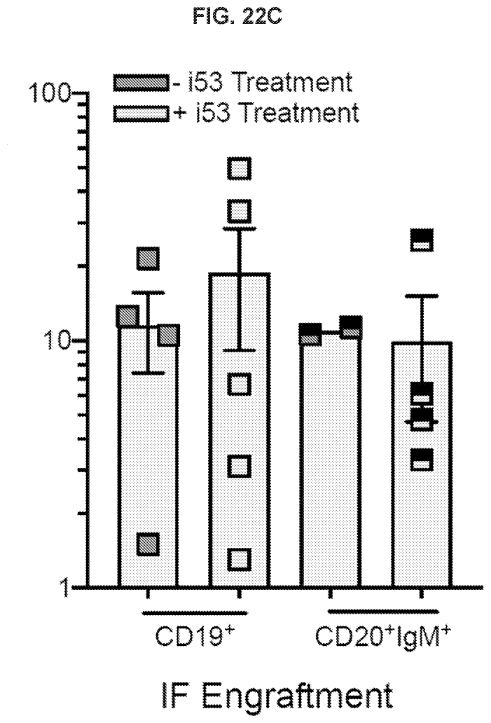

Human T cells (CD3⁺) were sorted from bone marrow (FIG. 22A), and human T and B cells (CD19′ or CD20′IgM⁺) were sorted from the spleen at 20 weeks post-transplant with cord blood (FIG. 22B) or peripheral blood (FIG. 22C), and the presence of RAG2 cDNA quantified by ddPCR.

### Example 4. Genetically Corrected RAG2-SCID Human Hematopoietic Stem Cells Restore V(D)J-Recombinase and Rescue Lymphoid Deficiency

Severe Combined Immunodeficiency (SCID) comprise a small and rare group of genetic diseases caused by inherited defects in genes required for T-, B-cell, and occasionally NK development and function [37]. Recombination Activating Gene 2 (RAG2)-SCID is the second most prevalent SCID. Loss of function (LOF) mutations in the RAG2 gene completely disrupt T- and B-lymphocytes' ability to establish functional cell surface receptors necessary for receiving proper developmental signals and mature [38]. RAG2 is part of a multimeric protein complex that ensures an extensive repertoire of T-cell and immunoglobulin receptors (TCR and BCR) is generated in the developing lymphocytes through a combinatorial association of the variable (V), diversity (D), and junction (J) gene fragments, a process referred to as V(D)J recombination [1, 39]. LOF RAG2 mutations reduce the V(D)J recombination activity to <1% resulting in a typical RAG2-SCID phenotype (TB-NK″) [40].

At variance with other SCID genes characterized by immune deficiency, RAG1 2 mutations cause additional immune dysregulation, translating into a spectrum of clinical manifestations [41, 42]. Specifically, severely hypomorphic RAG2 mutations permit residual protein activity, resulting in Omenn Syndrome (OS) with >1% V(D)J recombination activity resulting in oligoclonal T-cells [43, 44]. Unlike typical RAG2-SCID patients who only develop life-threatening infections early in infancy. OS patients have the extra burden of developing autologous autoreactive T cells that infiltrate the skin, gut, liver causing erythroderma, lymphadenopathy, hepatosplenomegaly, eosinophilia, in addition to severe hypogammaglobulinemia, with increased IgE levels. Hypomorphic RAG2 mutations can also cause an atypical form (AS) of RAG2-SCID in which patients have reduced numbers of T and B-cells and a decreased ratio of naïve T-cells [45]. Like typical RAG2-SCID, AS patients are highly susceptible to severe and opportunistic infections with the additional presentations of autoimmune manifestations [46], such as cytopenia or autoimmune hemolytic anemia caused by CMV-dependent expansion of TCRγδ⁺ T cells [45]. RAG2 mutations with residual protein catalytic activity can lead to delayed-onset of granulomas [47, 48] in combination with autoimmune manifestations (CID-G/AI) [49]. Though T- and B-cells are produced, CID-G/AI patients experience recurrent viral infections due to herpesviruses and human papillomavirus infections.

Current interventions for severe forms of RAG2 deficiency, including RAG2-SCID. OS, and AS require allogeneic hematopoietic stem cell transplantations (allo-HSCT) in the first year of life to establish immune reconstitution. Allo-HSCT achieves >80% survival rates when HLA-identical donors are available [24]. However, due to the uncertain availability of immunologically matched donors, patients often rely on haploidentical-HSCT, resulting in high rates (>75%) of graft failure and poor lymphoid reconstitution in the absence of myeloablative conditioning in patients that achieved sustained engraftment [24, 25]. In humans with RAG deficiency, genetically defective common lymphoid progenitors (CLPs) occupy the bone marrow and thymus niches [50]. In the absence of myeloablative chemotherapy, the donor HSPCs-derived early lymphoid progenitors cannot access the niche space necessary to establish a robust lymphoid immune reconstitution. Even when chemotherapy is administered, other factors are required to assure a successful transplantation outcome, including young age (<3.5 months of life), absence of infections at the time of transplant, and the diagnosis of a typical RAG-SCID genotype/phenotype [25, 51]. RAG^{-/-} patients that present with AS or OS form have a worse survival outcome than the typical RAG-SCID (T⁻B⁻NK⁺) [51].

Animal studies in RAG deficiency have shown variable therapeutic efficacy of HSPCs transduced with lentivirus particles carrying functional copies of RAG1 [5, 7] or RAG2 genes. For the RAG1 gene, the lymphoid lineage was only partially rescued by lentivirus addition of RAG1 cDNA, with some mice showing signs of inflammations resembling OS [5]. The therapeutic efficacy of LV-based delivery of a codon-optimized RAG2 (coRAG2) cDNA was more substantial but not complete [27]. B-cell immunity was only partially restored to levels sufficient to prevent immune dysregulation. Environmental factors have been implicated as triggers of immune tolerance dysregulation, especially when immune reconstitution was incomplete. Based on these, autologous transplantation of lentivirus modified HSPCs overexpressing RAG genes might not robustly and faithfully recapitulate the levels of regulation and expression necessary to support healthy immune system development and function, thus presenting concerns for use in clinical applications.

Advances in genome editing technology [52-54] have allowed in situ precise gene correction in human HSPCs. This process can offer significant therapeutic benefits for diseases where the underlying gene relies on strict spatiotemporal gene regulation and expression [55]. Cluster Regulatory Interspaced Short Palindromic Repeats-associated Cas9 nuclease (CRISPR/Cas9) is a genome engineering platform that has been successfully applied to primary human cells [15, 56, 57], including HSPCs [16, 57-60]. The system uses a 2′-O-methyl 3′phosphorothioate chemically modified guide RNA (sgRNA) to direct a high fidelity Cas9 nuclease to a pre-defined genomic site where it creates double-strand breaks and activates the endogenous non-homologous direct repair (NHEJ) and homologous direct repair (HDR) pathways. In the presence of adeno associated virus 6 (AAV6) that delivers a corrective donor DNA template carrying the desired genomic modification and flanked by homology arms to the break sites, the HDR pathway integrates the new DNA sequence through a homologous recombination-mediated genome targeting (HR-GT) process [55]. To maximize the therapeutic outcome by autologous transplantation of RAG2 genetically modified HSPCs, the functional protein must be expressed at endogenous levels in a cell cycle and lineage-specific manner, which can be achieved by inserting a functional codon-optimized RAG2 cDNA at the endogenous RAG2 transcription start site. This would establish a novel therapeutic strategy that would be independent of patient-specific mutations and compared to the lentivirus approach, would ensure a safer and predictable transgene expression.

In our previous publication, we used induced pluripotent stem cells (iPSCs) combined with the artificial thymic organoid (ATO) system, which is supportive of in vitro T-cell differentiation, and the CRISPR/Cas9-AAV6 tools, to replace a patient's RAG2 null mutation (c.831T>A) with functional coRAG2 cDNA [61]. We showed that this corrected the T-lymphocyte developmental blockage with progression through the double-positive (DP) stage and generation of CD3⁺ TCRαβ⁺ in RAG2 corrected cells, at comparable levels to CD3⁺ TCRαβ⁺ cells derived from healthy donor iPSCs. Furthermore, the V(D)J recombination impairment was restored in the RAG2. HR-GT cells. Similar numbers of cells were obtained in the healthy and RAG2^{-/-} HR-GT corrected groups. Our analysis of the T-cell receptor diversity at the T-cell receptor beta (TRB) and T-cell receptors alpha/delta (TRA TRD) loci confirmed a polyclonal pattern of rearrangement in receptors of CD3⁺ T-cells derived from RAG2-HR-GT. Together, our data demonstrated robust correction of the RAG2-specific defect in vitro in the T-lymphoid lineage. However, the in vivo efficacy of the approach remains to be determined.

Here, we develop a “universal” genome targeting strategy for correcting RAG2 deficiency based on on-target integration and show complete reconstitution of the lymphoid lineage development from healthy human donors and RAG2^{-/-} deficient patient-derived HSPCs. We describe the in vivo correction of HSPCs carrying a heterozygous compound RAG2 mutation that completely abolishes the lymphoid lineage development. We report that the patient-derived genome corrected HSPCs that express coRAG2 transgene from the RAG2 locus, sustained long-term engraftment, restored V(D)J activity, lymphoid developmental block, and corrected NK cells immature phenotype in an immunodeficient NSG-SGM3 mouse model. Engrafted mice with RAG2^{-/-} HR-GT HSPCs showed no signs of inflammation or tumors and survived for a total of 20 weeks.

### Results

### Efficient “Universal” Genome Targeted Strategy of RAG2 to the Endogenous Locus in Human HSPCs

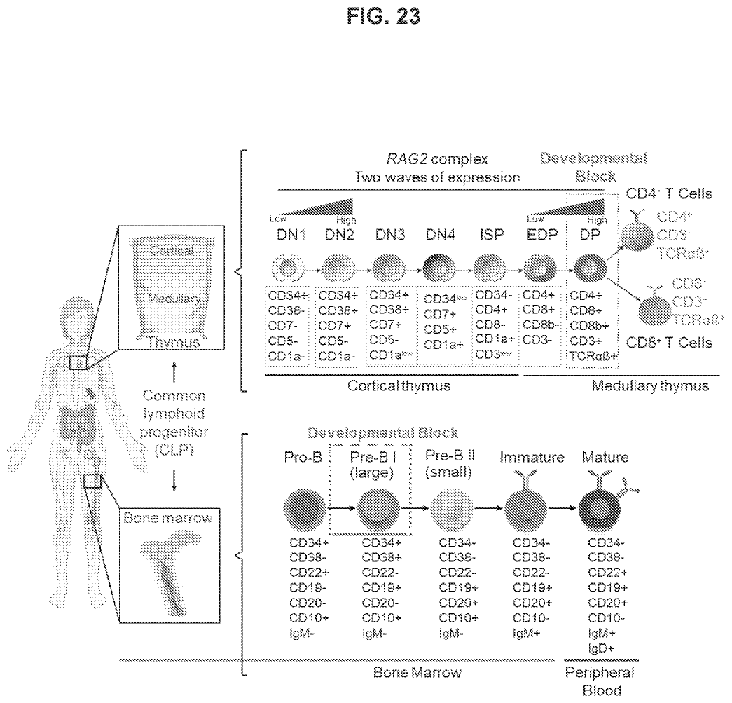

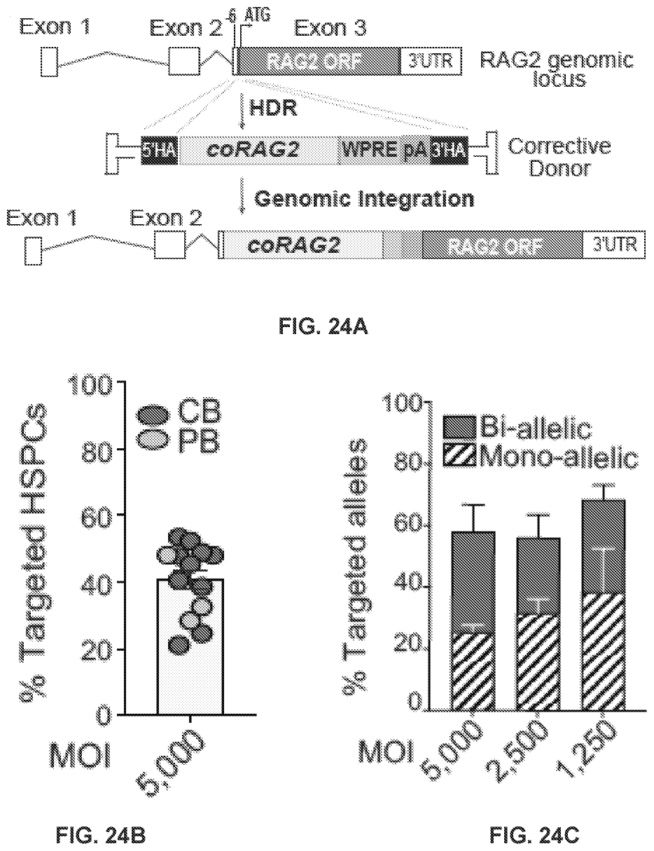

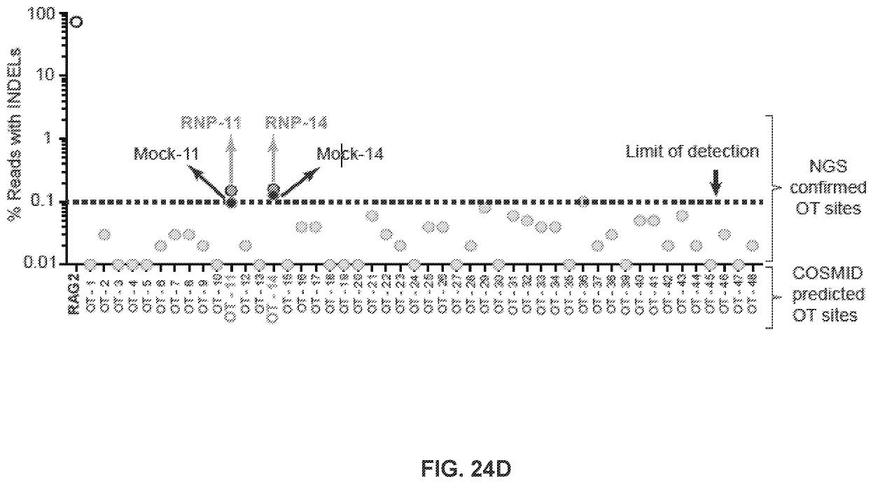

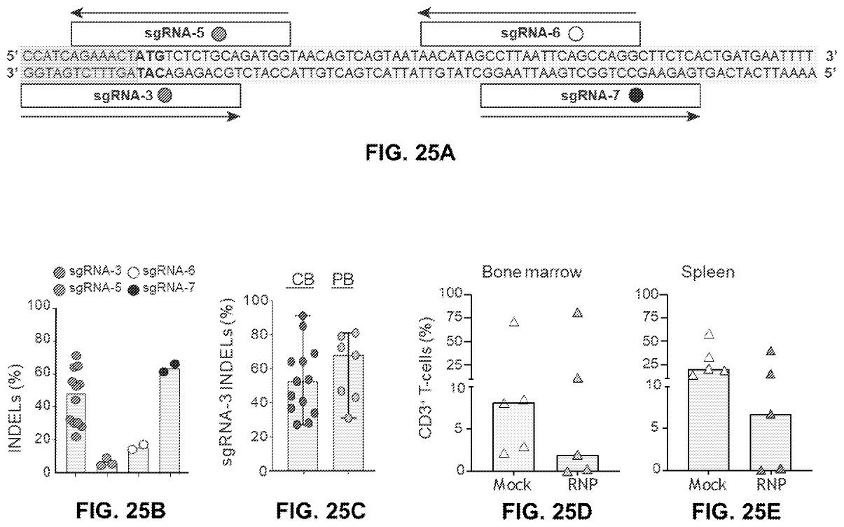

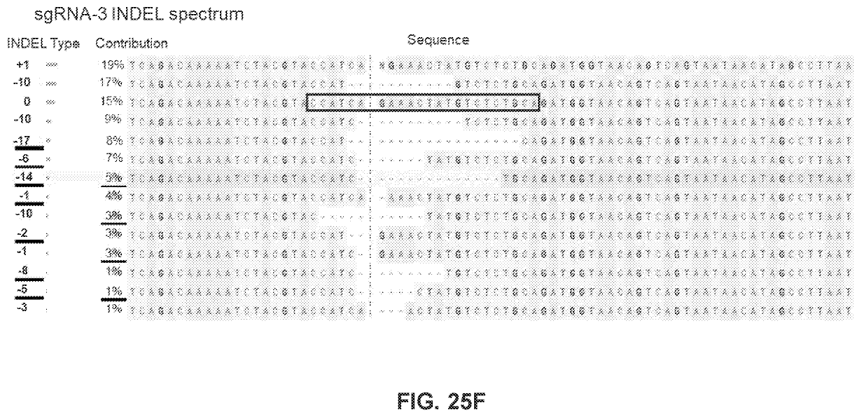

Two central concepts of our corrective therapeutic approach for RAG2 deficiency are that HSPCs and their progeny will preserve the physiological gene regulation necessary to achieve temporal and lineage-specific RAG2 protein expression and activity (FIG. 23) and that the vast majority of RAG2-causing mutations, including deletions, will be corrected. Using a carefully screened sgRNA complexed with high fidelity (HiFi) S.p. Cas9 ribonucleoprotein (RNP), we designed a genome targeting strategy aimed at integrating a codon-optimized RAG2 cDNA (coRAG2) at the endogenous transcription start site (FIG. 24A). The sgRNA selected for our strategy (sgRNA-3, FIGS. 25A and 25B) cuts six base pairs upstream of the ATG transcription start site, correcting all pathogenic mutations located at the start site and downstream, following genomic integration of the transgene. While the codon optimization of the RAG2 open reading frame disrupts the sgRNA binding site, the unmodified base pairs present in the 5′ untranslated region, including the protospacer adjacent motif (PAM) sequence, remain intact. To assure that the integrated donor will not be re-cleaved, we introduced two mutations in the PAM sequence (CCA>GGA) located in the 5″ homology arms of the donor. The RNP complex was electroporated into fresh cord blood-derived (CB) and frozen adult peripheral blood-derived (PB) HSPCs. The cutting efficiency of the RNP complex was quantified by measuring the frequency of insertions/deletions (INDELs) generated at the on-target site. The median INDEL frequency was 52.6% (range: 27.1-91%) in CB-HSPCs and 68% (range: 31-81%) in PB-HSPCs (FIG. 25C). We confirmed the knock-out efficiency in vivo, following engraftment of mock and RNP treated healthy donor-derived HSPCs. We observed a 4.1-fold and 3.1-fold decrease in the CD3⁺ derived from the RNP-treated condition compared to mock-treated in bone marrow and spleen, respectively (FIGS. 25D and 25E). The predominant INDELs are deletions (2-17 bp) that abrogate RAG2 protein expression (FIG. 25F).

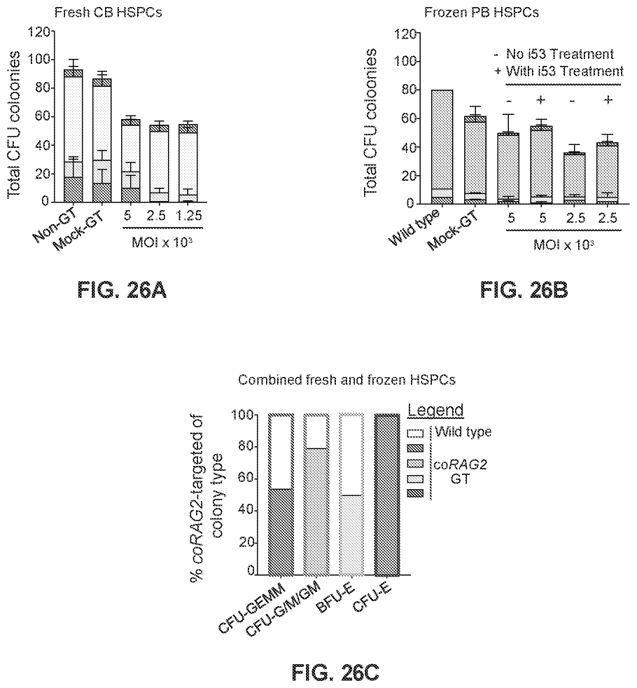

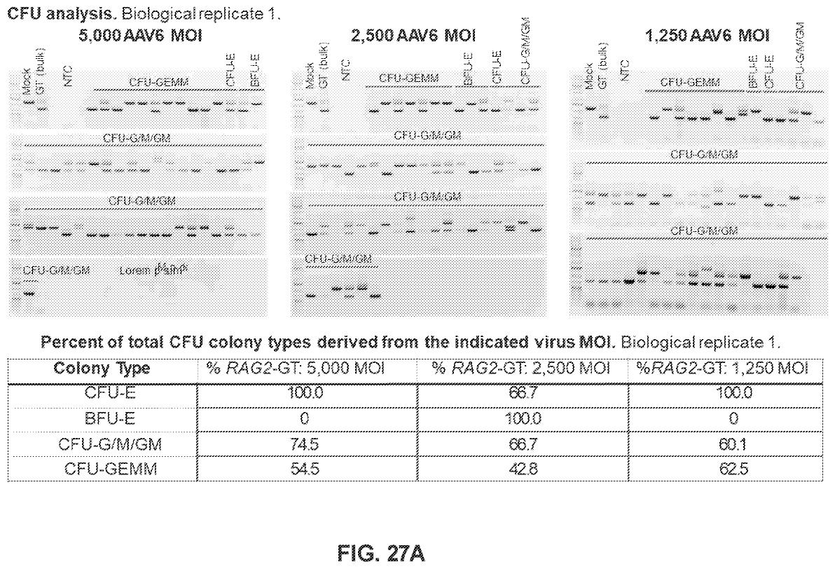

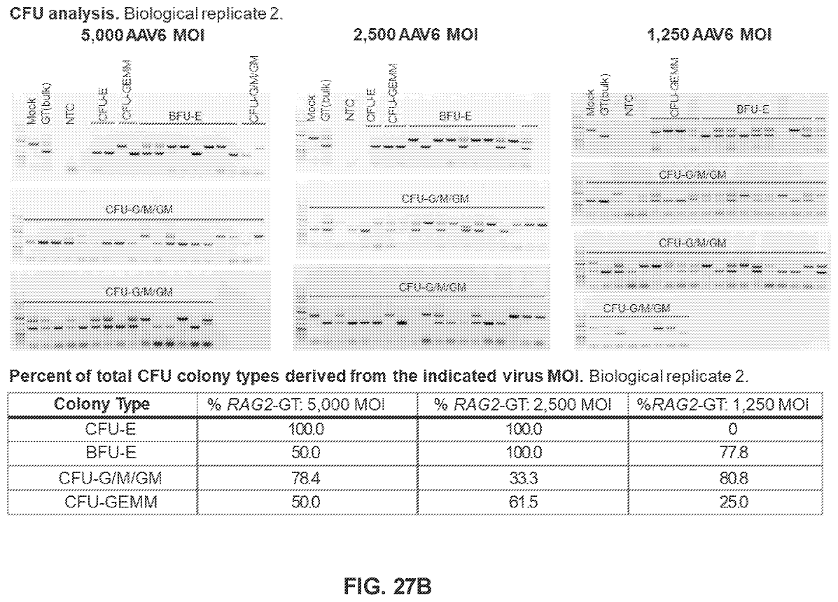

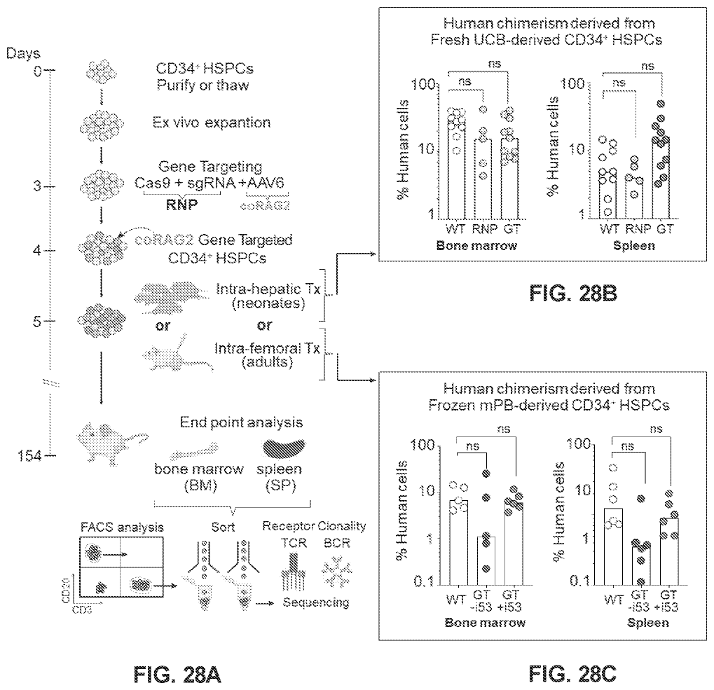

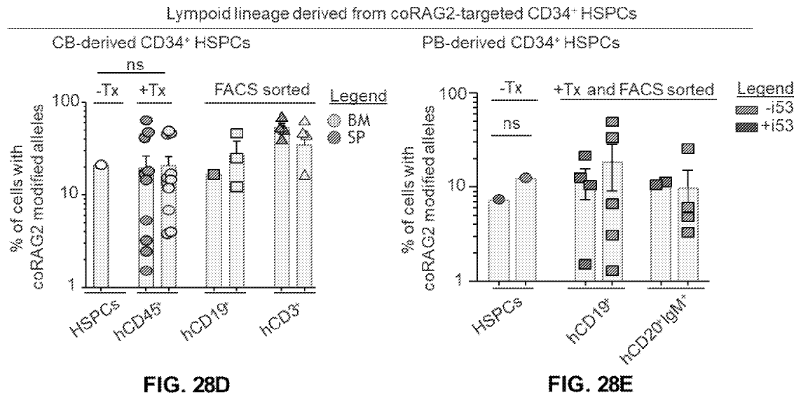

To achieve genomic targeting at the RAG2 locus, we designed a homologous recombination DNA donor corrective template by inserting coRAG2 expression cassette downstream of RAG2 5′ untranslated region (5′ UTR), followed by a woodchuck hepatitis virus post-transcriptional regulatory element (WPRE), and bovine growth hormone polyadenylation (BGH-polyA) signal (FIG. 24A). The corrective RAG2 cDNA donor was codon-optimized to reduce homology to the genomic locus and eliminate unwanted homologous recombination-based integration events while preserving the intact RAG2 amino acid sequences. WPRE was included in the design to increase the transgene expression level if the codon optimization would reduce it. WPRE mechanism of action is not well understood, but it has been proposed to improve transcriptional termination or increase the efficiency of nuclear exports of transcripts. Following RNP electroporation. CB- and PB-HSPCs were transduced with the coRAG2 cassette, and the genomic integration was quantified using digital droplet PCR (ddPCR). As shown in FIG. 24B, the mean percentage of HR-GT cells was 40.6±3 (s.e.m, n=13 unique HSPCs donors). We measured the efficacy of allele modifications in CB-HPSCs by PCR-based genotyping single cell-derived colonies (n=900) from colony formation units (CFU) assay as a function of virus multiplicity of infection (MOI) (FIG. 24C, FIGS. 26A and 26B, FIGS. 27A and 27B). When quantifying the HR-GT in these cells, the frequency of total alleles modified were 58.0% (5.000 MOI, n=5 donors), 56.0% (2,500 MOI, n=4 donors) and 68.7% (1,250 MOI, n=2 donors) (FIG. 28C). Single-cell derived CFU assay also allowed us to estimate the distribution of cells with one (mono-allelic) or two (bi-allelic) alleles targeted in different HSPCs donor samples transduced with different virus MOIs. These analyses indicated that at 5,000 MOI 25.6±2.0 (s.e.m.) and 32.4±8.7 (s.e.m.) had mono-allelic and bi-allelic modifications, respectively, at 2.500 MOI 31.7±4.5 (s.e.m.) and 24.5±7.4 (s.e.m.) had mono-allelic and bi-allelic modifications, respectively and at 1,250 MOI 38.6±14.2 (s.e.m.) and 30.1±4.6 (s.e.m.) had mono-allelic and bi-allelic modifications, respectively (FIG. 28C). The frequency of allele targeting was not significantly different at a lower AAV6 MOI.

RAG2 HR-GT HSPCs gave rise to all four early hematopoietic progenitor cells at comparable frequencies to mock targeted and wild-type HSPCs (FIGS. 26A and 26B). Notably, the HSPCs targeted with the coRAG2 cDNA differentiate into myeloid and erythroid progenitors, demonstrating that unintended perturbations were not introduced in the non-lymphoid lineages (FIG. 26C). To determine if you increase the frequency for HR-GT at lower AAV6 MOI, we tested p53 binding protein 1 (53BP1) inhibitor (153) previously shown to regulate non-homologous end joining (NHEJ) DNA repair pathway. We found that 53BP1 inhibition resulted in a 1.5-fold increase in homology direct repair (HDR) without diminishing the modified cells' differentiation potential, in line with our recent publication (FIG. 26B).

Overall, these data show that the coRAG2 genome targeting protocol is reproducible, highly efficient in human CB and PB-HSPCs, and preserves the cells' ability to generate early hematopoietic progenitors in vitro.

### Specificity and Safety of the RAG2 Genome Correction Approach

To run a stringent and comprehensive off-target profile of our RAG2 guide (sgRNA-3), we combined in silico prediction of closely matched sites using COSMID (CRISPR Off-target Sites with Mismatches, Insertions, and Deletions), a bioinformatics-based tool with unbiased genome-wide analysis, GUIDE-seq. Off-target activity at a total of 48 predicted loci was quantified by deep sequencing in the genome of PB-derived HSPCs from a RAG2-SCID patient carrying compound heterozygous missense mutations (c.[296C>A: 1342C>A]) (FIG. 24D). We compared the percent INDELs detected in mock and cells electroporated with the HiFi S.p. Cas9 variant. Sites that were considered true off-target (OT) were those where the percent INDELs was >0.1% (limit of detection) and were higher in the RNP-treated condition compared with the mock sample. Based on these criteria, none of the sites were valid off-targets (FIG. 24D, Table 1). Table 1 shows a description of off-target (OT) sites above the limit of detection (>0.1). In Table 1, mismatched bases from RAG2 guide sequence are bolded and underlined, and the PAM sequences are italicized. When normalized to mock, all % INDELs are <0.1 using high fidelity (HiFi) Cas9 protein. Distance from transcription start site is shown in base pairs. For the OT-11 and OT-14 sites, the frequencies of INDELs were 0.15 and 0.16, respectively. When normalized to the percent INDELs detected in the mock sample at these two sites, the frequency of INDELs was abolished entirely. The on-target efficacy was quantified at >70%. These results suggest that our RAG2 guide (sgRNA-3) combined with HiFi S.p. Cas9 generates no detectable off-target activity during the five days of ex vivo culture and editing in RAG2^{-/-} patient-derived HSPCs.

TABLE 1
Description of off-target (OT) sites above limit of detection (>0.1).

| | | % INDELS | | | | |
|---|---|---|---|---|---|---|
| Target | Sequence | Mock | RNP | Feature | Distance | Gene type |
| RAG2 | T - G - C - A - G - | 0.05 | 71.9 | Exon | −6 | protein- |
| | A - G - A - C - A - | | | | | coding |
| | T - A - G - T - T - | | | | | |
| | T - C - T - G - A | | | | | |
| | TGG | | | | | |
| OT-11 | T - G - C - A - T - | 0.1 | 0.15 | Intergenic | −382,355 | non-coding |
| | A - G - T - C - A - | | | | | |
| | T - T - G - T - T - | | | | | RNA |
| | T - C - T - G | | | | | |
| | - A TGG | | | | | |
| OT-14 | T - A - A - A - G - | 0.2 | 0.16 | Intronic | −120,475 | non-coding |
| | A - G - A - C - A | | | | | |
| | - T - A - T - T - | | | | | RNA |
| | T - T - C - T - G | | | | | |
| | - A GGG | | | | | |

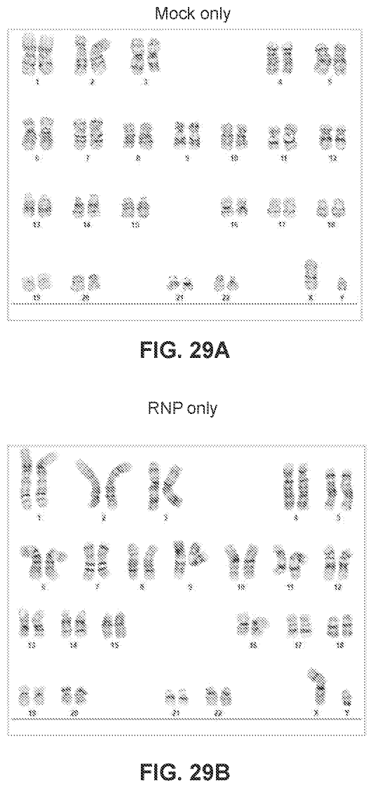

We performed karyotype analysis on the same RAG2/patient-derived HSPCs to assess whether genomic instabilities were generated by ex vivo culture, nucleofection, and RNP-treatment. Whole chromosomal analysis was carried out on 20 cells from mock and RNP-only conditions and detected no chromosomal abnormalities in either of the condition (FIGS. 29A and 29B).

Cumulatively, we performed 63 dissections on mice engrafted for 20-weeks with a total of 28 million genome-modified HSPCs [NHEJ genome-edited and HR genome-targeted HSPCs (10 and 4 mice with RNP from healthy donors and RAG2″ patient, respectively and 17 and 32 with HR-GT from healthy donors and RAG2′ patient, respectively)] and no gross tumors were present.

The absence of off-target activity of the RAG2 guide (sgRNA-3) and the lack of tumorigenicity offers the first line of evidence, at the pre-clinical level, for the safety of our genome correction strategy for RAG2 deficiency.

Human Hematopoietic System is Derived from coRAG2-HSPCs

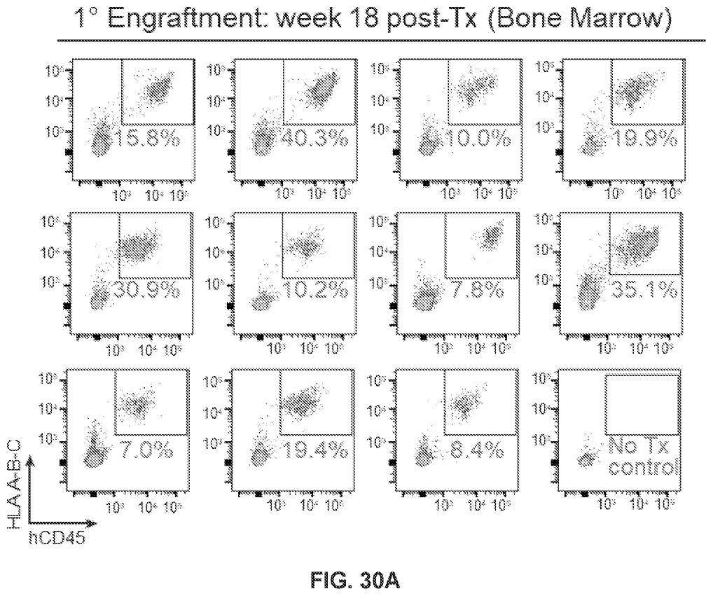

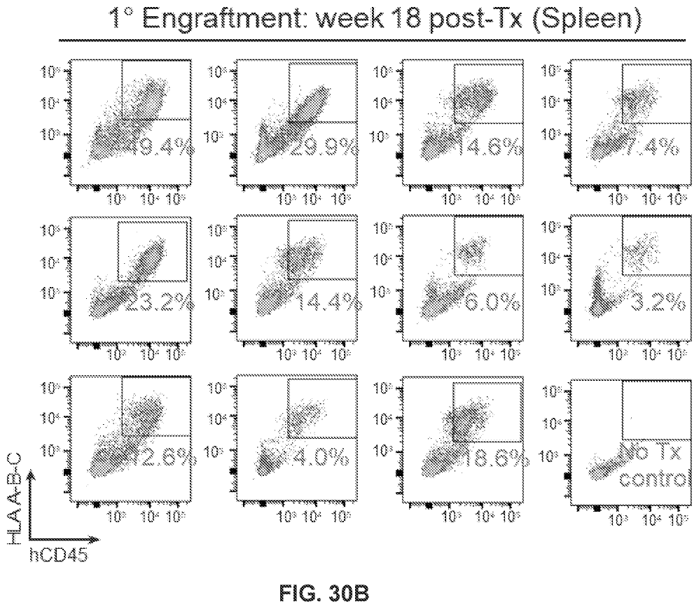

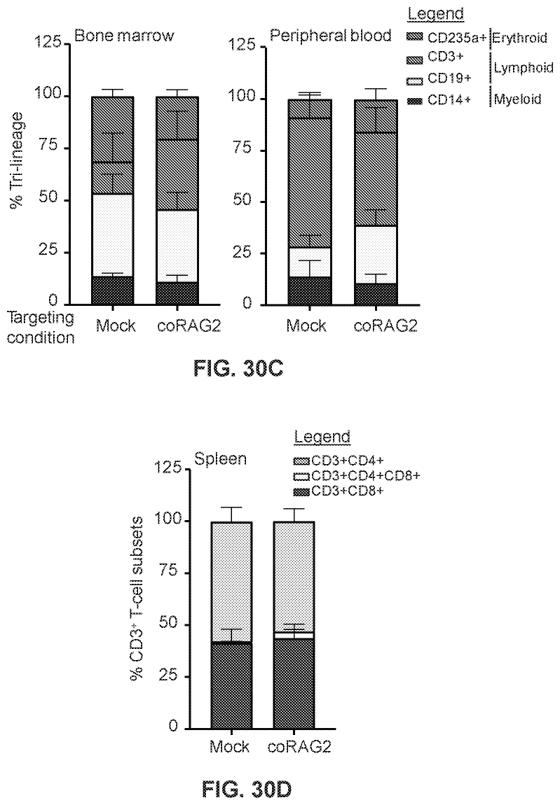

To determine if HSPCs targeted with a coRAG2 can support long-term engraftment and tri-lineage differentiation, we performed engraftment studies (FIG. 28A) into NOD-SCID-gamma (NSG) and NSG expressing stem cell factor (SCF), granulocyte-macrophage colony-stimulating factor (GM-CSF), and interleukin-3 (IL3)-NSG-SGM3 mice. We first tested the hematopoietic reconstitution of coRAG2-GT in fresh CB-HSPCs using intra-hepatic injections into 2 days old NSG pups because the IH system has been shown to best support lymphopoiesis. An equal number of unmodified (wild type and mock), RNP-treated, and HR-GT CB-HSPCs (2.5×10⁵) were transplanted intra-hepatic into sub-lethally irradiated 2 days old pups. Primary human engraftment was measured 18 weeks post-transplant by FACS quantifying the percent of bone marrow (FIG. 28B, left panel. FIG. 30A) or spleen (FIG. 28B, right panel. FIG. 30B) cells expressing both human CD45 and human leukocyte antigens (HLA-ABC) out of total mouse cells. The percent frequency of bulk allele targeted before engraftment was 21.0%. Post-engraftment, the median frequencies of hCD45*/HLA-cells in the bone marrow were as follows: unmodified 28.6% (range: 10.3-39.4%), RNP 15.4% (range: 4.4-41.8%), 15.8% (range: 7.0-40.3%). These results showed no statistically significant difference in repopulation capacity in cells that underwent GT-HR or GE-NHEJ compared to cells that did not (mock or wild type). The mean frequency of human cells expressing coRAG2 purified from bone marrow (BM) was 19.5±6.5 (s.e.m.; n=11) and from spleen (SP) was 20.7±5.3 (s.e.m, n=11). Human cells carrying the coRAG2 cDNA were also found in sorted lymphocytes (CD19⁺ B cells and CD3⁺ T cells) from bone marrow (FIG. 28D, yellow squares, and circles) and spleen (FIG. 28D, red squares, and circles). Our quantification only shows a proliferative advantage in T cells derived from bone marrow 53.5±6.1 (s.e.m, n=4) with the remaining sorted cells expressing coRAG2 at no statistical difference from the bulk (not sorted) human hCD45⁺/HLA⁺ cells: 16.6±5.3 (s.e.m, n=1; sorted CD19⁺ from BM), 34.4±11.5 (s.e.m., n=5; sorted CD3⁺ from SP), 27.9±9.9 (s.e.m., n=3; sorted CD19″ from SP).

We repeated the xenotransplantation assays described above, using healthy donor-derived frozen mobilized peripheral blood (PB) CD34⁺ HSPCs (FIG. 28C), to assess the efficacy of our gene correction strategy using a cell source of similar derivation as of RAG2 patients. An equal number of unmodified (wild type and mock) and HR-GT CB-HSPCs (500,000) treated (+i53) or not (−i53) were transplanted intra-femoral into sub-lethally irradiated 8 weeks-old NSG-SGM3 mice (FIG. 28C). Primary human engraftment was measured, as described above, 22 weeks post-transplantation. The median frequencies of hCD45⁺/HLA⁺ cells in BM were as follows: unmodified 6.8% (range: 4.0-14.2), GT-HR (−i53) 1.12% (range:0.2-25.0). GT-HR (+i53) 5.9% (range: 3.6-11.4). We observed no statistically significant difference in repopulation capacity in cells that underwent GT-HR compared to cells that did not, nor among GT-HR cells treated or not with p53 inhibitor (153), in bone marrow (FIG. 28C, left panel, data not shown) or spleen (FIG. 28C, right panel). Human cells carrying the coRAG2 cDNA were found in sorted mature B cells: CD19⁺ [−i53: 11.5±4.1 (s.e.m., n=4), +i53: 18.8±9.6 (s.e.m., n=5)] and CD20⁺ IgM⁺[−i53: 10.9±0.4 (s.e.m., n=2), +i53: 9.9±5.2 (s.e.m., n=4) showing that coRAG2 cDNA drives productive V(D)J recombination reactions (FIG. 28E).

To assess the GT-HR cells' ability to differentiate into multiple hematopoietic lineages, we used human CD3, CD19, CD14, and CD235a markers to identify the lymphoid (B, T), myeloid (monocytes), and erythroid, respectively. Compared to unmodified (mock) cells, coRAG2 modified cells showed no statistical difference skewing towards a particular lineage in bone marrow (FIG. 30C left panel), in circulating cells in the peripheral blood (FIG. 30C right panel) or spleen (FIG. 30D left panel, FIG. 31).

Together, these data demonstrate that genome targeting process and coRAG2 expression cassette support efficient and durable hematopoietic engraftment, regardless of the source of healthy donor HSPCs used, and can drive lymphoid development to mature cells stage, in vivo, without disrupting other hematopoietic lineages.

In Vivo B-Cell Lineage Reconstitution from Gene-Corrected RAG2^{null} HSPCs

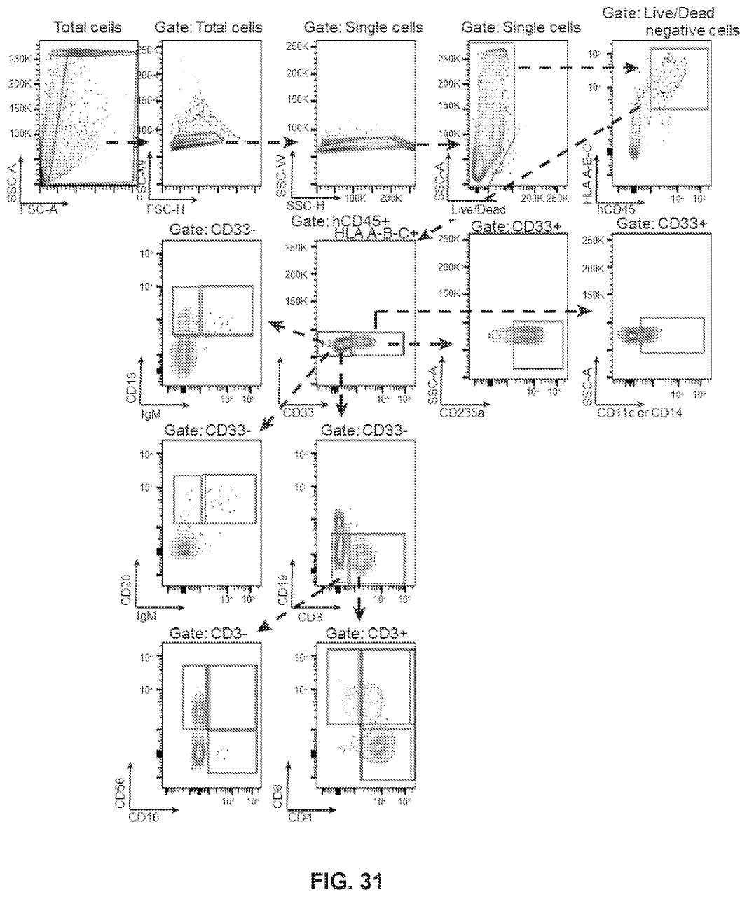

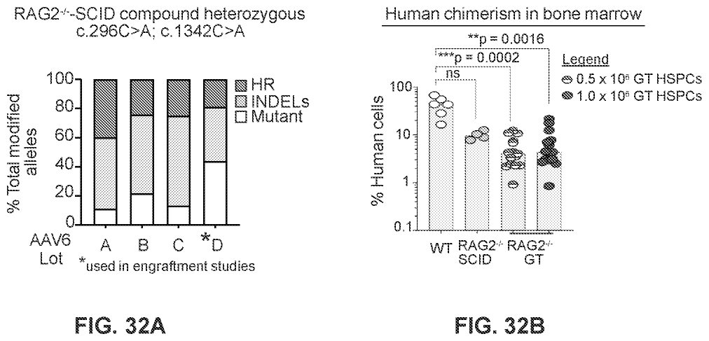

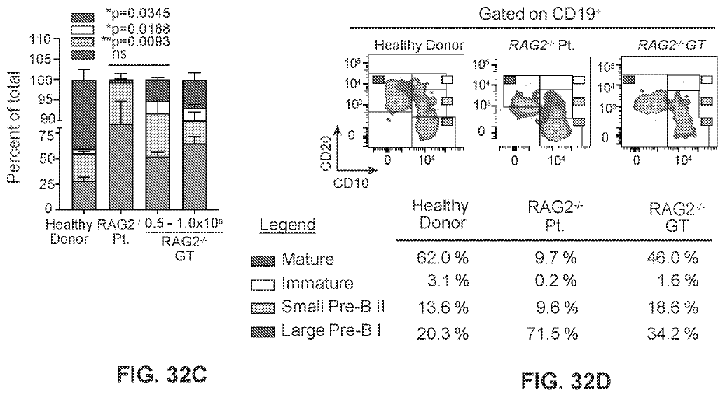

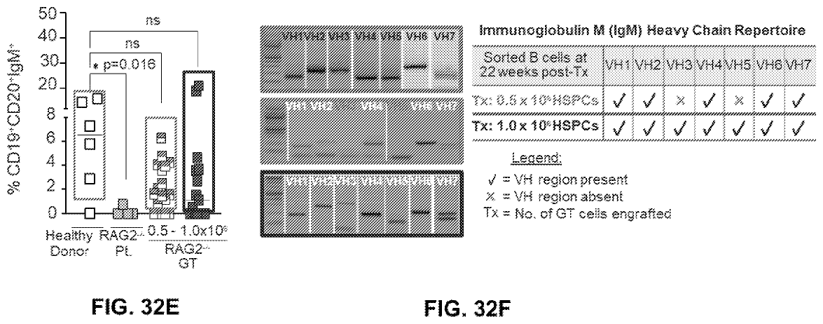

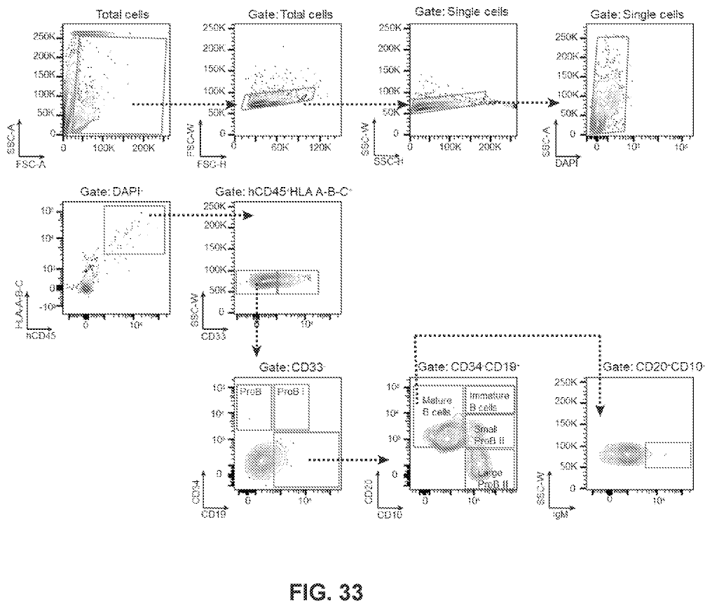

To assess the efficacy and potency of our therapeutic approach to phenotypically correct the B-cells developmental block present in a typical RAG2-SCID patient, we used our CRISPR/HiFiCas9-AAV6 to insert coRAG2 transgene at the RAG2 locus in RAG2 patient-derived HSPCs, carrying a heterozygous compound mutations c.[296C>A: 1342C>A]. Clinical data for this RAG2-SCID patient was confirmed. We tested four different non-GMP (good manufacturing product) grade AAV6 virus batches, using 5,000 MOI, on the same frozen PB-HSPCs derived from the same RAG2-SCID patient (FIG. 32A). Total genomic modification—HR (green bars) and INDELs (grey bars)—generated by three different virus batches in the absence of i53 treatment showed a mean % INDEL-GE of 54.9±3.7 (s.e.m.), mean % HR-GT of 29.9±5.1 (s.e.m.), respectively, with 15.2% alleles (+5.6 s.e.m.), remained unmodified. These viruses, however, decreased cellular viability, diminished CFU, and long-term engraftment potential of genome modified RAG2^{-/-} HSPCs (data not shown). We have, therefore, purified a fourth AAV6 batch and used it to transduce the patient cells at lower MOI (2,500) and in the presence of p53 inhibitor (+i53). Under these optimized conditions, we achieved a % HR-GT of 19.2 and % INDEL-GE of 37.0. The level of HR-GT we obtained in RAG2^{-/-} patient HSPCs was comparable to that obtained in healthy donors HSPCs that were engrafted. One million or 500.000 HR-GT HSPCs were transplanted intra-femoral into 6-8 weeks old NSG-SGM3 mice, along with unmodified controls (mutant and healthy donor) HSPCs. Human chimerism was quantified at 22-weeks post-transplant. In bulk transplants, the median human chimerism in the bone marrow was as follows: 4.4% (range: 0.9-21.7) in mice transplanted (Tx) with 1 million HR-GT, 4.1% (range: 0.9-12.3) in mice transplanted with 500.000 HR-GT, 9.6% (range: 7.78-12.5) in mice transplanted with 1 million unmodified mutant HSPCs and 43.5% (range: 16.4-67.3) in mice transplanted with 1 million unmodified healthy donor HSPCs (FIG. 32B). A two-fold decrease in human engraftment was observed between the healthy donor and HR-GT HSPCs, regardless of the number of cells transplanted. Still, no statistically significant difference was noted between the engrafted healthy donor and unmodified patient-derived HSPCs, suggesting an increased sensitivity of RAG2^{-/-} patient-derived HSPCs to the genome correction reagents. Transplanting NSG-SGM3 mice with 500,000 HR-GT HSPCs generated a statistically increase fraction of small pre-B II (light blue bar) cells, immature (white bar), and mature (burgundy bar) cells when compared to uncorrected mutant cells (FIGS. 32C and 32D). The triple-positive CD19 CD20⁺ IgM⁺ mature B cells derived from HR-GT HSPCs (FIG. 32E, red/white and black squares) were produced at no statistical difference from healthy donor engrafted cells (FIG. 32E, white squares). When sorted (sorting scheme described in FIG. 33) and analyzed for IgM immunoglobulin heavy chain repertoire, the HR-GT engrafted at 1 million cells per mouse showed expression of all 7 heavy chain variable regions in (FIG. 32F, black bottom panel). In comparison, the HR-GT engrafted at 500,000 cells per mouse expressed 5 out of 7 heavy chain variable regions in the (FIG. 32F, middle red panel).

Together, this data indicates that our RAG2-specific gene correction platform has the potential to modify up to 84.8% of mutant alleles, of which it corrects up to 29.9% and disrupts up to 54.9%. Furthermore, genome-modified RAG2-SCID HPSCs can sustain long-term engraftment, and a genomic correction of 19.2% was sufficient in restoring the B-cell development block in a typical RAG2-SCID HSPCs.

RAG2^{null} gene-corrected HSPCs rescues T-cell development in NSG-SGM3 mice

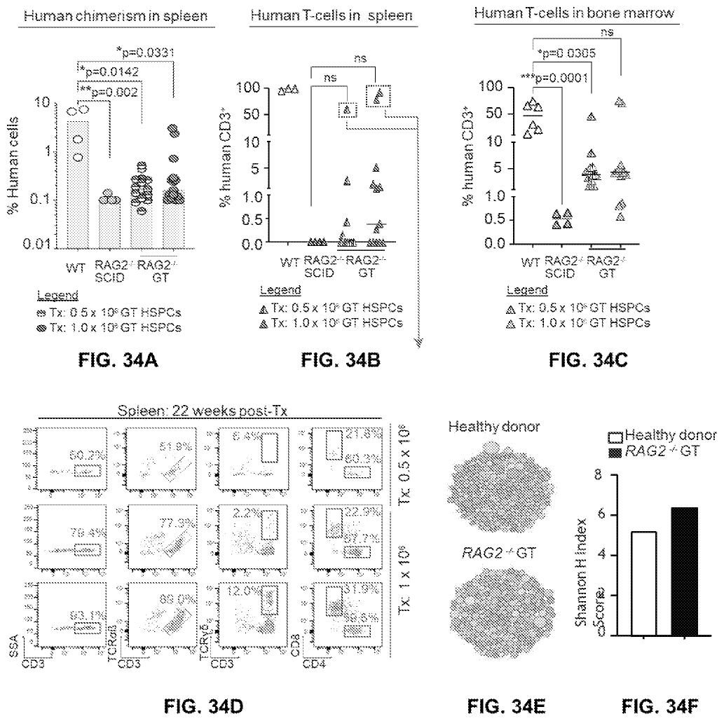

Since we observed correction in the B-cell compartment, we also assessed the potential of our gene-corrected RAG2-SCID HSPCs in rescuing the T-cell developmental defect. Human chimerism in the spleen was lower than in the bone marrow: 0.2% (range: 0.1-3.0) in mice transplanted (Tx) with 1 million HR-GT, 0.2% (range: 0.06-0.5) in mice transplanted with 500,000 HR-GT, 0.1% (range: 0.1-0.14) in mice transplanted with 1 million unmodified mutant HSPCs and 4.3% (range: 0.8-7.4) in mice transplanted with 1 million unmodified healthy donor HSPCs (FIG. 34A). HR-GT HSPCs engrafted in bone marrow, and spleen gave rise to CD3⁺ T-cells at levels comparable to healthy donor-derived T-cells (in 3 mice) (FIGS. 34B and 34C). Using FACS-based analysis of the spleen-derived T-cells (FIG. 34B, dotted boxes and FIG. 34D), we showed that the CD3⁺ T-cells express αβ and γδ T-cell receptors (TCR) and gave rise to single-positive CD4⁺ and CD8⁺ T-cells, demonstrating that RAG2^{-/-} corrected HSPCs can progress through the double-positive (DP) developmental stage and achieve maturation.

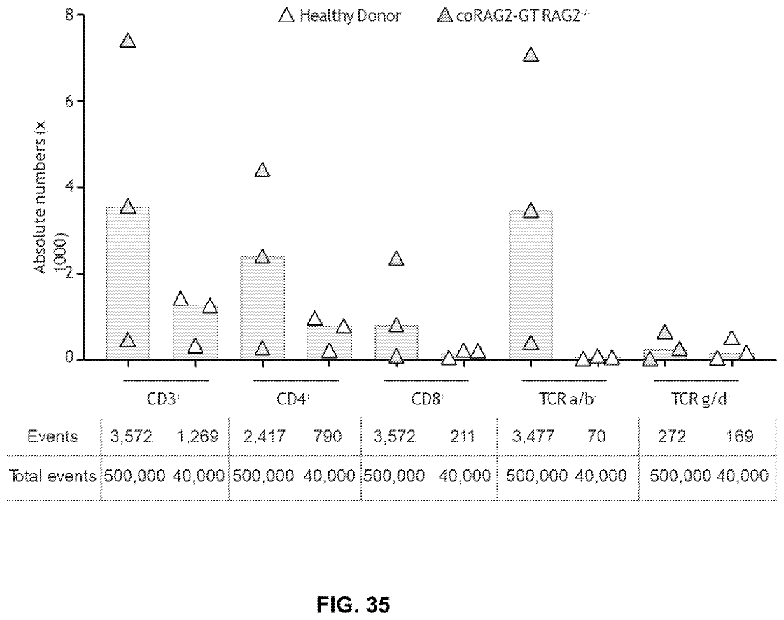

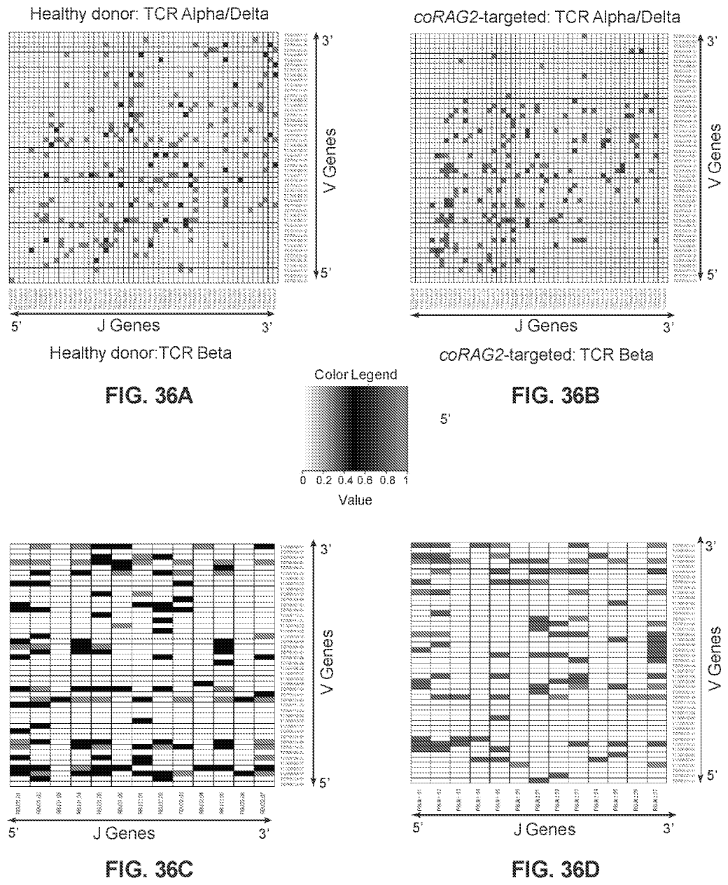

To further assess the degree of T-cell development correction, we used high-throughput immunoSEQ to quantify the TCR repertoire to determine the clonality at the complementary determining region 3 (CDR3). CDR3 is the region that promotes TCR-peptide MHC complex formation. Using a treemap profile analysis of the CDR3 of sorted HR-GT derived CD3⁺ T cells and healthy donor control, we determined an oligoclonal pattern of TCR CDR3 specificity, with Shannon's H entropy index of 6.4 and 5.2, respectively (FIGS. 34E and 34F). In addition, virtual spectratyping analysis revealed CDR3 lengths of productive rearrangements in bulk T-cells from GT-HR and healthy donor control (FIG. 35).

It has been reported that mature T cells derived from RAG^{-/-} patients with hypomorphic mutations, that have residual RAG protein expression and therefore limited V(D)J activity, have an abnormal TRA repertoire composition that is defined by a pronounced usage of the distal-most TRAV and TRAJ genes [63]. To determine if our level of gene correction achieved sufficient level RAG2 expression supportive of normal levels of V(D)J recombination activity, we used immunoSEQ to assess the quality of TCR rearrangements at the TCRB (TRB) and TCRα/TCRγ (TRA/TRD) loci. The HR-GT RAG2-SCID derived T cells showed a comparable rearrangement pattern of the TRAV and TRAJ in bulk CD3⁺ T-cells compared to CD3⁺ T-cells derived from healthy donor control.

Combined, our data show that coRAG2 cDNA can restore functional RAG complex following genomic integration, promoting T-cell development with a normal VJ pairing in genome corrected cells, supporting our previously published in vitro results.

### Correction of the Immature NK CD56^{bright} Phenotype in RAG2-SCID Patient

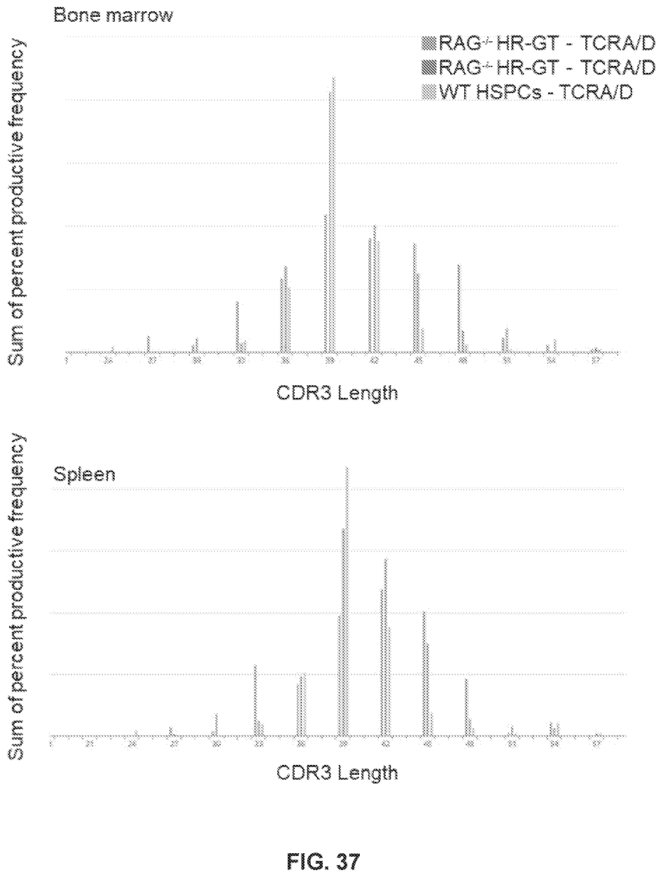

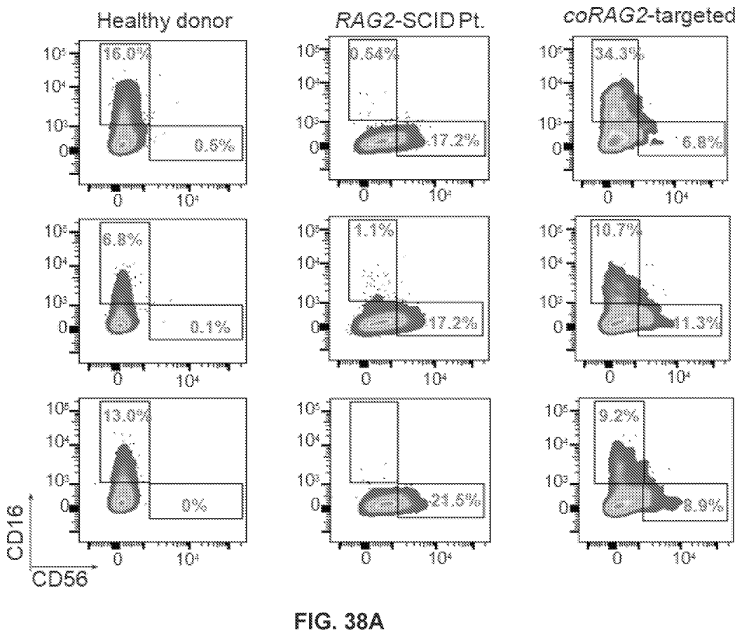

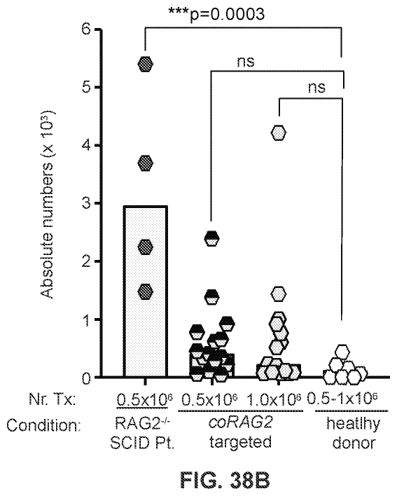

It was reported that RAG deficient mice display an increased proportion of NK cells with heightened cytotoxic activity and limited survival potential [64]. These observations were also reported in humans, in SCID patients [65], caused by defects in RAG and NHEJ genes. We characterized the NK population derived for genome corrected RAG2-SCID-derived HSPCs transplanted into NSG-SGM3 mice. Our FACS analysis of the human cells purified from mouse bone marrow confirmed that NK cells derived from uncorrected RAG2-SCID HSPCs expressed significantly higher levels of CD56^{bright}CD16⁻ cell surface markers compared to healthy donors (FIG. 37, top panel). Following genome correction, the CD56^{bright}CD16⁻ population was reduced to levels comparable to healthy donors (FIG. 37, bottom panel).

Together, these results show that our levels of genome correction extend beyond the lymphoid lineage defect of the RAG2-SCID deficiency to that of natural killer cells, which display an immature phenotype.

### DISCUSSION

We describe the development of a novel therapy for correcting a form of SCID caused by loss of function mutations in the RAG2 gene. Through a comprehensive set of preclinical studies performed in vitro, in vivo, in immunodeficient mice, using healthy donor and RAG2-SCID patient-derived CD34⁺ HSPCs, we demonstrated the efficient application of CRISPR/Cas9-AAV6 technology to express RAG2 from the endogenous locus, to promote V(D)J recombination and ultimately lymphoid lineage development. For primary immunodeficiencies (PIDs) like RAG2-SCID, where a strict spatiotemporal level of gene expression and regulation is necessary to achieve normal immune development and function [38, 66], a gene correction approach has several therapeutic and safety benefits when compared to semi-random gene addition strategy: (1) it restricts the gene activity to the lymphoid lineage and to the G₀/G₁ cell cycle phase [safety], which in turn (2) it circumvents the risk of genotoxicity from ubiquitous RAG activity [safety], (3) it allows for physiological expression [therapy], (4) it is easily adaptable to correct other forms of RAG2^{-/-} diseases, such as OS, AS and CID-G/AI for which disrupting and correcting the mutant alleles both alleviate disease burden and achieve clinical levels of correction [therapy].

To date, no published data demonstrated the feasibility of restoring expression, regulation, and function of RAG2 in vivo, using patient-derived HSPCs, to reinstate V(D)J activity and normal lymphoid lineage development. Our design is based on inserting the RAG2 full open reading frame and an exogenous polyadenylation signal (polyA) downstream of the transcription start site. This approach achieved genome modification (INDELs and HR) in up to 85.0% of alleles, of which over 50% of mutant alleles are disrupted, and 30% are corrected. Follow-up allo-HSCTs studies show that a minimum of 20% donor chimerism is necessary to achieve clinical correction in RAG—SCID patients, suggesting that our levels of genomic modification achieve and surpass the requirement for attaining clinically relevant levels of correction. Moreover, RAG^{-/-} patients with OS, AS, and CID-G/AI indications would also benefit from the high frequency of alleles disruptions since for these patients' mutant alleles retain residual protein activity, promoting a dysregulated immune development. Thus, both the HR- and NHEJ-mediated genome modification that we report for the RAG2 locus can contribute significantly towards reconstituting a normal adaptive immune system, which would extend beyond correcting the loss of function mutations.

Using in silico prediction algorithm combined with genome-wide in vivo analysis, we confirmed the absence of detectable off-targeted activity when using our lead RAG2 guide complexed with high fidelity s.p. Cas9 nuclease. The conclusion that our novel genome correction therapy is safe is further supported by the lack of tumorigenicity in 63 mice transplanted with 28 million genome-modified HPSCs (3.5 million modified cells derived from healthy donors and 24.5 million modified cells derived from RAG2-SCID patients) analyzed after 18-22 weeks, with no observed hematopoietic skewing. Furthermore, we showed normal karyotyping in cells modified with the guide.

Analysis of the self-renewal and multi-lineage differentiation capacity of the RAG2 modified cells demonstrates that our genome correction approach can modify hematopoietic stem cells with long-term repopulating potential. We observed no statistical difference in the long-term engraftment capacity in the initial experiments when using healthy donor-derived HSPCs, and when comparing unmodified with HR-GT modified bulk engrafted or sorted human cells. Furthermore, there was no decrease in the frequency of HR-GT alleles pre- and post-transplantation. This is an important observation for future therapeutic applications. Not only do we attain clinically relevant levels of allele correction, but the modified cells preserve their long-term self-renewal and repopulating capacity, as measured by the current gold-standard xenotransplantation studies.

In transplantation studies using RAG2-SCID patient HSPCs, cells that underwent HR-GT had a 2-fold lower long-term engraftment potential compared to unmodified, mutant cells. The observed lower engraftment could represent an adverse effect of the non-GMP grade AAV6 virus used. We have tested four different virus batches and observed a significant decrease in the CFU potential, in addition to a reduction in cellular viability, 48 hours following virus transduction. Since this decrease in long-term hematopoietic potential was not observed when using healthy-donor HSPCs, we cannot exclude the possibility that the RAG2′ patients' hematopoietic progenitors (HPCs) and/or stem cells (HSCs) have reduced regenerative potential. In support of this, unmodified RAG2-SCID HSPCs show a 4.5-fold lower engraftment capacity when compared to healthy-donor-derived HSPCs transplanted in the same cohort of mice. Another possible explanation is that in the absence of RAG expression in patient cells, known to first occur in the early lymphoid progenitors, cells with lower DNA repair capacity are not selected against, resulting in an overall decrease in the fitness of the HPCs, necessary for supporting HSCs engraftment after conditioning [65].

We examined the potential of genome-corrected patient cells to overcome the lymphoid development block that defines a typical RAG2-SCID disease. Engraftment of the coRAG2 HR-GT HSPCs led to B-cell development rescue when 500,000 corrected HSPCs were transplanted into immunodeficient mice. Our analysis showed a decrease in the large pre-BI cells with a subsequent increase in small pre-B II, immature and mature B cells. Patient-derived corrected and engrafted into mice generated immunoglobulin M (IgM) with a repertoire composed of all seven heavy chains, at no statistical difference from healthy donor-derived B-cells. We did not detect immunoglobulin G (IgG) in the corrected and sorted mature B cells, possibly due to the absence of a foreign antigen challenge to stimulate class switch recombination (CSR) in immunodeficient mice housed under pathogen-free conditions. When translating results from experimental humanized mouse models to clinical settings, we must acknowledge that the microbiome of mice and humans is different, and it plays a crucial role in shaping the immune system development and function [67]. Humanized mice models have an underdeveloped immune system, which could underestimate the potency of the therapeutic product we are testing.

T-cells were also generated from genome corrected-patient derived HSPCs. They were detected in the spleen and bone marrow. In the latter, T-cells were generated in mice transplanted with either 500,000 or 1 million corrected HSPCs. However, a more robust T-cell development was observed when a higher cell dose was delivered to mice. One explanation for this observation is that when a lower threshold of genome correction (19.0%) is attained, fewer corrected early thymic progenitors (ETP) survive the mouse thymic selection. Delivering a higher dose of corrected cells could compensate for this limitation. However, we showed that the ETP that survived the thymic selection developed into mature lymphocytes, expressing αβ and γδ T-cell receptors that can propagate developmental signals to drive differentiation into single-positive CD4⁺ and CD8⁺ T-cells. The T-cells' receptors developing from patient-derived RAG2 genome-corrected HSPCs and healthy donors both showed an oligoclonal rearrangement pattern at the TRA TRD locus with a similar pattern in TRAV usage and TRAJ genes. Our previous publication demonstrated a polyclonal TCR repertoire in CD3⁺ T-cells derived from patient-derived RAG2-SCID genome corrected iPSC differentiated in the artificial thymic organoid (ATO) system. One possible explanation for this observed discrepancy is the consideration that of the genome corrected and transplanted HSPCs clones, only a limited number repopulated the mouse thymus, which restricts the number of stem cells that can contribute to the T-cell lineage development. This is a viable hypothesis confirmed by a recent study, whereby using lentivirus cellular barcoding and purified human BM- or CB-derived HSPCs transplanted into NSG mice, it was shown that less than 10 HSC clones contributed to the repopulation of the mouse thymus microenvironment [68]. Although the study demonstrated that even with a limited clonal contribution to the T-cell lineage, a diverse and polyclonal TCR repertoire can be generated [68], we reason that the fresh CB- or BM-derived HSPCs used in their study holds greater lymphoid potential than frozen PB healthy or patient-derived HPSCs, used in our study.

In the absence of myeloablative conditioning, RAG-deficient patients show a high graft failure rate, and those that engraft have poor lymphoid reconstitution [24]. It is known that genetically defective early common lymphoid progenitors (CLPs) occupy the bone marrow and thymic niches [50]. In the absence of niche cleaning, the donor functional CLPs must compete with the endogenous genetically defective progenitors for space to engraft and develop. In addition, natural killer (NK) cells, which are also derived from CLPs [69, 70], occupy the bone marrow niche. It has been shown that the RAG-patient derived NK cells have higher perforin expression and increased degranulation potential [64, 71]. These cytotoxic NK cells can attack the graft in the absence of conditioning. We show that our genome correction strategy shifts the predominant NK population from a CD56^{bright} CD16⁻ to CD56^{dim} CD16⁺, at frequencies no different from healthy donors. These results show that an autologous-HSCT using genome corrected HPSCs, combined with reduced intensity myelosuppressive conditioning, would conceivably provide therapeutic benefits by eliminating the competitive environment in the hematopoietic niches and assuring graft survival through correction of the cytotoxic function of the derived NK cells.

In our pre-clinical studies, we used p53 inhibitor (153) as part of our genome editing protocol to reduce the concentration of the non-GMP virus used during transduction without decreasing the HR frequency. Treatment with i53 resulted in ˜ 1.5-fold higher genome targeting (HR-GT) without increasing engraftment potential. We anticipate that a highly purified GMP-grade virus would eliminate the toxicity observed from a non-GMP-grade virus, thus eliminating the need for i53 treatment and simplifying the path to clinical translation. Our data establish the first in vivo study to show phenotypic correction of the lymphoid lineage defect in a typical RAG2-SCID patient-derived HSPCs, using CRISPR/Cas9-AAV6 platform, and provides the rationale for further development and testing of this strategy in the treatment and characterization of the autoimmune manifestations' mechanisms in RAG2^{-/-} diseases.

### Experimental Procedures

SgRNA Guide and HiFi s.p. Cas9

CRISPOR software (crispor.tefor.net) was used to select candidate RAG2 sgRNAs. Guides were synthesized by Synthego Corp (Redwood City, CA. USA) as a chimeric 100 nucleotide sgRNA, and chemically modified using a proprietary formulation. Lead RAG2 sgRNA guide (RAG2-sg3: 5′-TGCAGAGACATAGTTTCTGA-3′) was purchased from TriLink BioTechnologies (San Diego, CA, USA), as 2′ O-methyl 3 phosphorothioate and HLPC-purified. High fidelity (HiFi) s.p.Cas9 nuclease protein was purchased from Integrated DNA Technologies (Coralville, IA, USA).

### Electroporation and Transduction of Human Stem Cells

RNP complex was generated by mixing HiFi Cas9 with sgRNA at a molar ratio of 1:5 (450 ug/ml HiFi Cas9 protein with 960 ug/ml of sgRNA from Trilink), at 37ºC for 30 minutes prior to nucleofection. Nucleofection was carried out in a 16-well nucleofection strip, using Lonza Nucleofector 4D, program DZ-100, with 20 ul of P3 solution. 1×10⁶ to 3×10⁶ HSPCs were nucleofected in one well of a 16-well strip. Following nucleofection, cells were transduced, as we previously described [16]. AAV6 was added at 5.000 MOI unless otherwise stated. Mock control did not receive RNP complex prior to nucleofection.

rAAV6 Donor Design and AAV6 Virus Purification

The RAG2 donor vector was constructed by PCR amplifying 400 bp left and right homology arms, flanking the RNP cut site, for the RAG2 locus from human CD34⁺ genomic DNA. BGH polyA and WPRE sequences were amplified from plasmids. The donor plasmid was constructed using Gibson cloning New England Biolabs (Cat #E5510S) into a pAAV-MCS plasmid containing AAV2-specific inverted repeats (ITR) Stratagene (Santa Clara, CA, USA). Corrective, codon diverged RAG2 cDNA was designed by GeneScript (Piscataway, NJ, USA) with silent mutations generating 75% homology to the endogenous gene. AAV6 virus was purified, as we previously described [16].

### Human CD34⁺ HSPCs

Frozen mobilized peripheral blood (PB) CD34⁺ HSPCs were purchased from AllCells (Alameda, CA. USA) and thawed as previously described [72]. Fresh CB-derived CD34⁺ HSPCs were obtained under informed consent from Binns Program for Cord Blood Research at Stanford University, as purified and cultured as previously described [16].

### Genome Editing (INDELs) and Genome Targeting (HR) Quantification

Insertions and deletions (INDELs) frequencies were quantified using TIDE or ICE (Synthego) online software on genomic DNA extracted using Quick Extract. Epicentre (Madison. WI, USA), and amplified using F (5′ ATGTGGTTCTTTCAGCTG 3″) and R (5′ CGAAAAGTAACCTTTTTGTTGT 3′) primers. Genomic integration was quantified by droplet digital PCR (ddPCR), as we previously described [16]. To detect insertions at the RAG2 genomic locus we used F-5′ TCT CAC CTC CCA TTC CCT AG 3′. R-5′ TCA GGG CGA TAT TGT TGG AC, and labeled probe F-5′ FAM/CCC GTC TAG/ZEN/TCA CTT CGC ACC TTC GGC/3IABKFQ 3′. The reference assay designed to detect the RAG1 reference genome sequence is: F-5′ GCACAGGAAGTTTAGCAGTG 3′. R-5″ GGGAATTCAAGACGCTCAGA 3′, and probe 5′ HEX/CCC GAG GAA/ZEN/CGT GAC CAT GGA GTG GC/3IABKFQ 3′. Final concentration of primer and probes was 900 nM and 250 nM, respectively. The following ddPCR program was optimized to amplify a 516 bp and 512 bp for the targeted and reference amplicon, respectively: 1-95° C.′ for 10 min, ramp 1 ºC/sec, 2-94ºC for 30 sec, ramp 1° C./sec, 3-61.7ºC for 30 sec, ramp 1° C./sec, 4-72ºC for 2 min, ramp 1° C./sec, 5-repeat steps 2-4 for 50 cycles, 6-98° C. for 10 min, ramp 1 ºC/sec, 7-4° C., ramp 1 ºC/sec. Bio-Rad Droplet Reader and QuantaSoft software were used generate and analyzed data, per manufacturer's guidelines. Absolute quantification (DNA/ul) was determined for reference and targeted genes. Total percent targeting was calculated as a ratio of HEX to FAM signal.

### Colony-Forming Units (CFU) Assay

Single live HSPCs were analyzed, as we previously described [16]. To genotype the colonies for wild type, mono- and bi-allelic integration, a three primer-based RAG2-specific genotyping PCR-based protocol was designed as follows: F-WT: 5 TCACCTGTTCAAAAGTCCCC 3″, R-integrated: 5′ TGGTTGTGCTTCACGTCC 3′, and R-WT 5′AGATGGTGTCATTTTTGGCAATAGAG 3′. The PCR reaction contained 0.5 uM of primers, 150-200 ng genomic DNA, and 1× Phusion Master Mix High Fidelity, per manufacturer's guidelines. The following PCR settings amplified the integrated band of 758 bp, a wild type band of 1246 bp: 1-98ºC:30 sec: 2-98° C.: 10 sec: 3-63ºC:30 sec: 4-72° C.:30 sec; 5-repeat septs 2-4 for 30 cycles, 6-72ºC:7 min. 7-4° C.

### Transplantation of Human Genome Modified CD34⁺ HSPCs and Engraftment Assessment

Human engraftment studies using fresh CB-HSPCs were injected intra-hepatic (IH) into 2 days old NSG pups. Engraftment studies using frozen healthy donor PB-HSPCs were injected intra-femoral (IF) into 6-8 weeks old NSG-SGM3 mice. Engraftment studies designed to rescue the disease phenotype were carried out using frozen PB-HSPCs derived from one RAG2-SCID patient. The patient-derived CD34⁺ HSPCs was given subcutaneous injections for granulocyte colony-stimulation factor (G-CSF) (filgranstim, Neupogen; Amgen. Thousand Oaks, CA) for 5 consecutive days at 10-16 meg/kg/day and one dose of Pleraxifor for mobilization and apheresis (National Institution of Allergy and Infectious Disease IRB-approved protocol). PB-HSPCs were selected from the leukapheresis using Miltenyi CliMACS. Human engraftment design and mouse handling were carried out as per the approved Stanford University Administrative Panel on Lab Animal Care (APLAC). Cells used for engraftment were exposed to 4-5 days of ex vivo culture. IF and IH transplantation studies were carried out as we previously described [16]. The antibody panel used to analyze IH engrafiments, as we previously described [16]. The following antibody panel was used to analyze IF bone marrow engraftments. CD3-PerCP-Cy5.5 (clone: HiT3A, BioLegend); CD19-FITC (clone: HIB19, BioLegend); mCD45.1-PE Cy7 (clone:A20, BioLegend); CD10-PE Texas Red (clone:Hi10a, BD Biosciences); HLA A-B-C-APC-Cy7 (clone: W6/32, BioLegend). CD33-AF-700 (clone:WM53, eBioSciences); CD34-APC (clone:8G12, BD Pharmigen); hCD45-BV786 (clone: HI3A, BDHorizon); IgM-BV711 (clone:MHM-88, BioLegend); CD20-BV510 (clone:2H7, BioLegend); CD56-PacBlue (clone:MEM-188, BioLegend); CD16-BUV395 (clone:3G8, BD Pharmigen); Live/dead (Invitrogen). The following antibody panel was used to analyze IF spleen engraftments: TCR a/b-PerCp-Cy5.5 (clone:iP26. BioLegend); TCR g/d-FITC (clone:cl.B1, BioLegend); CD45RA-PE Texas Red (clone: HI100); CD8-APC (clone:HiT8a, BioLegend); CD4 (clone:OKT4, BioLegend); CD3-BV421 (clone:UCHT1, BD BioSciences), mCD45.1, HLA A-B-C, CD33 and hCD45 same as above.

### Variable Immunoglobulin M (IgM) Heavy Chain (Vh) B-Cells Analysis

RNA was isolated using RNeasy Mini Kit (QIAGEN, Hilden, Germany) from CD19⁺ B-cells sorted from the bone marrow of mice engrafted with human gene-targeted HSPCs. Total RNA was reverse transcribed with random primers using Superscript IV (Invitrogen, Carlsbad, CA, USA). cDNA was amplified in a standard PCR for 40 cycles with primers specific for the seven variable heavy chains and with fluorescent reverse primer for the IgM isotype, as previously described [73]. IgVH family: VH1 5′-TGGAGCTGAGSAGSCTGAGATCYGA-3′; VH2 5′-AACCCACASAGACCCTCAC-3′; VH3 5′-TCCCTKARACTCTCCTGTRCAGC-3′; VH4 5′-CTACAACCCSTCCCTCAAGAGT-3′; VH5 5′-CAGCACCGCCTACCTGCAGTGGAGC-3′; VH6 5′-TCCGGGGACAGTGTCTCT-3′; VH7 5′-CAGCACRGCATAYCTGCAGATCAG-3′; (IgH Y chain 5′-6Fam-AAGTAGTCCTIGACCAGGCAGC-3)′; IgH μ chain 5′-6Fam-GGAGACGAGGGGGAAAAGG-3′.

### High Throughput Sequencing (HTS) of TCRα and TCRβ

Bone marrow and spleen samples were collected at end-point engraftment analysis, as we previously described [16]. Genomic DNA was extracted using QIAamp DNA Micro Kit, Qiagen (Germantown, MD, USA). The TCRα (TRA) and TCRb (TRB) rearranged genomic products were amplified by multiplex PCR (Adaptive Biotechnologies Seattle, WA). Samples were analyzed using Adaptive Biotechnologies' assay-based computational techniques to minimize PCR amplification bias. The frequency of a given TRA or TRB sequence is representative of the frequency of that clonotype in the original sample. The PCR products were sequenced using the Illumina HiSeq platform. Custom algorithms were used to filter the raw sequences for errors and align the sequences to reference genome sequences. Subsequently, the data were analyzed using the ImmunoSeq's online tools. The frequency of productive and nonproductive TRA or TRB rearrangements were analyzed within both unique and total TRA and TRB sequences obtained from T cells. The distribution of the frequency of individual clonotypes (including TRAV/TRBV to TRAJ/TRBJ pairing) was analyzed within unique sequences. Heat map representation of the frequencies of individual TRAV to TRAJ gene pairs and sequence overlap and treemaps representing CDR3 within the sample was produced using R software version 3.6.3 (2020-02-29). ImmunoSeq™ set of online tools was used to analyze the top 1000 most frequent clones, Shannon Entropy index of Diversity [H].

### Karyotype Analysis

RAG2-SCID patient-derived PB-HSPCs mock or RNP only treated and shipped to WiCell Cytogenetics (Madison, WI, USA). G-band karyotyping analysis was performed on 20 cells derived from each condition.

### FACS Analysis

All fluorescence activating cell sorting (FACS) analyses for human engraftment studies were done on FACS Aria II Sort Instrument part of the FACS Facility Core at Stanford University, Institute of Stem Cell Biology and Regenerative Medicine.

### Statistical Analysis

Statistical analysis was done with Prism 9 (GraphPad software).

### Ethics and Animal Approval Statement

All the work described in this study was carried out in compliance with all relevant ethical regulations. The animal studies were reviewed, approved and monitored by the Stanford University IACUC committee.

### REFERENCES

1. Alt. F. W., et al., Function and control of recombination-activating gene activity. Ann N Y Acad Sci, 1992. 651: p. 277-94.
2. Wada, T., et al., Characterization of immune function and analysis of RAG gene mutations in Omenn syndrome and related disorders. Clin Exp Immunol, 2000. 119 (1): p. 148-55.
3. Delmonte, O. M., A. Villa, and L. D. Notarangelo, Immune dysregulation in patients with RAG deficiency and other forms of combined immune deficiency. Blood, 2020. 135 (9): p. 610-619.
4. Hassan, A., et al., Outcome of hematopoietic stem cell transplantation for adenosine deaminase-deficient severe combined immunodeficiency. Blood, 2012. 120 (17): p. 3615-24; quiz 3626.
5. van Til, N. P., et al., Recombination-activating gene 1 (Rag1)-deficient mice with severe combined immunodeficiency treated with lentiviral gene therapy demonstrate autoimmune Omenn-like syndrome. J Allergy Clin Immunol, 2014. 133 (4): p. 1116-23.
6. Capo, V., et al., Efficacy of lentivirus-mediated gene therapy in an Omenn syndrome recombination-activating gene 2 mouse model is not hindered by inflammation and immune dysregulation. J Allergy Clin Immunol, 2018. 142 (3): p. 928-941 e8.
7. Pike-Overzet. K., et al., Correction of murine Rag1 deficiency by self-inactivating lentiviral vector-mediated gene transfer. Leukemia, 2011. 25 (9): p. 1471-83.
8. Lagresle-Peyrou, C., et al., Restoration of human B-cell differentiation into NOD-SCID mice engrafted with gene-corrected CD34+ cells isolated from Artemis or RAG1-deficient patients. Mol Ther, 2008. 16 (2): p. 396-403.
9. Mostoslavsky, G., et al., Complete correction of murine Artemis immunodeficiency by lentiviral vector-mediated gene transfer. Proc Natl Acad Sci USA, 2006. 103 (44): p. 16406-11.
10. Yates, F., et al., Gene therapy of RAG-2−/− mice: sustained correction of the immunodeficiency. Blood, 2002. 100 (12): p. 3942-9.
11. Lagresle-Peyrou, C., et al., Long-term immune reconstitution in RAG-1-deficient mice treated by retroviral gene therapy: a balance between efficiency and toxicity. Blood, 2006. 107 (1): p. 63-72.
12. Dever, D. P., et al., CRISPR/Cas9 beta-globin gene targeting in human haematopoietic stem cells. Nature, 2016. 539 (7629): p. 384-389.
13. Genovese, P., et al., Targeted genome editing in human repopulating haematopoictic stem cells. Nature, 2014. 510 (7504): p. 235-240.
14. Rai, R., et al., Targeted gene correction of human hematopoietic stem cells for the treatment of Wiskott-Aldrich Syndrome. Nat Commun, 2020. 11 (1): p. 4034.
15. Hubbard, N., et al., Targeted gene editing restores regulated CD40L function in X-linked hyper-IgM syndrome. Blood, 2016. 127 (21): p. 2513-22.
16. Pavel-Dinu, M., et al., Gene correction for SCID-X1 in long-term hematopoietic stem cells. Nat Commun, 2019. 10 (1): p. 1634.
17. Seet, C. S., et al., Generation of mature T cells from human hematopoietic stem and progenitor cells in artificial thymic organoids. Nat Methods, 2017. 14 (5): p. 521-530.
18. Bosticardo, M., et al., Artificial thymic organoids represent a reliable tool to study T-cell differentiation in patients with severe T-cell lymphopenia. Blood Adv, 2020. 4 (12): p. 2611-2616.
19. Jung, D., et al., Mechanism and control of V(D)J recombination at the immunoglobulin heavy chain locus. Annu Rev Immunol, 2006. 24: p. 541-70.
20. Krangel, M. S., Mechanics of T cell receptor gene rearrangement. Curr Opin Immunol. 2009. 21 (2): p. 133-9.
21. Lee, Y. N., et al., Characterization of T and B cell repertoire diversity in patients with RAG deficiency. Sci Immunol, 2016. 1 (6).
22. Dvorak, C. C., et al., The genetic landscape of severe combined immunodeficiency in the United States and Canada in the current era (2010-2018). J Allergy Clin Immunol, 2019. 143 (1): p. 405-407.
23. Farmer, J. R., et al., Outcomes and Treatment Strategies for Autoimmunity and Hyperinflammation in Patients with RAG Deficiency. J Allergy Clin Immunol Pract, 2019. 7 (6): p. 1970-1985 e4.
24. Schuetz, C., et al., SCID patients with ARTEMIS vs RAG deficiencies following HCT: increased risk of late toxicity in ARTEMIS-deficient SCID. Blood, 2014. 123 (2): p. 281-9.
25. Haddad. E., et al., SCID genotype and 6-month posttransplant CD4 count predict survival and immune recovery. Blood, 2018. 132 (17): p. 1737-1749.
26. Mamcarz, E., et al., Lentiviral Gene Therapy Combined with Low-Dose Busulfan in Infants with SCID-X1. N Engl J Med, 2019. 380 (16): p. 1525−i534.
27. van Til, N. P., et al., Correction of murine Rag2 severe combined immunodeficiency by lentiviral gene therapy using a codon-optimized RAG2 therapeutic transgene. Mol Ther, 2012. 20 (10): p. 1968-80.
28. De Ravin, S. S., et al., CRISPR-Cas9 gene repair of hematopoietic stem cells from patients with X-linked chronic granulomatous discase. Sci Transl Med, 2017. 9 (372).
29. Bak, R. O., D. P. Dever, and M. H. Porteus, CRISPR/Cas9 genome editing in human hematopoietic stem cells. Nat Protoc, 2018. 13 (2): p. 358-376.
30. Charlesworth, C. T., et al., Identification of preexisting adaptive immunity to Cas9 proteins in humans. Nat Med, 2019. 25 (2): p. 249-254.
31. Vakulskas, C. A. and M. A. Behlke, Evaluation and Reduction of CRISPR Off-Target Cleavage Events. Nucleic Acid Ther, 2019. 29 (4): p. 167-174.
32. Sweeney, C. L., et al., Targeted Repair of RAG2 in X-CGD iPSCs Requires Retention of Intronic Sequences for Expression and Functional Correction. Mol Ther, 2017. 25 (2): p. 321-330.
33. Bolger, A. M., M. Lohse, and B. Usadel, Trimmomatic: a flexible trimmer for Illumina sequence data. Bioinformatics, 2014. 30 (15): p. 2114-20.
34. Faust, G. G. and I. M. Hall, SAMBLASTER: fast duplicate marking and structural variant read extraction. Bioinformatics, 2014. 30 (17): p. 2503-5.
35. Cibulskis, K., et al., Sensitive detection of somatic point mutations in impure and heterogeneous cancer samples. Nat Biotechnol, 2013. 31 (3): p. 213-9.
36. Montel-Hagen, A., et al., Organoid-Induced Differentiation of Conventional T Cells from Human Pluripotent Stem Cells. Cell Stem Cell, 2019. 24 (3): p. 376-389 e8.
37. Fischer A, Notarangelo L D, Neven B, Cavazzana M, Puck J M: Severe combined immunodeficiencies and related disorders. Nat Rev Dis Primers 2015, 1:15061.
38. Notarangelo L D, Kim M S, Walter J E, Lee Y N: Human RAG mutations: biochemistry and clinical implications. Nat Rev Immunol 2016, 16(4):234-246.
39. Liang H E, Hsu L Y, Cado D, Cowell L G, Kelsoe G, Schlissel M S: The “dispensable” portion of RAG2 is necessary for efficient V-to-DJ rearrangement during B and T cell development. Immunity 2002, 17(5):639-651.
40. Sobacchi C, Marrella V, Rucci F, Vezzoni P, Villa A: RAG-dependent primary immunodeficiencies. Hum Mutat 2006, 27(12): 1174-1184.
41. Delmonte O M, Schuetz C, Notarangelo L D: RAG Deficiency: Two Genes, Many Diseases. J Clin Immunol 2018, 38(6): 646-655.
42. Tirosh I, Yamazaki Y, Frugoni F, Ververs F A, Allenspach E J, Zhang Y, Burns S, Al-Herz W, Noroski L, Walter J E et al: Recombination activity of human recombination-activating gene 2 (RAG2) mutations and correlation with clinical phenotype. J Allergy Clin Immunol 2019, 143(2): 726-735.
43. Villa A, Santagata S, Bozzi F, Imberti L, Notarangelo L D: Omenn syndrome: a disorder of Rag1 and Rag2 genes. J Clin Immunol 1999, 19(2): 87-97.
44. Comeo B, Moshous D, Gungor T, Wulffraat N, Philippet P, Le Deist F L, Fischer A, de Villartay J P: Identical mutations in RAG1 or RAG2 genes leading to defective V(D)J recombinase activity can cause either T-B-severe combined immune deficiency or Omenn syndrome. Blood 2001, 97(9):2772-2776.
45. Ehl S, Schwarz K, Enders A, Duffner U, Pannicke U, Kuhr J, Mascart F, Schmitt-Graeff A, Niemeyer C, Fisch P: A variant of SCID with specific immune responses and predominance of gamma delta T cells. J Clin Invest 2005, 115(11):3140-3148.
46. Walter J E, Rosen L B, Csomos K, Rosenberg J M, Mathew D, Keszei M, Ujhazi B, Chen K, Lee Y N, Tirosh I et al: Broad-spectrum antibodies against self-antigens and cytokines in RAG deficiency. J Clin Invest 2015, 125(11):4135-4148.
47. De Ravin S S, Cowen E W, Zarember K A, Whiting-Theobald N L, Kuhns D B, Sandler N G, Douek D C, Pittaluga S, Poliani P L. Lee Y N et al: Hypomorphic Rag mutations can cause destructive midline granulomatous disease. Blood 2010, 116(8): 1263-1271.
48. Schuetz C, Huck K, Gudowius S, Megahed M, Feyen O, Hubner B, Schneider D T, Manfras B, Pannicke U, Willemze R et al: An immunodeficiency discase with RAG mutations and granulomas. N Engl J Med 2008, 358(19): 2030-2038.
49. Buchbinder D. Baker R, Lee Y N, Ravell J. Zhang Y. McElwee J, Nugent D, Coonrod E M, Durtschi J D, Augustine N H et al: Identification of patients with RAG mutations previously diagnosed with common variable immunodeficiency disorders. J Clin Immunol 2015, 35(2): 119-124.
50. Villa A, Notarangelo L D: RAG gene defects at the verge of immunodeficiency and immune dysregulation. Immunol Rev 2019. 287(1): 73-90.
51. Pai S Y, Logan B R, Griffith L M, Buckley R H, Parrott R E, Dvorak C C, Kapoor N, Hanson I C. Filipovich A H, Jyonouchi S et al: Transplantation outcomes for severe combined immunodeficiency, 2000-2009. N Engl J Med 2014. 371(5): 434-446.
52. Jinek M, Chylinski K, Fonfara I, Hauer M, Doudna J A, Charpentier E: A programmable dual-RNA-guided DNA endonuclease in adaptive bacterial immunity. Science 2012. 337(6096):816-821.
53. Hendel A, Bak R O, Clark J T, Kennedy A B, Ryan D E, Roy S. Steinfeld I, Lunstad B D. Kaiser R J, Wilkens A B et al: Chemically modified guide RNAs enhance CRISPR-Cas genome editing in human primary cells. Nat Biotechnol 2015, 33(9): 985-989.
54. Vakulskas C A, Dever D P, Rettig G R, Turk R. Jacobi A M, Collingwood M A, Bode N M, McNeill M S, Yan S, Camarena J et al: A high-fidelity Cas9 mutant delivered as a ribonucleoprotein complex enables efficient gene editing in human hematopoietic stem and progenitor cells. Nat Med 2018, 24(8): 1216-1224.
55. Porteus M H: A New Class of Medicines through DNA Editing. N Engl J Med 2019, 380(10): 947-959.
56. Goodwin M, Lee E, Lakshmanan U, Shipp S, Froessl L, Barzaghi F, Passerini L, Narula M, Sheikali A, Lec C M et al: CRISPR-based gene editing enables FOXP3 gene repair in IPEX patient cells. Sci Adv 2020, 6(19): caaz0571.
57. Vavassori V, Mercuri E, Marcovecchio G E, Castiello M C, Schiroli G, Albano L, Margulies C. Buquicchio F, Fontana E, Beretta S et al: Modeling, optimization, and comparable efficacy of T cell and hematopoietic stem cell gene editing for treating hyper-IgM syndrome. EMBO Mol Med 2021, 13(3):e13545.
58. Sweeney C L, Pavel-Dinu M, Choi U, Brault J, Liu T. Koontz S. Li L. Theobald N. Lee J, Bello E A et al: Correction of X-CGD patient HSPCs by targeted CYBB cDNA insertion using CRISPR/Cas9 with 53BP1 inhibition for enhanced homology-directed repair. Gene Ther 2021. 28(6): 373-390.
59. Schiroli G, Ferrari S, Conway A, Jacob A, Capo V, Albano L, Plati T, Castiello M C. Sanvito F. Gennery A R et al: Preclinical modeling highlights the therapeutic potential of hematopoietic stem cell gene editing for correction of SCID-X1. Sci Transl Med 2017, 9(411).
60. Cromer M K, Camarena J, Martin R M, Lesch B J, Vakulskas C A, Bode N M, Kurgan G, Collingwood M A, Rettig G R, Bchlke M A et al: Gene replacement of alpha-globin with beta-globin restores hemoglobin balance in beta-thalassemia-derived hematopoictic stem and progenitor cells. Nat Med 2021, 27(4):677-687.
61. Gardner C L, Pavel-Dinu M, Dobbs K, Bosticardo M, Reardon P K, Lack J, DeRavin S S, Le K, Bello E, Pala F et al: Gene Editing Rescues In vitro T Cell Development of RAG2-Deficient Induced Pluripotent Stem Cells in an Artificial Thymic Organoid System. J Clin Immunol 2021.
62. Cradick T J, Qiu P, Lee C M, Fine E J, Bao G: COSMID: A Web-based Tool for Identifying and Validating CRISPR/Cas Off-target Sites. Mol Ther Nucleic Acids 2014, 3:e214.
63. Berland A, Rosain J, Kaltenbach S, Allain V, Mahlaoui N, Melki I, Fievet A, Dubois d'Enghien C, Ouachce-Chardin M, Perrin L et al: PROMIDISalpha: A T-cell receptor alpha signature associated with immunodeficiencies caused by V(D)J recombination defects. J Allergy Clin Immunol 2019, 143(1): 325-334 e322.
64. Karo J M, Schatz D G, Sun J C: The RAG recombinase dictates functional heterogeneity and cellular fitness in natural killer cells. Cell 2014, 159(1):94-107.
65 Dobbs K, Tabellini G, Calzoni E, Patrizi O, Martinez P, Giliani S C, Moratto D, Al-Herz W, Cancrini C, Cowan M et al: Corrigendum: Natural Killer Cells from Patients with Recombinase-Activating Gene and Non-Homologous End Joining Gene Defects Comprise a Higher Frequency of CD56(bright) NKG2A(+++) Cells, and Yet Display Increased Degranulation and Higher Perforin Content. Front Immunol 2017, 8:1244.
66. Miyazaki K, Miyazaki M: The Interplay Between Chromatin Architecture and Lineage-Specific Transcription Factors and the Regulation of Rag Gene Expression. Front Immunol 2021, 12:659761.
67. Huggins M A, Jameson S C, Hamilton S E: Embracing microbial exposure in mouse research. J Leukoc Biol 2019, 105(1): 73-79.
68. Brugman M H, Wiekmeijer A S, van Eggermond M, Wolvers-Tettero I, Langerak A W, de Haas E F, Bystrykh L V, van Rood J J, de Haan G, Fibbe W E et al: Development of a diverse human T-cell repertoire despite stringent restriction of hematopoietic clonality in the thymus. Proc Natl Acad Sci USA 2015, 112(44): E6020-6027.
69. Borghesi L, Hsu L Y, Miller J P. Anderson M, Herzenberg L, Herzenberg L, Schlissel M S, Allman D, Gerstein R M: B lineage-specific regulation of V(D)J recombinase activity is established in common lymphoid progenitors. J Exp Med 2004, 199(4): 491-502.
70. Igarashi H, Gregory S C, Yokota T, Sakaguchi N, Kincade P W: Transcription from the RAG1 locus marks the earliest lymphocyte progenitors in bone marrow. Immunity 2002, 17(2): 117-130.
71. Dobbs K, Tabellini G, Calzoni E, Patrizi O, Martinez P, Giliani S C, Moratto D, Al-Herz W, Cancrini C, Cowan M et al: Natural Killer Cells from Patients with Recombinase-Activating Gene and Non-Homologous End Joining Gene Defects Comprise a Higher Frequency of CD56(bright) NKG2A(+++) Cells, and Yet Display Increased Degranulation and Higher Perforin Content. Front Immunol 2017, 8:798.
72. Denning S M, Tuck D T, Singer K H, Haynes B F: Human thymic epithelial cells function as accessory cells for autologous mature thymocyte activation. J Immunol 1987, 138(3):680-686.
73. Silva H M, Takenaka M C, Moraes-Vieira P M, Monteiro S M, Hernandez M O, Chaara W, Six A, Agena F, Sesterheim P, Barbe-Tuana F M et al: Preserving the B-cell compartment favors operational tolerance in human renal transplantation. Mol Med 2012. 18:733-743.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### Exemplary Embodiments

Exemplary embodiments provided in accordance with the presently disclosed subject matter include, but are not limited to, the claims and the following embodiments:

Embodiment 1: a method of genetically modifying a cell from a subject with a Recombination-Activating Gene 2 (RAG2) deficiency, the method comprising: introducing into a cell isolated from the subject a single guide RNA (sgRNA) targeting the RAG2 gene, an RNA-guided nuclease, and a homologous donor template comprising a RAG2 cDNA comprising a nucleotide sequence having at least 80% identity to SEQ ID NO:5 or SEQ ID NO:6, flanked by a first and a second RAG2 homology region; wherein: the sgRNA binds to the nuclease and directs it to a target sequence within the RAG2 gene, whereupon the nuclease cleaves the gene at the target sequence, and wherein: the cDNA is integrated by homology directed recombination (HDR) at the site of the cleaved RAG2 gene, such that the cDNA is expressed under the control of the endogenous RAG2 promoter, thereby providing functional RAG2 protein product in the cell.
Embodiment 2: the method of embodiment 1, wherein the method further comprises isolating the cell from the subject prior to the introducing of the sgRNA, RNA-guided nuclease, and homologous donor template.
Embodiment 3: the method of embodiment 1 or 2, wherein the cell is an induced pluripotent stem cell (iPSC).
Embodiment 4: the method of embodiment 3, wherein the iPSC is derived from a fibroblast isolated from the subject.
Embodiment 5: the method of embodiment 1 or 2, wherein the cell is a hematopoietic stem and progenitor cell (HSPC).
Embodiment 6: the method of any one of embodiments 1 to 5, wherein the target sequence of the sgRNA is within exon 3 of the RAG2 gene, and wherein the RAG2 cDNA comprises exon 3 of the RAG2 gene.
Embodiment 7: the method of any one of embodiments 1 to 6, wherein the sgRNA comprises a nucleotide sequence complementary to a sequence selected from the group consisting of SEQ ID NO:1, SEQ ID NO:2, SEQ ID NO:3, or SEQ ID NO:4.
Embodiment 8: the method of embodiment 7, wherein the sgRNA comprises a nucleotide sequence complementary to SEQ ID NO: 1.
Embodiment 9: the method of any one of embodiments 1 to 8, wherein the sgRNA comprises 2′-O-methyl-3′-phosphorothioate (MS) modifications at one or more nucleotides.
Embodiment 10: the method of embodiment 9, wherein the 2′-O-methyl-3′-phosphorothioate (MS) modifications are present at the three terminal nucleotides of the 5′ and 3′ ends.
Embodiment 11: the method of any one of embodiments 1 to 10, wherein the RNA-guided nuclease is Cas9.
Embodiment 12: the method of embodiment 11, wherein the Cas9 is a high fidelity Streptococcus pyogenes Cas9 (SpCas9).
Embodiment 13: the method of any one of embodiments 1 to 12, wherein the sgRNA and the RNA-guided nuclease are introduced into the cell as a ribonucleoprotein (RNP).
Embodiment 14: the method of embodiment 13, wherein the RNP is introduced into the cell by electroporation.
Embodiment 15: the method of any one of embodiments 1 to 14, wherein the RAG2 cDNA comprises a nucleotide sequence having at least 85%, 90%, 95%, 96%, 97%, 98%, 99% or more identity to SEQ ID NO:5 or SEQ ID NO:6.
Embodiment 16: the method of embodiment 15, wherein the RAG2 cDNA comprises the nucleotide sequence of SEQ ID NO:5 or SEQ ID NO:6.
Embodiment 17: the method of any one of embodiments 1 to 16, wherein the homologous donor template further comprises a polyadenylation signal at the 3′ end of the cDNA, wherein both the cDNA and the polyadenylation signal are flanked by the first and the second RAG2 homology regions on the template.
Embodiment 18: the method of embodiment 17, wherein the polyadenylation signal is a bovine growth hormone polyadenylation signal.
Embodiment 19: the method of any one of embodiments 1 to 18, wherein the first and/or second RAG2 homology region comprises nucleotides 1-447 or 2848-3247 of SEQ ID NO:7, or a contiguous portion of nucleotides 1-447 or 2848-3247 of SEQ ID NO:7.
Embodiment 20: the method of embodiment 19, wherein the first and second RAG2 homology regions comprise nucleotides 1-447 or 2848-3247 of SEQ ID NO:7.
Embodiment 21: the method of any one of embodiments 1 to 20, wherein the homologous template further comprises a woodchuck hepatitis virus post-transcriptional regulatory element (WPRE).
Embodiment 22: the method of any one of embodiments 1 to 21, wherein the homologous donor template is introduced into the cells using a recombinant adeno-associated virus (rAAV) serotype 6 vector.
Embodiment 23: the method of any one of embodiments 1 to 22, wherein the genetically modified cell is heterozygous for the integrated RAG2 cDNA.
Embodiment 24: the method of any one of embodiments 1 to 22, wherein the genetically modified cell is homozygous for the integrated RAG2 cDNA.
Embodiment 25: the method of any one of embodiments 1 to 24, wherein the genetically modified cell can differentiate into a human embryonic mesodermal progenitor (hEMP) cell in vitro.
Embodiment 26: the method of any one of embodiments 1 to 25, wherein the genetically modified cell can differentiate into a T cell in vitro.
Embodiment 27: the method of embodiment 26, wherein the T cell is selected from the group consisting of CD34⁺ CD7⁺ CD5⁻; CD7⁺, CD5⁺, CD1a⁺; CD4⁺ CD8⁺; and CD3⁺ TCRαβ+; CD3⁺ TCRγδ⁺.
Embodiment 28: the method of any one of embodiments 1 to 27, wherein the RAG2 deficiency is a severe combined immunodeficiency (SCID).
Embodiment 29: the method of embodiment 28, wherein the SCID is a typical SCID.
Embodiment 30: the method of embodiment 28, wherein the SCID is an atypical SCID.
Embodiment 31: a method of treating a subject with a RAG2 deficiency, comprising (i) genetically modifying a cell from the subject using the method of any one of embodiments 1 to 30, and (ii) reintroducing the cell into the subject.
Embodiment 32: the method of embodiment 31, wherein the cell is reintroduced into the subject by systemic transplantation.
Embodiment 33: the method of embodiment 32, wherein the systemic transplantation comprises intravenous administration.
Embodiment 34: the method of embodiment 31, wherein the cell is reintroduced into the subject by local transplantation.
Embodiment 35: the method of embodiment 34, wherein the local transplantation comprises interfemoral administration.
Embodiment 36: the method of any one of embodiments 31 to 35, wherein the cell is cultured and/or selected prior to being reintroduced into the subject.
Embodiment 37: the method of any one of embodiments 31 to 36, wherein the cell is an iPSC.
Embodiment 38: the method of any one of embodiments 31 to 36, wherein the cell is an HSPC.
Embodiment 39: an sgRNA that specifically targets exon 3 of the RAG2 gene, wherein the sgRNA comprises a nucleotide sequence complementary to the sequence of any one of SEQ ID NOS: 1-4.
Embodiment 40: the sgRNA of embodiment 39, wherein the sgRNA comprises a nucleotide sequence complementary to the sequence of SEQ ID NO:1.
Embodiment 41: the sgRNA of embodiment 39 or 40, wherein the sgRNA comprises 2′-O-methyl-3′-phosphorothioate (MS) modifications at one or more nucleotides.
Embodiment 42: the sgRNA of embodiment 41, wherein the 2′-O-methyl-3′-phosphorothioate (MS) modifications are present at the three terminal nucleotides of the S′ and 3′ ends.
Embodiment 43: a homologous donor template comprising: (i) a RAG2 cDNA comprising a nucleotide sequence comprising at least 80% identity to SEQ ID NO:5 or SEQ ID NO:6; (ii) a first RAG2 homology region located to one side of the cDNA within the donor template; and (iii) a second RAG2 homology region located to the other side of the cDNA within the donor template.
Embodiment 44: the homologous donor template of embodiment 43, wherein the first RAG2 homology region comprises nucleotides 1-447 of SEQ ID NO:7, or a contiguous portion thereof, and the second RAG2 homology region comprises nucleotides 2848-3247 of SEQ ID NO:7, or a contiguous portion thereof.
Embodiment 45: the homologous donor template of embodiment 43 or 44, wherein the RAG2 cDNA comprises exon 3 of the RAG2 gene.
Embodiment 46: the homologous donor template of any one of embodiments 43 to 45, wherein the RAG2 cDNA comprises a nucleotide sequence having at least 85%, 90%, 95%, 96%, 97%, 98%, 99% or more identity to SEQ ID NO:5 or 6.
Embodiment 47: the homologous donor template of embodiment 46, wherein the RAG2 cDNA comprises the nucleotide sequence of SEQ ID NO:5 or 6.
Embodiment 48: the homologous donor template of any one of embodiments 43 to 47, wherein the RAG2 cDNA is codon optimized.
Embodiment 49: the homologous donor template of any one of embodiments 43 to 48, further comprising a polyadenylation signal at the 3′ end of the RAG2 cDNA, wherein both the cDNA and the polyadenylation signal are flanked by the first and second RAG2 homology regions on the template.
Embodiment 50: the donor template of embodiment 49, wherein the polyadenylation signal is a bovine growth hormone polyadenylation signal.
Embodiment 51: the donor template of embodiment 50, wherein the template comprises the sequence of SEQ ID NO: 7.
Embodiment 52: the donor template of any one of embodiments 43 to 51, further comprising a selectable marker.
Embodiment 53: the donor template of any one of embodiments 43 to 52, further comprising a woodchuck hepatitis virus post-transcriptional regulatory element (WPRE).
Embodiment 54: an isolated iPSC or HSPC comprising the sgRNA of any one of embodiments 39 to 42, or a homologous donor template of any one of embodiments 43 to 53.
Embodiment 55: an isolated, genetically modified iPSC or HSPC comprising an exogenous, codon-optimized RAG2 cDNA integrated at the endogenous RAG2 locus, wherein the integrated cDNA comprises a nucleotide sequence having at least 80% identity to SEQ ID NO:5 or 6.
Embodiment 56: the iPSC or HSPC of embodiment 55, wherein the RAG2 cDNA comprises a nucleotide sequence having at least 85%, 90%, 95%, 96%, 97%, 98%, 99% or more identity to SEQ ID NO:5 or 6.
Embodiment 57: the iPSC or HSPC of embodiment 56, wherein the RAG2 cDNA comprises the nucleotide sequence of SEQ ID NO:5 or 6.
Embodiment 58: the iPSC or HSPC of any one of embodiments 55 to 57, wherein the exogenous RAG2 cDNA comprises exon 3 of the RAG2 gene, and wherein the cDNA is integrated at exon 3 of the endogenous RAG2 gene.
Embodiment 59: the genetically modified iPSC or HSPC of any one of embodiments 55 to 58, wherein the HSPC was modified using the method of any one of embodiments 1 to 30.
Embodiment 60: a pharmaceutical composition comprising a plurality of genetically modified iPSCs or HSPCs comprising an exogenous, codon-optimized RAG2 cDNA integrated at the endogenous RAG2 locus, wherein the integrated cDNA comprises a nucleotide sequence having at least 80% identity to SEQ ID NO:5 or 6.
Embodiment 61: the pharmaceutical composition of embodiment 60, wherein the composition further comprises non-genetically modified iPSCs or HSPCs and/or iPSCs or HSPCs comprising INDELs at the RAG2 locus.
Embodiment 62: the pharmaceutical composition of embodiment 61, wherein the composition is comprised of at least 5% of genetically modified iPSCs or HSPCs comprising the integrated RAG2 cDNA.
Embodiment 63: the pharmaceutical composition of embodiment 62, wherein the composition is comprised of 9% to 50% of genetically modified iPSCs or HSPCs comprising the integrated RAG2 cDNA.

INFORMAL SEQUENCE LISTING
sgRNA-3
SEQ ID NO: 1
5′-TGCAGAGACATAGTTTCTGA-3′
sgRNA-5
SEQ ID NO: 2
5′-AGAAACTATGTCTCTGCAGA-3′
sgRNA-6
SEQ ID NO: 3
5′-AACATAGCCTTAATTCAGCC-3′
sgRNA-7
SEQ ID NO: 4
5′-TGAGAAGCCTGGCTGAATTA-3′
Codon optimized RAG2 cDNA version 1
SEQ ID NO: 5
ATGTCACTCCAAATGGTGACCGTGAGCAACAATATTGCTCTCATC
CAACCGGGTTTCTCTCTCATGAACTTCGACGGCCAGGTGTTTTTC
TTCGGGCAGAAGGGCTGGCCAAAACGGTCATGCCCGACTGGTGTA
TTTCATTTGGACGTGAAGCACAATCATGTGAAGCTCAAGCCGACT
ATCTTCAGCAAGGACAGCTGTTACCTGCCCCCGCTTAGGTATCCT
GCAACATGTACATTTAAGGGCTCCCTGGAAAGCGAGAAGCACCAG
TATATTATCCACGGCGGCAAGACCCCTAATAACGAAGTTAGCGAC
AAGATATACGTGATGAGTATAGTGTGCAAAAACAATAAGAAGGTC
ACGTTTCGATGCACCGAGAAGGACCTCGTGGGAGACGTGCCGGAG
GCTCGCTACGGCCATAGCATCAATGTTGTATATAGTAGAGGAAAG
TCTATGGGCGTACTTTTCGGCGGCCGATCTTATATGCCCTCAACA
CATCGCACGACTGAGAAGTGGAACTCAGTGGCAGATTGCCTCCCT
TGCGTGTTCCTGGTCGACTTCGAGTTCGGTTGCGCTACCTCATAT
ATACTTCCCGAGCTGCAGGACGGACTGTCTTTCCATGTGTCAATC
GCGAAGAACGATACCATCTACATCCTCGGTGGTCACAGCCTCGCG
AACAACATTCGACCTGCAAATCTGTACAGGATTAGAGTAGACCTC
CCGCTTGGGAGTCCCGCGGTCAATTGTACTGTCCTTCCGGGGGGC
ATATCCGTCAGCTCCGCCATCCTGACCCAGACCAATAACGACGAG
TTCGTCATTGTCGGTGGTTATCAACTTGAGAATCAGAAGAGGATG
ATATGTAACATCATCAGCCTTGAAGACAACAAAATAGAGATTAGA
GAGATGGAGACGCCCGACTGGACACCCGACATCAAACATAGCAAA
ATCTGGTTCGGCTCAAACATGGGCAACGGGACGGTGTTCCTGGGC
ATCCCTGGAGATAACAAGCAGGTGGTGTCCGAGGGGTTTTACTTC
TACATGTTGAAGTGCGCGGAGGACGACACCAACGAGGAACAGACT
ACATTTACCAACTCCCAAACAAGTACCGAGGACCCCGGCGATTCT
ACACCCTTCGAGGATTCAGAAGAGTTTTGCTTCAGCGCTGAAGCT
AACTCTTTCGACGGCGATGACGAGTTCGATACATACAACGAGGAC
GACGAGGAGGACGAAAGCGAAACTGGATACTGGATCACCTGCTGC
CCCACGTGCGACGTGGACATAAACACCTGGGTGCCGTTCTACTCA
ACCGAACTGAATAAGCCAGCAATGATCTACTGTTCACATGGTGAT
GGACACTGGGTGCACGCCCAGTGCATGGACCTCGCTGAGAGAACT
CTCATTCACTTGAGTGCAGGATCTAACAAATATTATTGCAACGAG
CACGTGGAGATCGCCCGCGCACTTCACACACCGCAGCGAGTGTTG
CCCTTGAAGAAGCCCCCTATGAAGAGCCTTAGGAAGAAGGGCAGC
GGCAAGATACTGACGCCCGCGAAAAAGAGCTTCCTGAGGAGACTG
TTCGACTAA
Codon optimized RAG2 cDNA version 2
SEQ ID NO: 6
ATGAGCCTGCAGATGGTGACCGTGTCCAACAATATCGCCCTGATC
CAGCCAGGCTTTTCTCTGATGAATTTCGACGGCCAGGTGTTCTTT
TTCGGCCAGAAGGGATGGCCAAAGCGGAGCTGCCCAACCGGCGTG
TTTCACCTGGACGTGAAGCACAACCACGTGAAGCTGAAGCCTACA
ATCTTCTCTAAGGATAGCTGCTACCTGCCCCCTCTGAGATACCCC
GCCACCTGTACATTCAAGGGCTCCCTGGAGTCTGAGAAGCACCAG
TACATCATCCACGGCGGCAAGACACCTAACAATGAGGTGTCTGAT
AAAATCTACGTGATGAGCATCGTGTGCAAGAACAATAAGAAGGTG
ACCTTTOGGTGTACAGAGAAGGACCTGGTGGGCGATGTGCCAGAG
GCAAGATACGGCCACAGCATCAATGTGGTGTATTCCAGAGGCAAG
TCTATGGGCGTGCTGTTCGGCGGCAGGAGCTACATGCCCTCCACC
CACCGCACCACAGAGAAGTGGAACTCCGTGGCCGACTGCCTGCCT
TGCGTGTTCCTGGTGGATTTTGAGTTCGGCTGTGCCACCTCTTAT
ATCCTGCCTGAGCTGCAGGACGGCCTGTCTTTCCACGTGAGCATC
GCCAAGAATGATACAATCTACATCCTGGGCGGCCACTCCCTGGCC
AACAATATCAGGCCAGCCAATCTGTATCGGATCAGAGTGGACCTG
CCTCTGGGCAGCCCAGCAGTGAACTGCACCGTGCTGCCAGGAGGC
ATCAGCGTGAGCAGCGCCATCCTGACCCAGACAAACAATGACGAG
TTTGTGATCGTGGGCGGCTACCAGCTGGAGAACCAGAAGAGGATG
ATCTGTAATATCATCAGCCTGGAGGATAACAAGATCGAGATCCGC
GAGATGGAGACCCCCGACTGGACACCTGACATCAAGCACAGCAAG
ATTTGGTTTGGCTCCAACATGGGCAATGGCACAGTGTTCCTGGGC
ATCCCCGGCGATAACAAGCAGGTGGTGTCCGAGGGCTTTTACTTC
TATATGCTGAAGTGCGCCGAGGACGATACCAATGAGGAGCAGACC
ACCTTCACCAACAGCCAGACCTCCACAGAGGACCCTGGCGATTCT
ACCCCATTCGAGGACAGCGAGGAGTTTTGTTTCTCCGCCGAGGCC
AATTCTTTTGACGGCGACGATGAGTTCGATACCTACAACGAGGAC
GATGAGGAGGATGAGAGCGAGACAGGCTATTGGATCACCTGCTGT
CCTACATGCGACGTGGACATCAATACCTGGGTGCCATTCTACTCC
ACAGAGCTGAACAAGCCCGCCATGATCTATTGCTCTCACGGCGAC
GGACACTGGGTGCACGCACAGTGTATGGATCTGGCCGAGAGGACC
CTGATCCACCTGTCTGCCGGCAGCAACAAGTACTATTGTAATGAG
CACGTGGAGATCGCCCGGGCCCTGCACACACCACAGAGAGTGCTG
CCTCTGAAGAAGCCACCCATGAAGTCCCTGCGCAAGAAGGGCTCT
GGCAAGATCCTGACCCCCGCCAAGAAGTCCTTTCTGCGGAGACTG
TTCGACTGA
RAG2 construct. Strategy is to knock in RAG2
cDNA into Exon 1 of RAG2 gene (i.e., exon
3 of overall gene, which includes two 5′ UTR
sequences separated by introns; exon 1 of
coding sequence), thereby restoring gene
expression.
Left homology arm: 1-447 bp
RAGI cDNA: 448-2031 bp
WPRE: 2032-2620 bp
BgH Poly A: 2621-2847 bp
Right homology arm: 2848-3247 bp
SEQ ID NO: 7
Tcctttgctcccgtctagtcacttcgcaccttcggctagtcttta
ttcacattccaggagtcccctctcccagagcaacaaagacagatt
tccaaagctacacatcccagagagaacggattcttgggaaatgtg
gttctttcagctgacgcatggtgactctttacggaaaaggactac
aattcccagaaatcctagggagcatatagtccgtggctaaaacat
gtcccagctccttggatggaatggcagtaaaggttctgtggctct
ttactgacctaactccttggattttcctcattcagtcccactgca
agcgtgtgggaggacttaaaaaaatgctattcacatgtgaaggaa
tctaaatacgatgattatatgaagcgatctctaagtcattttatt
ttataattctttcagACAAAAATCTACGTAGGATCAGAAACTATG
AGCCTGCAGATGGTGACCGTGTCCAACAATATCGCCCTGATCCAG
CCAGGCTTTTCTCTGATGAATTTCGACGGCCAGGTGTTCTTTTTC
GGCCAGAAGGGATGGCCAAAGCGGAGCTGCCCAACCGGCGTGTTT
CACCTGGACGTGAAGCACAACCACGTGAAGCTGAAGCCTACAATC
TTCTCTAAGGATAGCTGCTACCTGCCCCCTCTGAGATACCCCGCC
ACCTGTACATTCAAGGGCTCCCTGGAGTCTGAGAAGCACCAGTAC
ATCATCCACGGCGGCAAGACACCTAACAATGAGGTGTCTGATAAA
ATCTACGTGATGAGCATCGTGTGCAAGAACAATAAGAAGGTGACC
TTTCGGTGTACAGAGAAGGACCTGGTGGGCGATGTGCCAGAGGCA
AGATACGGCCACAGCATCAATGTGGTGTATTCCAGAGGCAAGTCT
ATGGGCGTGCTGTTCGGCGGCAGGAGCTACATGCCCTCCACCCAC
CGCACCACAGAGAAGTGGAACTCCGTGGCCGACTGCCTGCCTTGC
GTGTTCCTGGTGGATTTTGAGTTCGGCTGTGCCACCTCTTATATC
CTGCCTGAGCTGCAGGACGGCCTGTCTTTCCACGTGAGCATCGCC
AAGAATGATACAATCTACATCCTGGGCGGCCACTCCCTGGCCAAC
AATATCAGGCCAGCCAATCTGTATCGGATCAGAGTGGACCTGCCT
CTGGGCAGCCCAGCAGTGAACTGCACCGTGCTGCCAGGAGGCATC
AGCGTGAGCAGCGCCATCCTGACCCAGACAAACAATGACGAGTTT
GTGATCGTGGGCGGCTACCAGCTGGAGAACCAGAAGAGGATGATC
TGTAATATCATCAGCCTGGAGGATAACAAGATCGAGATCCGCGAG
ATGGAGACCCCCGACTGGACACCTGACATCAAGCACAGCAAGATT
TGGTTTGGCTCCAACATGGGCAATGGCACAGTGTTCCTGGGCATC
CCCGGCGATAACAAGCAGGTGGTGTCCGAGGGCTTTTACTTCTAT
ATGCTGAAGTGCGCCGAGGACGATACCAATGAGGAGCAGACCACC
TTCACCAACAGCCAGACCTCCACAGAGGACCCTGGCGATTCTACC
CCATTCGAGGACAGCGAGGAGTTTTGTTTCTCCGCCGAGGCCAAT
TCTTTTGACGGCGACGATGAGTTCGATACCTACAACGAGGACGAT
GAGGAGGATGAGAGCGAGACAGGCTATTGGATCACCTGCTGTCCT
ACATGCGACGTGGACATCAATACCTGGGTGCCATTCTACTCCACA
GAGCTGAACAAGCCCGCCATGATCTATTGCTCTCACGGCGACGGA
CACTGGGTGCACGCACAGTGTATGGATCTGGCCGAGAGGACCCTG
ATCCACCTGTCTGCCGGCAGCAACAAGTACTATTGTAATGAGCAC
GTGGAGATCGCCCGGGCCCTGCACACACCACAGAGAGTGCTGCCT
CTGAAGAAGCCACCCATGAAGTCCCTGCGCAAGAAGGGCTCTGGC
AAGATCCTGACCCCCGCCAAGAAGTCCTTTCTGCGGAGACTGTTC
GACTGAaatcaacctctggattacaaaatttgtgaaagattgact
ggtattcttaactatgttgctccttttacgctatgtggatacgct
gctttaatgcctttgtatcatgctattgcttcccgtatggctttc
attttctcctccttgtataaatcctggttgctgtctctttatgag
gagttgtggcccgttgtcaggcaacgtggcgtggtgtgcactgtg
tttgctgacgcaacccccactggttggggcattgccaccacctgt
cagctcctttccgggactttcgctttccccctccctattgccacg
gcggaactcatcgccgcctgccttgcccgctgctggacaggggct
cggctgttgggcactgacaattccgtggtgttgtcggggaagctg
acgtcctttccatggctgctcgcctgtgttgccacctggattctg
cgcgggacgtccttctgctacgtcccttcggccctcaatccagcg
gaccttccttcccgcggcctgctgccggctctgcggcctcttccg
cgtcttcgccttcgccctcagacgagtcggatctccctttgggcc
gcctccccgcagcctcgactgtgccttctagttgccagccatctg
ttgtttgcccctcccccgtgccttccttgaccctggaaggtgcca
ctcccactgtcctttcctaataaaatgaggaaattgcatcgcatt
gtctgagtaggtgtcattctattctggggggggggtggggcagga
cagcaagggggaggattgggaagacaatagcaggcatgctgggga
tgcggtgggctATGTCTCTGCAGATGGTAACAGTCAGTAATAACA
TAGCCTTAATTCAGCCAGGCTTCTCACTGATGAATTTTGATGGAC
AAGTTTTCTTCTTTGGACAAAAAGGCTGGCCCAAAAGATCCTGCC
CCACTGGAGTTTTCCATCTGGATGTAAAGCATAACCATGTCAAAC
TGAAGCCTACAATTTTCTCTAAGGATTCCTGCTACCTCCCTCCTC
TTCGCTACCCAGCCACTTGCACATTCAAAGGCAGCTTGGAGTCTG
AAAAGCATCAATACATCATCCATGGAGGGAAAACACCAAACAATG
AGGTTTCAGATAAGATTTATGTCATGTCTATTGTTTGCAAGAACA
ACAAAAAGGTTACTTTTCGCTGCACAGAGAAAGACTTGGTAGGAG
ATGTTC

1. A method of genetically modifying a cell from a subject with a Recombination-Activating Gene 2 (RAG2) deficiency, the method comprising:
introducing into a cell isolated from the subject a single guide RNA (sgRNA) targeting the RAG2 gene, an RNA-guided nuclease, and a homologous donor template comprising a RAG2 cDNA comprising a nucleotide sequence having at least 80% identity to SEQ ID NO:5 or SEQ ID NO:6, flanked by a first and a second RAG2 homology region; wherein:
the sgRNA binds to the nuclease and directs it to a target sequence within the RAG2 gene, whereupon the nuclease cleaves the gene at the target sequence, and wherein:
the cDNA is integrated by homology directed recombination (HDR) at the site of the cleaved RAG2 gene, such that the cDNA is expressed under the control of the endogenous RAG2 promoter, thereby providing functional RAG2 protein product in the cell.

2. (canceled)

3. The method of claim 1, wherein the cell is an induced pluripotent stem cell (iPSC).

4. (canceled)

5. The method of claim 1, wherein the cell is a hematopoietic stem and progenitor cell (HSPC).

6. The method of claim 1, wherein the target sequence of the sgRNA is within exon 3 of the RAG2 gene, and wherein the RAG2 cDNA comprises exon 3 of the RAG2 gene.

7. The method of claim 1, wherein the sgRNA comprises a nucleotide sequence complementary to a sequence selected from the group consisting of SEQ ID NO:1, SEQ ID NO:2, SEQ ID NO:3, or SEQ ID NO:4.

8-10. (canceled)

11. The method of claim 1, wherein the RNA-guided nuclease is Cas9.

12. (canceled)

13. The method of claim 1, wherein the sgRNA and the RNA-guided nuclease are introduced into the cell as a ribonucleoprotein (RNP).

14. (canceled)

15. The method of claim 1, wherein the RAG2 cDNA comprises a nucleotide sequence having at least 85%, 90%, 95%, 96%, 97%, 98%, 99% or more identity to SEQ ID NO:5 or SEQ ID NO:6.

16-21. (canceled)

22. The method of claim 1, wherein the homologous donor template is introduced into the cells using a recombinant adeno-associated virus (rAAV) serotype 6 vector.

23-27. (canceled)

28. The method of claim 1, wherein the RAG2 deficiency is a severe combined immunodeficiency (SCID).

29. (canceled)

30. (canceled)

31. A method of treating a subject with a RAG2 deficiency, comprising (i) genetically modifying a cell from the subject using the method of claim 1, and (ii) reintroducing the cell into the subject.

32-36. (canceled)

37. The method of claim 31, wherein the cell is an iPSC.

38. The method of claim 31, wherein the cell is an HSPC.

39-42. (canceled)

43. A homologous donor template comprising:
(i) a RAG2 cDNA comprising a nucleotide sequence comprising at least 80% identity to SEQ ID NO:5 or SEQ ID NO:6;
(ii) a first RAG2 homology region located to one side of the cDNA within the donor template; and
(iii) a second RAG2 homology region located to the other side of the cDNA within the donor template.

44. (canceled)

45. The homologous donor template of claim 43, wherein the RAG2 cDNA comprises exon 3 of the RAG2 gene.

46. The homologous donor template of claim 43, wherein the RAG2 cDNA comprises a nucleotide sequence having at least 85%, 90%, 95%, 96%, 97%, 98%, 99% or more identity to SEQ ID NO:5 or 6.

47-53. (canceled)

54. An isolated iPSC or HSPC comprising a homologous donor template of claim 43.

55. An isolated, genetically modified iPSC or HSPC comprising an exogenous, codon-optimized RAG2 cDNA integrated at the endogenous RAG2 locus, wherein the integrated cDNA comprises a nucleotide sequence having at least 80% identity to SEQ ID NO:5 or 6.

56. The iPSC or HSPC of claim 55, wherein the RAG2 cDNA comprises a nucleotide sequence having at least 85%, 90%, 95%, 96%, 97%, 98%, 99% or more identity to SEQ ID NO:5 or 6.

57. (canceled)

58. The iPSC or HSPC of claim 55, wherein the exogenous RAG2 cDNA comprises exon 3 of the RAG2 gene, and wherein the cDNA is integrated at exon 3 of the endogenous RAG2 gene.

59-63. (canceled)
